(19) United States
(12) Patent Application Publication
(10) Pub. No.: US 2023/0348456 A1
(43) Pub. Date: Nov. 2, 2023

(54) QUINAZOLINONES, PHARMACEUTICAL COMPOSITIONS CONTAINING THE SAME, AND METHODS OF USING THE SAME

(71) Applicant: Repare Therapeutics Inc., St-Laurent (CA)

(72) Inventors: Janek SZYCHOWSKI, Montreal (CA); Bingcan LIU, Montreal (CA); Evelyne DIETRICH, Laval (CA); Alexander PERRYMAN, Pointe-Claire (CA); Sheldon N. CRANE, Notre Dame de l'Ile Perrot (CA); Vouy Linh TRUONG, Pierrefonds (CA); Abbas ABDOLI, Montreal (CA); Alexanne BOUCHARD, Montreal (CA)

(73) Assignee: Repare Therapeutics Inc., St-Laurent (CA)

(21) Appl. No.: 18/140,302

(22) Filed: Apr. 27, 2023

Related U.S. Application Data
(60) Provisional application No. 63/336,827, filed on Apr. 29, 2022.

(51) Int. Cl. C07D 417/14; C07D 401/14; C07D 405/14; C07D 413/14; C07D 491/107; C07D 209/46; C07D 493/08; C07D 411/14

(52) U.S. Cl. CPC C07D 417/14; C07D 401/14; C07D 405/14; C07D 413/14; C07D 491/107; C07D 209/46; C07D 493/08; C07D 411/14

(57) ABSTRACT

Disclosed are compounds and pharmaceutically acceptable salts thereof that may be used in the treatment of subjects in need thereof. The compounds disclosed herein may be inhibitors of tyrosine and threonine-specific cdc2-inhibitory kinase (Myt1). Also disclosed are pharmaceutical compositions containing the compounds or pharmaceutically acceptable salts thereof and methods of their preparation and use.

### FIELD OF THE INVENTION

The invention relates to compounds and pharmaceutical compositions, their preparation and their use in the treatment of a disease or condition, e.g., cancer, and, in particular, those diseases or conditions (e.g., cancers that harbor CCNE1 amplification/overexpression or FBXW7-mutated cancers) which depend on the activity of membrane-associated tyrosine and threonine-specific cdc2-inhibitory kinase (Myt1).

### BACKGROUND

DNA is continuously subjected to both endogenous insults (e.g., stalled replication forks, reactive oxygen species) and exogenous insults (UV, ionizing radiation, chemical) that can lead to DNA damage. As a result, cells have established sophisticated mechanisms to counteract these deleterious events that would otherwise compromise genomic integrity and lead to genomic instability diseases such as cancer. These mechanisms are collectively referred to as the DNA damage response (DDR). One component of the overall DDR is the activation of various checkpoint pathways that modulate specific DNA-repair mechanisms throughout the various phases of the cell cycle, which includes the G1, S, G2 and Mitosis checkpoints. A majority of cancer cells have lost their G1 checkpoint owing to p53 mutations and as such, rely on the G2 checkpoint to make the necessary DNA damage corrections prior to committing to enter mitosis and divide into 2 daughter cells.

There is a need for new anti-cancer therapeutic approaches, e.g., those utilizing small-molecules, especially therapies allowing for targeted cancer treatment.

### SUMMARY OF THE INVENTION

In an aspect, the invention provides a compound of formula (I):

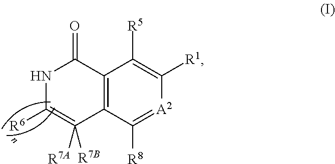

or a pharmaceutically acceptable salt thereof,
where
R¹ is:

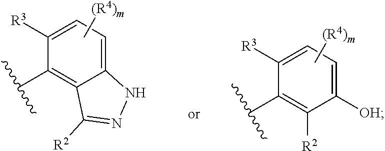

[symbol] is a single bond, and n is 0 or 1; or [symbol] is a double bond, and n is 1;
m is 0, 1, or 2;
A¹ is N, R^{7A} is absent, hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂, and R^{7B} is absent; or A¹ is C, and R^{7A} is hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂, and R^{7B} is absent, hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂;
A² is N or CH;
each of R² and R³ are independently hydrogen, halogen, optionally substituted C₁₋₆ alkyl, or optionally substituted C₃₋₈ cycloalkyl;
each R⁴ is independently halogen;
R⁵ is hydrogen, halogen, hydroxyl, optionally substituted C₁₋₆ alkyl, —N(R^{5A})₂, or —OR^{5B};
each R^{5A} is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl; or two R^{5A} groups, together with the atom to which both are attached, combine to form an optionally substituted C₂₋₉ heterocyclyl;
R^{5B} is optionally substituted C₁₋₆ alkyl;
R⁶ is hydrogen, halogen, optionally substituted C₁₋₆ alkyl, optionally substituted C₁₋₆ heteroalkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{6A}, —N(R^{6B})₂, or -L¹-R^{6C};
R^{6A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{6B} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{6C} is hydrogen, halogen, optionally substituted C₁₋₆ alkyl, optionally substituted C₂₋₆ alkenyl, optionally substituted C₂₋₆ alkynyl, optionally substituted acyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{6D}, or —N(R^{6E})₂;
R^{6D} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{6E} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{7C} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{7D} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R⁸ is hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{8A}, or -L²-R^{8B};
R^{8A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{8B} is hydrogen, halogen, cyano, optionally substituted C₁₋₆ alkyl, optionally substituted C₂₋₆ alkenyl, optionally substituted C₂₋₆ alkynyl, optionally substituted acyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{8C}, —N(R^{8D})₂, —CO₂H, —NHC(O)(R^{8E}), —C(O)N(R^{8E})₂;
R^{8C} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{8D} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{8E} is independently hydrogen, optionally substituted C₁₋₆ alkyl, optionally substituted C₁₋₆ heteroalkyl, optionally substituted C₃₋₈ cycloalkyl, or optionally substituted C₂₋₉ heterocyclyl; or two R^{8E} groups, together with the atom to which both are attached, combine to form an optionally substituted C₂₋₉ heterocyclyl; and
each of L¹ and L² is independently optionally substituted C₂₋₉ heterocyclylene, optionally substituted C₂₋₉ heteroarylene, optionally substituted C₆₋₁₀ arylene, or optionally substituted C₃₋₈ cycloalkylene.

In some embodiments, R⁶ is hydrogen. In some embodiments, R⁶ is optionally substituted C₆₋₁₀ aryl. In some embodiments, R⁶ is optionally substituted phenyl. In some embodiments, R⁶ is optionally substituted C₁₋₉ heteroaryl. In some embodiments, R⁶ is optionally substituted C₅ heteroaryl. In some embodiments, the optionally substituted C₅ heteroaryl contains one nitrogen atom.

In some embodiments, R⁶ is -L¹-R^{6C}. In some embodiments, R^{6C} is optionally substituted C₂₋₉ heterocyclyl. In some embodiments, R^{6C} is —OR^{6D}. In some embodiments, R^{6D} is optionally substituted C₁₋₆ alkyl. In some embodiments, R^{6C} is —N(R^{6E})₂. In some embodiments, each R^{6E} is independently hydrogen, optionally substituted C₁₋₆ alkyl, or optionally substituted C₂₋₉ heterocyclyl.

In some embodiments, L¹ is optionally substituted C₂₋₉ heteroarylene. In some embodiments, the optionally substituted C₂₋₉ heteroarylene is an optionally substituted C₅ heteroarylene. In some embodiments, the optionally substituted C₅ heteroarylene contains one nitrogen atom.

In some embodiments, R⁶ is optionally substituted C₁₋₆ alkyl. In some embodiments, R⁶ is optionally substituted C₂₋₉ heterocyclyl. In some embodiments, R⁶ is —OR^{6A}. In some embodiments, R^{6A} is phenyl. In some embodiments, R⁶ is optionally substituted C₁₋₆ heteroalkyl. In some embodiments, R⁶ is optionally substituted C₃₋₈ cycloalkyl. In some embodiments, R⁶ is optionally substituted cyclopropyl.

In some embodiments, R⁶ is:

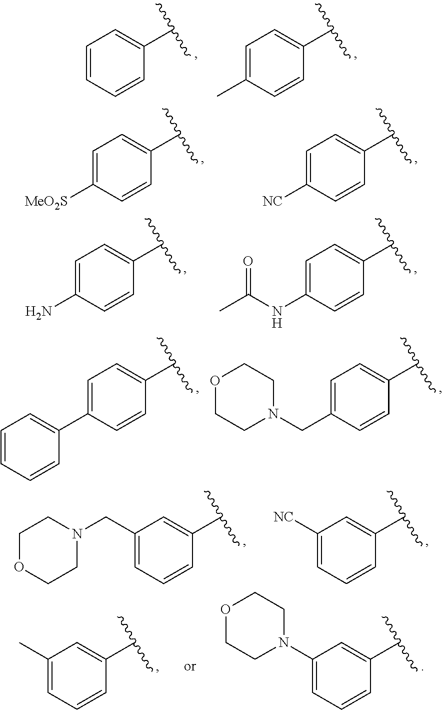

In some embodiments, R⁶ is:

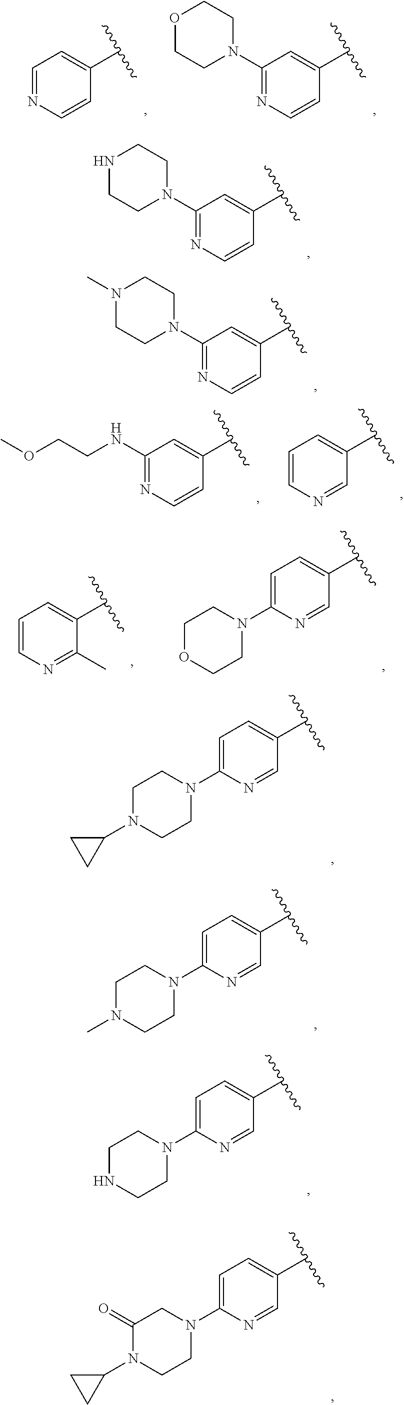

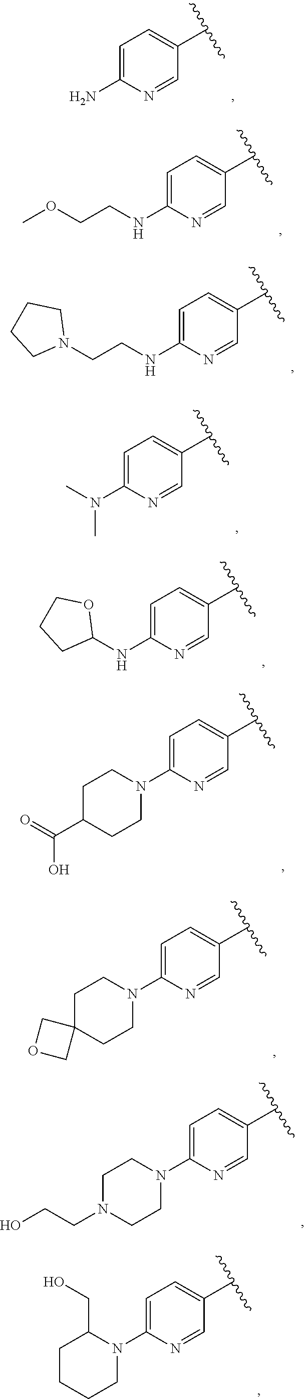

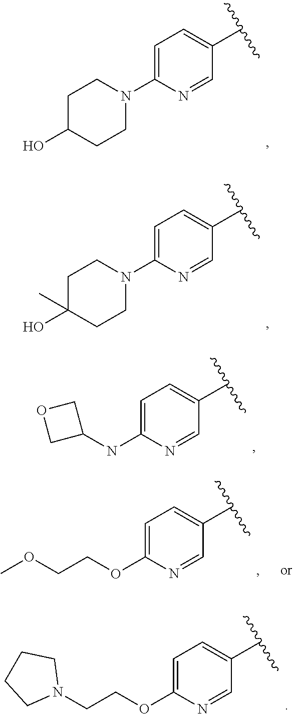 [SMILES: *c1ccc(N2CCC(O)CC2)nc1 ; *c1ccc(N2CCC(C)(O)CC2)nc1 ; *c1ccc(NC2COC2)nc1 ; *c1ccc(OCCOC)nc1 ; *c1ccc(OCCN2CCCC2)nc1]

In some embodiments, R⁶ is:

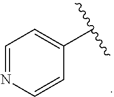 [SMILES: *c1ccncc1]

In some embodiments, R⁶ is:

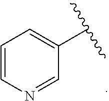 [SMILES: *c1cccnc1]

In some embodiments, R⁶ is:

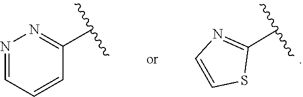 [SMILES: *c1cccnn1 ; *c1nccs1]

In some embodiments, R⁶ is:

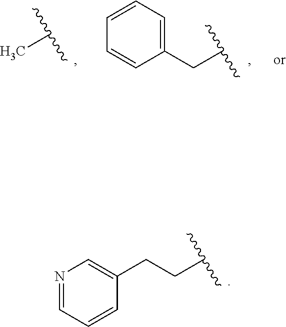

In some embodiments, R⁶ is:

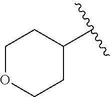 [SMILES: *C1CCOCC1]

In some embodiments, R⁶ is:

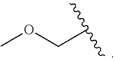 [SMILES: *COC]

In some embodiments, the compound is of formula (III-A):

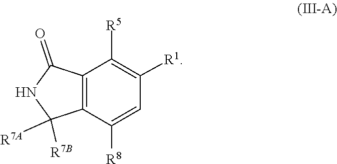

In some embodiments, the compound is of formula (III-B):

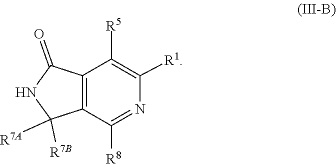

In some embodiments, R^{7B} is hydrogen.

In some embodiments, the compound is of formula (II-C):

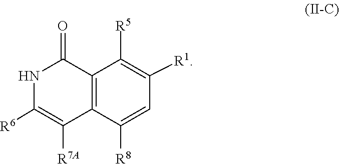

In some embodiments, R^{7A} is hydrogen.

In some embodiments, the compound is of formula (II-A):

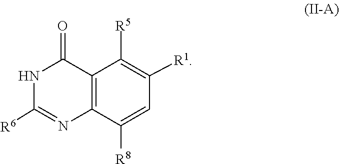

In some embodiments, the compound is of formula (II-B):

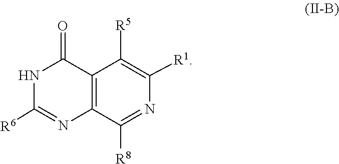

In some embodiments, R⁵ is N(R^{5A})₂. In some embodiments, R⁵ is NH₂.

In some embodiments, R¹ is:

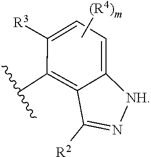

In some embodiments, R¹ is enriched for the atropisomer having the structure of:

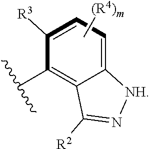

In some embodiments, R¹ is:

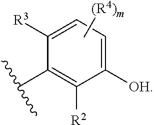

In some embodiments, R¹ is enriched for the atropisomer having the structure of:

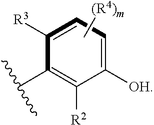

In some embodiments, m is 0. In some embodiments, m is 1. In some embodiments, m is 2.

In some embodiments, R⁴ is fluoro.

In some embodiments, R² is optionally substituted C₁₋₆ alkyl. In some embodiments, R² is hydrogen.

In some embodiments, R³ is optionally substituted C₁₋₆ alkyl. In some embodiments, R³ is hydrogen.

In some embodiments, R¹ is:

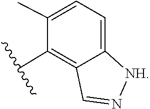 [SMILES: *c1c(C)ccc2[nH]ncc12]

In some embodiments, R¹ is:

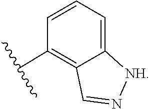 [SMILES: *c1cccc2[nH]ncc12]

In some embodiments, R¹ is:

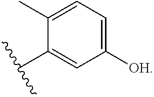 [SMILES: *c1cc(O)ccc1C]

In some embodiments, R¹ is:

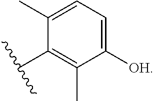 [SMILES: *c1c(C)ccc(O)c1C]

In some embodiments, R⁸ is hydrogen. In some embodiments, R⁸ is halogen. In some embodiments, R⁸ is —Br. In some embodiments, R⁸ is optionally substituted C₆₋₁₀ aryl. In some embodiments, R⁸ is optionally substituted phenyl. In some embodiments, R⁸ is optionally substituted C₂₋₉ heterocyclyl. In some embodiments, R⁸ is optionally substituted C₃₋₈ cycloalkyl. In some embodiments, R⁸ is optionally substituted C₁₋₉ heteroaryl. In some embodiments, R⁸ is optionally substituted C₁₋₉ heteroaryl containing exactly two nitrogens. In some embodiments, R⁸ is optionally substituted C₁₋₉ heteroaryl containing exactly one nitrogen. In some embodiments, R⁸ is -L²-R^{8B}.

In some embodiments, L² is optionally substituted C₆₋₁₀ arylene. In some embodiments, L² is optionally substituted phenylene. In some embodiments, L² is optionally substituted C₂₋₉ heteroarylene.

In some embodiments, -L²-R^{8B} is:

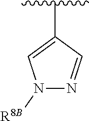

In some embodiments, -L²-R^{8B} is:

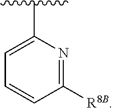

In some embodiments, -L²-R^{8B} is:

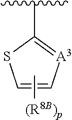

where
A³ is N or CH; and
p is 0, 1, or 2.

In some embodiments, p is 0. In some embodiments, p is 1. In some embodiments, p is 2.

In some embodiments, A³ is CH. In some embodiments, A³ is N.

In some embodiments, R^{8B} is cyano. In some embodiments, R^{8B} is halogen. In some embodiments, R^{8B} is Cl. In some embodiments, R^{8B} is optionally substituted acyl. In some embodiments, R^{8B} is —CO₂H. In some embodiments, In some embodiments, R^{8B} is —C(O)N(R^{8E})₂. In some embodiments, one R^{8E} is hydrogen. In some embodiments, one R^{8E} is optionally substituted C₁₋₆ heteroalkyl. In some embodiments, one R^{8E} is optionally substituted C₁₋₆ alkyl. In some embodiments, one R^{8E} is —CH₃ or —CH(CH₃)₂. In some embodiments, one R^{8E} is optionally substituted C₃₋₈ cycloalkyl. In some embodiments, one R^{8E} is optionally substituted cyclopropyl. In some embodiments, two R^{8E} groups, together with the atom to which both are attached, combine to form an optionally substituted C₂₋₉ heterocyclyl.

In some embodiments, —C(O)N(R^{8E})₂ is:

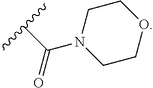 [SMILES: *C(=O)N1CCOCC1]

In some embodiments, R^{8B} is optionally substituted C₁₋₆ alkyl. In some embodiments, R^{8B} is —CF₃. In some embodiments, R^{8B} is OR^{8C}. In some embodiments, R^{8C} is optionally substituted C₁₋₆ alkyl or optionally substituted C₂₋₉ heterocyclyl.

In some embodiments, R⁸ is:

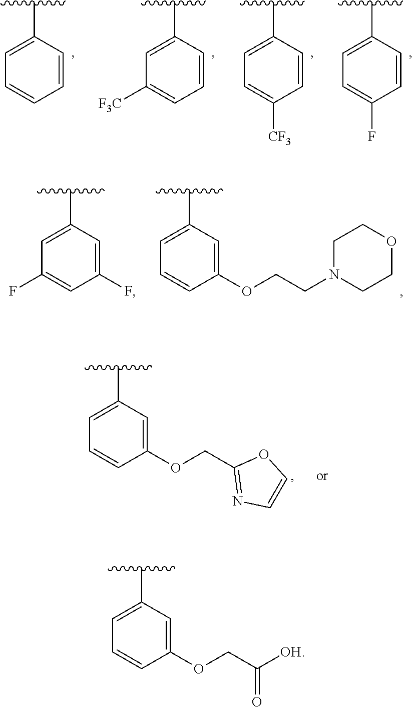

In some embodiments, R⁸ is:

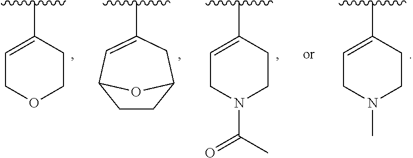

In some embodiments, R⁸ is:

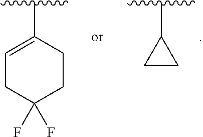

In some embodiments, R⁸ is:

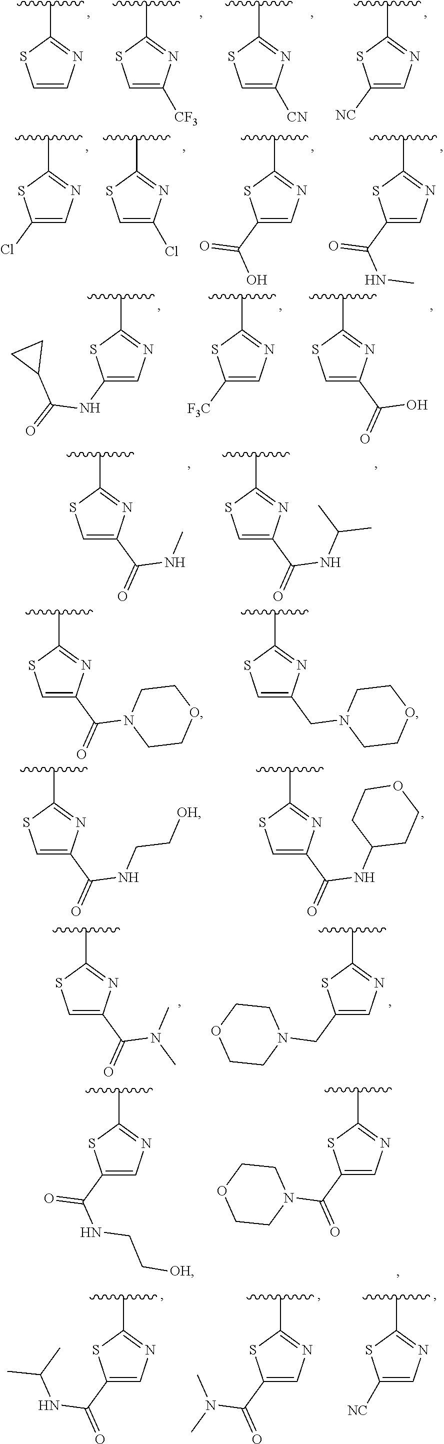

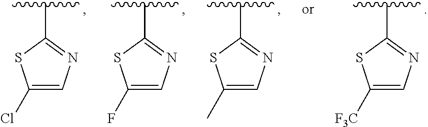

In some embodiments, R⁸ is:

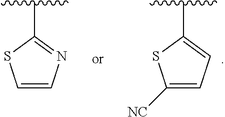 [SMILES: *c1nccs1 ; *c1ccc(C#N)s1]

In some embodiments, R⁸ is

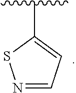 [SMILES: *c1ccns1]

In some embodiments, R⁸ is:

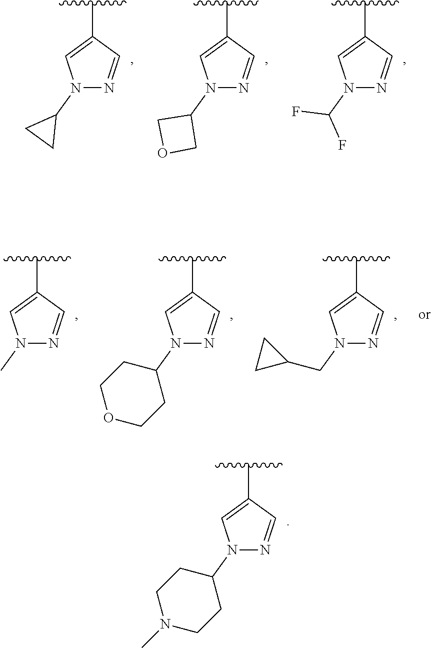

In some embodiments, R⁸ is:

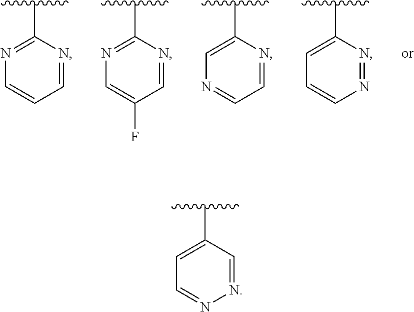

In some embodiments, R⁸ is:

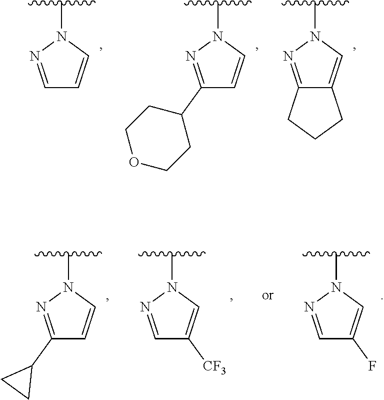

In some embodiments, R⁸ is:

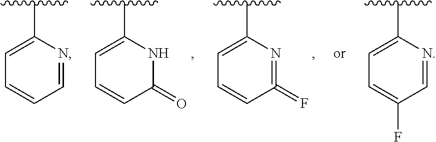

In some embodiments, R⁸ is:

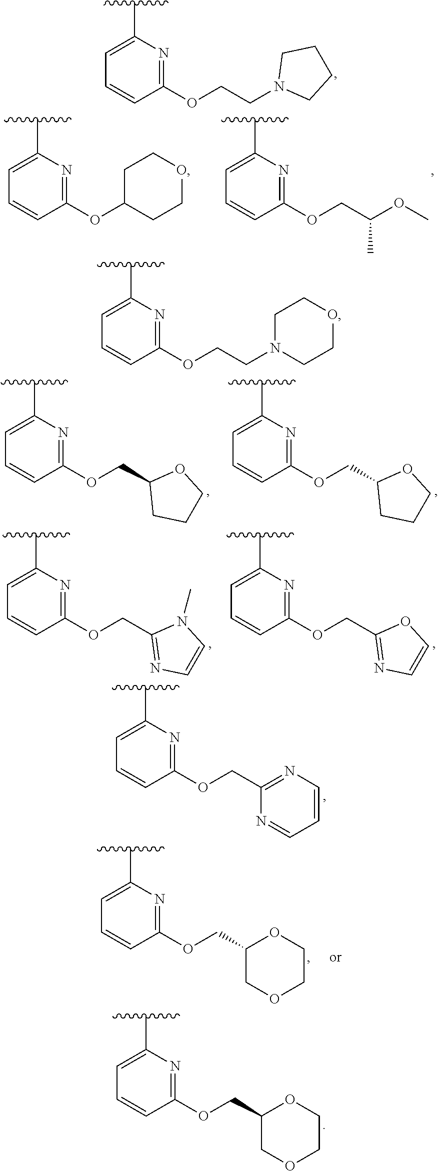

In some embodiments, the compound is selected from the group consisting of compounds 1 to 165 and pharmaceutically acceptable salts thereof.

In another aspect, the invention provides a pharmaceutical composition including the compound disclosed herein, or a pharmaceutically acceptable salt thereof, and a pharmaceutically acceptable excipient. In some embodiments, the composition is isotopically enriched in deuterium.

In yet another aspect, the invention provides a method of inhibiting Myt1 in a cell expressing Myt1, the method including contacting the cell with the compound disclosed herein.

In some embodiments, the cell is overexpressing CCNE1. In some embodiments, the cell is in a subject. In yet another aspect, the invention provides a method of inhibiting Myt1 in a cell expressing Myt1, the method including contacting the cell with the compound disclosed herein.

In some embodiments, the cell is overexpressing CCNE1. In some embodiments, the cell is in a subject.

In still another aspect, the invention provides a method of treating a subject in need thereof including administering to the subject the compound disclosed herein, or a pharmaceutically acceptable salt thereof, or the pharmaceutical composition disclosed herein.

In some embodiments, the subject is suffering from, and is in need of a treatment for, a disease or condition having the symptom of cell hyperproliferation. In some embodiments, the disease or condition is a cancer. In some embodiments, the cancer is a cancer overexpressing CCNE1.

In still another aspect, the invention provides a method of treating a cancer in a subject, the method including administering to the subject in need thereof a therapeutically effective amount of a Myt1 inhibitor, where the cancer has been previously identified as a cancer overexpressing CCNE1.

In another aspect, the invention provides a method of treating a cancer in a subject, the method including administering to the subject in need thereof a therapeutically effective amount of a Myt1 inhibitor, where the cancer is a cancer overexpressing CCNE1.

In yet another aspect, the invention provides a method of inducing cell death in a cancer cell overexpressing CCNE1, the method including contacting the cell with an effective amount of a Myt1 inhibitor.

In some embodiments, the cell is in a subject. In some embodiments, the Myt1 inhibitor is the compound disclosed herein or a pharmaceutically acceptable salt thereof. In some embodiments, the cancer overexpressing CCNE1 is uterine cancer, ovarian cancer, breast cancer, stomach cancer, esophageal cancer, lung cancer, or endometrial cancer.

In still another aspect, the invention provides a method of treating a cancer in a subject, the method including administering to the subject in need thereof a therapeutically effective amount of a Myt1 inhibitor, where the cancer has been previously identified as a cancer having an inactivating mutation in the FBXW7 gene.

In another aspect, the invention provides a method of treating a cancer in a subject, the method including administering to the subject in need thereof a therapeutically effective amount of a Myt1 inhibitor, where the cancer has an inactivating mutation in the FBXW7 gene.

In yet another aspect, the invention provides a method of inducing cell death in an FBXW7-mutated cancer cell, the method including contacting the cell with an effective amount of a Myt1 inhibitor.

In some embodiments, the cell is in a subject. In some embodiments, the cancer is uterine cancer, colorectal cancer, breast cancer, lung cancer, or esophageal cancer. In some embodiments, the Myt1 inhibitor is the compound disclosed herein, or a pharmaceutically acceptable salt thereof.

### ABBREVIATIONS

Abbreviations and terms that are commonly used in the fields of organic chemistry, medicinal chemistry, pharmacology, and medicine and are well known to practitioners in these fields are used herein. Representative abbreviations and definitions are provided below:
Ac is acetyl [CH₃C(O)—];
ACN is acetonitrile;
Ac₂O is acetic anhydride;
AcOH is acetic acid;
APC is antigen-presenting cell;
Ar is aryl;
aq. is aqueous;
9-BBN is 9-borabicyclo[3.3.1]nonane;
BINAP is (2,2′-bis(diphenylphosphino)-1,1′-binaphthyl);
Bn is benzyl;
Boc is tert Butyloxycarbonyl;
n-BuLi is n-butyl lithium;
CDI is carbonyldiimidazole;
cmpd is compound;
conc. is concentrated;
DCM is dichloromethane;
DIAD is diisopropylazodicarboxylate;
DIBAL is diisobutylaluminum hydride;
DIPEA is diisoproplyethyl amine;
DMA is dimethylacetamide;
DMAP is 4-dimethylaminopyridine;
DME is dimethoxyethane;
DMF is N,N-dimethylformamide;
DMSO is dimethyl sulfoxide;
dppf is 1,1′-bis(diphenylphosphino)ferrocene;
dtbpf is 1,1′-Bis(di-ted-butylphosphino)ferrocene;
EDAC (or EDC) is 1-ethyl-3-[3-(dimethylamino)propyl]-carbodiimide HCl;
ESI is electrospray ionization mass spectrometry;
Et₂O is diethyl ether;
Et₃N is triethylamine;
Et is ethyl;
EtOAc is ethyl acetate;
EtOH is ethanol;
3-F-Ph is 3-fluorophenyl,
h is hour;
HATU is (1-[bis(dimethylamino)methylene]-1H-1,2,3-triazolo[4,5-b]pyridinium 3-oxide
hexafluorophosphate;
HCl is hydrochloric acid;
Het is heteroaryl;
Hex is hexanes;
HOBt is 1-hydroxybenzotriazole;
HPLC is high performance liquid chromatography;
IPA is isopropanol;
IPAc is isopropyl acetate;
LCMS is HPLC with mass spectral detection;
LiHMDS is lithium bis(trimethylsilyl)amide;
LG is leaving group;
M is molar;
mCPBA is metachloroperbenzoic acid;
mmol is millimole;
Me is methyl;
MeCN is acetonitrile;
MeMgBr is methylmagnesium bromide;
MeMgCl is methylmagnesium chloride;
MeOH is methanol;
min is minute;
MOM is methoxymethyl;
Ms is methanesulfonyl;
MS is mass spectrometry;
MTBE is methyl tert-butyl ether;
MW is microwave;
N is normal;
NaHMDS is sodium bis(trimethylsilyl)amide;
NaOAc is sodium acetate;
NaOtBu is sodium tert-butoxide;
NBS is N-bromosuccinimide;
NCS is N-chlorosuccinimide;
NIS is N-iodosuccinimide;
NMO is N-methylmorpholine N-oxide;
NMP is N-methyl pyrrolidinone;
NMR is nuclear magnetic resonance spectroscopy;
PdCl₂(dppf) is [1,1′-Bis(diphenylphosphino)ferrocene]dichloropalladium(II);
PdCl₂(dppf)·CH₂Cl₂ is [1,1′-Bis(diphenylphosphino)ferrocene]dichloropalladium(II), complex with dichloromethane;
Pd₂(dba)₃ is tris(dibenzylideneacetone)dipalladium;
PdCl₂(PPh₃)₂ is dichlorobis-(triphenylphosphene) palladium;
Pd-PEPPSI™-SIPr is (1,3-Bis(2,6-diisopropylphenyl)imidazolidene) (3-chloropyridyl) palladium(II) dichloride;
PG Denotes a protecting group;
Ph is phenyl;
PhMe is toluene;
Ply-Cl is pivaloyl chloride, Trimethylacetyl chloride;
PPh₃ is triphenylphosphine;
PMB is para-methoxybenzyl;
Reagent alcohol is a mixture of 90% ethanol, 5% isopropanol and 5% methanol;
rt or RT is room temperature;
RBF is round-bottom flask;
RuPhos Pd G1 is chloro-(2-Dicyclohexylphosphino-2′,6′-diisopropoxy-1,1′-biphenyl)[2-(2-aminoethyl)phenyl]palladium(II);
sat. is saturated;
SEM is [2-(trimethylsilyl)ethoxy]methyl;
SFC is supercritical fluid chromatography;
SNAr is nucleophilic aromatic substitution;
TBAB is tetrabutyl ammonium bromide;
TBAF is tetrabutyl ammonium fluoride;
TBS is tert-butyldimethylsilyl;
tBu is tert-butyl;
Tf is trifluoromethanesulfonyl;
TFA is trifluoroacetic acid;
THF is tetrahydrofuran;
THP is tetrahydropyran;
TLC is thin layer chromatography;
TMAD is tetramethylazodicarboxamide;
TMS is trimethylsilyl;
TPAP is tetrapropylammonium perruthenate;
Ts is p-toluenesulfonyl;
UPLC is ultra-performance liquid chromatography;
Xantphos is 4,5-Bis(diphenylphosphino)-9,9-dimethylxanthene.

### Definitions

The term “aberrant,” as used herein, refers to different from normal. When used to describe activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g. by administering a compound or using a method as described herein), results in reduction of the disease or one or more disease symptoms.

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C1-7 alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent alkenyl group. An optionally substituted alkenylene is an alkenylene that is optionally substituted as described herein for alkenyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; alkoxy; aryl; aryloxy; azido; cycloalkyl; cycloalkoxy; cycloalkenyl; cycloalkynyl; halo; heterocyclyl;

(heterocyclyl)oxy; heteroaryl; hydroxy; nitro; thiol; silyl; cyano; alkylsulfonyl; alkylsulfinyl; alkylsulfenyl; ═O; ═S; —C(O)R or —SO₂R, where R is amino; and ═NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “alkynylene,” as used herein, refers to a divalent alkynyl group. An optionally substituted alkynylene is an alkynylene that is optionally substituted as described herein for alkynyl.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —C(O)OR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; —(CH₂)ₙ—C(O)OR^{A}; —C(O)R; and —SO₂R, where R is amino or alkyl, R^{A} is H or alkyl, and n is 0 or 1. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “azido,” as used herein, represents an —N₃ group.

The term “cancer,” as used herein, refers to all types of cancer, neoplasm or malignant tumors found in mammals (e.g., humans).

The term “carbocyclic,” as used herein, represents an optionally substituted C3-16 monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “carcinoma,” as used herein, refers to a malignant new growth made up of epithelial cells tending to infiltrate the surrounding tissues and give rise to metastases.

The term “cyano,” as used herein, represents —CN group.

The terms “CCNE1” and “cyclin E1,” as used interchangeably herein, refer to G1/S specific cyclin E1 (Gene name: CCNE1). A cell overexpressing CCNE1 is one that exhibits a higher activity of CCNE1 than a cell normally expressing CCNE1. For example, a CCNE1-overexpressing cell is a cell that exhibits a copy number of at least 3 compared to a diploid normal cell with 2 copies. Thus, a cell exhibiting a copy number greater than 3 of CCNE1 is a cell overexpressing CCNE1. The CCNE1 overexpression may be measured by identifying the expression level of the gene product in a cell (e.g., CCNE1 mRNA transcript count or CCNE1 protein level).

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, represents a divalent cycloalkenyl group. An optionally substituted cycloalkenylene is a cycloalkenylene that is optionally substituted as described herein for cycloalkyl.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C_{3-C10} cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1]heptyl, 2-bicyclo[2.2.1]heptyl, 5-bicyclo[2.2.1]heptyl, 7-bicyclo[2.2.1]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is optionally substituted amino; ═NR′, where R′ is H, alkyl, aryl, or heterocyclyl; and —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “FBXW7,” as used herein, refers to F-box/WD Repeat-Containing Protein 7 gene, transcript, or protein. An FBXW7-mutated gene, also described herein as an FBXW7 gene having an inactivating mutation, is one that fails to produce a functional FBXW7 protein or produces reduced quantities of FBXW7 protein in a cell.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms.

The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; —C(O)R or —SO₂R, where R is amino or alkyl; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic).

The term “lymphoma,” as used herein, refers to a cancer arising from cells of immune origin.

The term “melanoma,” as used herein, is taken to mean a tumor arising from the melanocytic system of the skin and other organs.

The term “Myt1,” as used herein, refers to membrane-associated tyrosine and threonine-specific cdc2-inhibitory kinase (Myt1) (Gene name PKMYT1).

The term “Myt1 inhibitor,” as used herein, represents a compound that upon contacting the enzyme Myt1, whether in vitro, in cell culture, or in an animal, reduces the activity of Myt1, such that the measured Myt1 IC₅₀ is 10 μM or less (e.g., 5 μM or less or 1 μM or less). For certain Myt1 inhibitors, the Myt1 IC₅₀ may be 100 nM or less (e.g., 10 nM or less, or 3 nM or less) and could be as low as 100 pM or 10 pM. Preferably, the Myt1 IC₅₀ is 1 nM to 1 μM (e.g., 1 nM to 750 nM, 1 nM to 500 nM, or 1 nM to 250 nM). Even more preferably, the Myt1 IC₅₀ is less than 20 nm (e.g., 1 nM to 20 nM).

The term “nitro,” as used herein, represents an —NO₂ group.

The term “oxo,” as used herein, represents a divalent oxygen atom (e.g., the structure of oxo may be shown as ═O).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pre-malignant” or “pre-cancerous,” as used herein, refers to a condition that is not malignant but is poised to become malignant.

The term “protecting group,” as used herein, represents a group intended to protect a hydroxy, an amino, or a carbonyl from participating in one or more undesirable reactions during chemical synthesis.

The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone-enol, enamine-imine, amide-imidic acid, nitroso-oxime, ketene-ynol, and amino acid-ammonium carboxylate.

The term “sarcoma” generally refers to a tumor which is made up of a substance like the embryonic connective tissue and is generally composed of closely packed cells embedded in a fibrillar or homogeneous substance.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

### BRIEF DESCRIPTION OF THE DRAWINGS

FIG. 1A is a bar graph showing the CCNE1 amplification/overexpression across tumors sequenced from TCGA PanCancer Atlas.

FIG. 1B is a scatter plot showing the CCNE1 gene expression data from TCGA PanCancer Atlas.

FIG. 2A is a bar graph showing the FBXW7 mutations across tumors sequenced fromTCGA PanCancer Atlas.

FIG. 2B is a lollipop graph showing the frequency of FBXW7 mutations across the gene. This graph highlights three common arginine hotspot mutations (R465, R479, and R505) within the third and fourth WD40 repeats that disrupt recognition of the Cyclin E1 substrate and are classified as deleterious.

FIG. 3A is a bar graph showing the results of a proliferation assay using RPE1-hTERT Cas9 TP53−/− and CCNE1-overexpressing clones treated with different doses of compound A.

FIG. 3B is a series of images depicting the results of a clonogenic survival assay using RPE1-hTERT Cas9 TP53−/− and CCNE1-overexpressing clones transduced with PKMYT1 sgRNAs. Infected cells were plated at low density to measure their ability to form colonies of >50 cells. After 10 days of growth, the colonies were stained, imaged, and quantified. The results are normalized to the survival of RPE1-hTERT Cas9 TP53−/− parental and CCNE1-overexpressing clones transduced with a non-targeting LacZ control sgRNA.

FIG. 3C is a line graph showing the results of a proliferation assay using RPE1-hTERT Cas9 TP54−/− and CCNE1-overexpressing clones treated with different doses of compound A.

FIG. 4A is a bar graph showing the results of a clonogenic survival assay using FT282-hTERT TP53^{R175H} and CCNE1-overexpressing cells transduced with PKMYT1 sgRNAs. Infected cells were plated at low density to measure their ability to form colonies of >50 cells. After 10 days of growth, the colonies were stained, imaged, and quantified. The results are normalized to the survival of FT282-hTERT TP53^{R175H} and CCNE1-overexpressing cells transduced with an AAVS1 control sgRNA.

FIG. 4B is a series of images showing of stained colonies described in FIG. 4A.

FIG. 4C is a line graph showing the results of a proliferation assay using FT282-hTERT TP53^{R175H} and CCNE1-overexpressing clones treated with different doses of compound A.

FIGS. 5A, 5B, and 5C show the results of clonogenic survival assays for stable RPE1-hTERT Cas9 TP53−/− parental and CCNE1-overexpressing clones expressing either a wild type or catalytic-dead FLAG-tagged PKMYT1 sgRNA-resistant ORF. These stable cell lines were transduced with either a LacZ non-targeting sgRNA or PKMYT1 sgRNA #4 and plated at low density to measure their ability to form colonies of >50 cells. After 10 days of growth, the colonies were stained, imaged, and quantified. The results are normalized to the survival of RPE1-hTERT Cas9 TP53−/− CCNE1-overexpressing clones transduced with a non-targeting LacZ control sgRNA and represented as a bar graph in FIG. 5C. Both clones 2 and 21 behave similarly in this study.

FIG. 6 is a chart showing the results of proliferation assays for a panel of CCNE1 wild type and CCNE1-amplified/overexpressing cancer cell lines treated with different doses of compound B. The IC₅₀ values are plotted for each cell line and demonstrate that CCNE1-overexpressing cell lines show enhanced sensitivity to a Myt1 inhibitor compared to CCNE1 WT cell lines.

FIG. 7 is a chart showing the results of proliferation assays for a panel of FBXW7 wild type and FBXW7-mutated cancer cell lines treated with different doses of compound C. The IC50 values are plotted for each cell line and demonstrate that FBXW7-mutated cell lines show enhanced sensitivity to a Myt1 inhibitor compared to FBXW7 WT cell lines.

### DETAILED DESCRIPTION

In general, the invention provides compounds, pharmaceutical compositions containing the same, methods of preparing the compounds, and methods of use. Compounds of the invention may be Myt1 inhibitors. These compounds may be used to inhibit Myt1 in a cell, e.g., a cell in a subject (e.g., a cell overexpressing CCNE1 or having an inactivating mutation in the FBXW7 gene). The subject may be in need of a treatment for a disease or condition, e.g., a disease or condition having a symptom of cell hyperproliferation, e.g., a cancer. The Myt1 inhibitory activity of the compounds disclosed herein is useful for treating a subject in need of a treatment for cancer.

Myt1 is a cell cycle regulating kinase localized predominantly in the endoplasmic reticulum and golgi complex. It is part of the Wee family of kinases that includes Wee1 and Wee1b. It is involved in the negative regulation of the CDK1-Cyclin B complex which promotes the progression of cells from G2-phase into the mitotic phase (M-phase) of the cell cycle. During DNA damage, Myt1 drives the phosphorylation on CDK1 (both Tyr15 and Thr14 of CDK1) which maintains the kinase complex in an inactive state in G2 as part of the G2 checkpoint response along with Wee1 (which mediates only Tyr15 phosphorylation) and prevents entry into mitosis until the damage has been repaired. Additionally, it has been proposed that Myt1 directly interacts with CDK1 complexes in the cytoplasm and prevents their nuclear translocation thus inhibiting cell cycle progression.

Myt1 has been implicated as a potentially important cancer target as it is essential in many cancer cells. Overexpression of Myt1 has been observed in various cancers including hepatocellular carcinoma as well as clear-cell renal-cell carcinoma. Myt1 downregulation has a minor role in unperturbed cells but has a more prominent role in cells exposed to DNA damage. Additionally, cells that exhibit high levels of replication stress in addition to defective G1 checkpoint regulation may be particularly sensitive to loss of Myt1 function, as these cells will be prone to entering mitosis prematurely with compromised genomic material leading to mitotic catastrophe.

Inhibitors of Myt1, a regulator of G2-M transition, may be particularly useful in the treatment of tumors harboring CCNE1-amplification or FBXW7 loss-of-function mutations using a synthetic lethal therapeutic strategy.

Cyclin E1 (encoded by the CCNE1 gene) is involved in the G1 to S phase cell cycle transition. In late G1 phase of the cell cycle, it complexes with cyclin-dependent kinase 2 (CDK2) to promote E2F transcription factor activation and entry into S-phase. Cyclin E1 levels are tightly regulated during normal cell cycles, accumulating at the G1/S transition and being completely degraded by the end of S phase. The cell cycle-dependent proteasomal degradation of Cyclin E1 is mediated by the SCF^{FBW7} ubiquitin ligase complex. Once activated in late G1, the Cyclin E1/CDK2 complex promotes the transition into S phase through phosphorylation and inactivation of RB1 and subsequent release of E2F transcription factors. S phase is promoted by E2F-mediated transcription of numerous genes involved in DNA replication including the pre-replication complex subunits ORC1, CDC6, CDT1, and the MCM helicase factors.

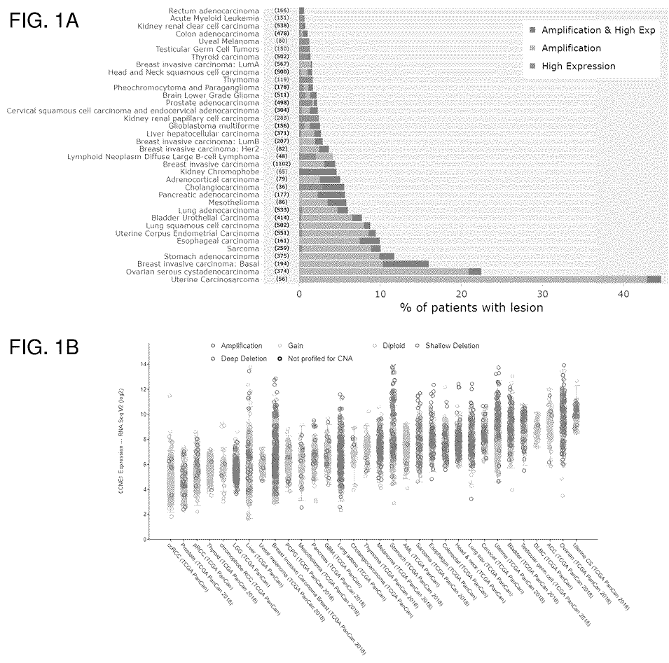

CCNE1 is frequently amplified and/or over-expressed in human cancers (FIG. 1). CCNE1 amplification has been reported in several cancer types including endometrial, ovarian, breast and gastric, ranging in frequency from 5-40%. Importantly, numerous studies have confirmed Cyclin E1 as a driver of tumorigenesis in these indications and CCNE1 amplification is observed in the more aggressive subtypes including uterine carcinosarcoma (UCS; ˜40%), uterine serous carcinoma (USC; ˜25%), high-grade serous ovarian carcinoma (HGSOC; ˜25%), and triple-negative breast cancer (TNBC; ˜8%). Patients with evidence of Cyclin E1 over-expression in tumor biopsies by immunohistochemistry and/or genomic copy number analysis have a lower overall survival compared to patients with normal Cyclin E1 levels. HGSOC patients with Cyclin E1 over-expression have a lower response rate to cisplatin, the current standard of care.

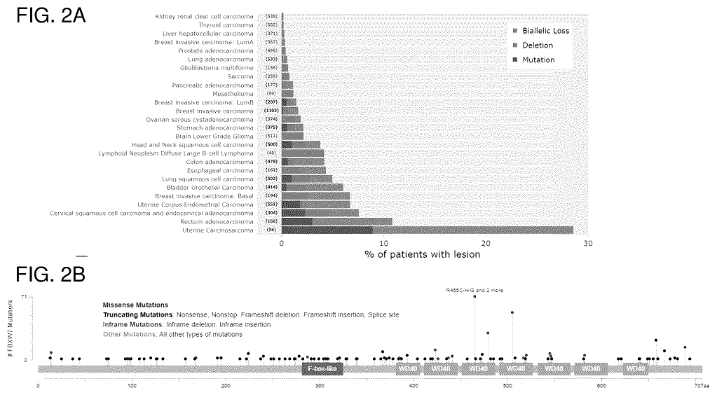

Defective cell cycle-regulated proteolysis of Cyclin E1 by the SCF^{FBW7} ubiquitin ligase complex is another mechanism of CCNE1 over-expression observed in tumors. The F-box protein gene, FBXW7, is frequently mutated in several cancer types including endometrial, colorectal, and gastric, ranging in frequency from 5-35% (FIG. 2). Like CCNE1, FBXW7 driver mutations are observed in the more aggressive subtypes of endometrial cancer including UCS (˜35%) and USC (˜25%). FBXW7 has a diverse spectrum of loss-of-function mutations in cancer including truncating mutations peppered across the gene and missense mutations within the Cyclin E1 recognizing WD40 repeats. FBW7 functions as a homodimer within the SCF complex and many deleterious missense mutations within the WD40 repeats are mostly heterozygous and dominant negative. Remarkably, several recurring hotspot missense mutations are found in the WD40 repeats including R465, R479, and R505—all of which disrupt Cyclin E1 binding and ubiquitylation.

Cyclin E1 over-expression and/or FBXW7 loss-of-function is thought to drive tumorigenesis by inducing genome instability (e.g., increased origin firing, defective nucleotide pools, transcription-replication conflicts, and/or fork instability). Over-expression of Cyclin E1 has been shown to induce replication stress characterized by slowed or stalled replication forks and loss-of-heterozygosity at fragile sites. The primary mechanism by which Cyclin E1 over-expression causes replication stress is increased origin firing in early S-phase followed by depletion of replication factors including nucleotide pools. The decrease in overall replication proteins and nucleotides decreases fork progression and causes stalling and subsequent collapse or reversal.

The compound of the invention may be, e.g., a compound of formula (I):

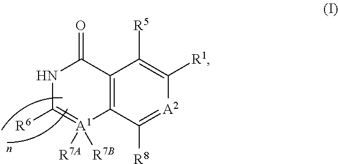

or a pharmaceutically acceptable salt thereof,
where

R¹ is:

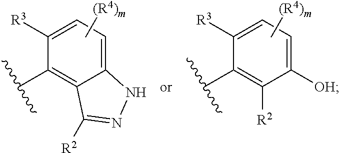

[symbol] is a single bond, and n is 0 or 1; or [symbol] is a double bond, and n is 1;
m is 0, 1, or 2;
A¹ is N, R^{7A} is absent, hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂, and R^{7B} is absent; or A¹ is C, and R^{7A} is hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂, and R^{7B} is absent, hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂;
A² is N or CH;
each of R² and R³ are independently hydrogen, halogen, optionally substituted C₁₋₆ alkyl, or optionally substituted C₃₋₈ cycloalkyl;
each R⁴ is independently halogen;
R⁵ is hydrogen, halogen, hydroxyl, optionally substituted C₁₋₆ alkyl, —N(R^{5A})₂, or —OR^{5B};
each R^{5A} is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl; or two R^{5A} groups, together with the atom to which both are attached, combine to form an optionally substituted C₂₋₉ heterocyclyl;
R^{5B} is optionally substituted C₁₋₆ alkyl;
R⁶ is hydrogen, halogen, optionally substituted C₁₋₆ alkyl, optionally substituted C₁₋₆ heteroalkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{6A}, —N(R^{6B})₂, or -L¹-R^{6C};
R^{6A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{6B} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{6C} is hydrogen, halogen, optionally substituted C₁₋₆ alkyl, optionally substituted C₂₋₆ alkenyl, optionally substituted C₂₋₆ alkynyl, optionally substituted acyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{6D}, or —N(R⁶⁹²;
R^{6D} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{6E} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{7C} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{7D} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R⁸ is hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{8A}, or -L²-R^{8B};
R^{8A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{8B} is hydrogen, halogen, cyano, optionally substituted C₁₋₆ alkyl, optionally substituted C₂₋₆ alkenyl, optionally substituted C₂₋₆ alkynyl, optionally substituted acyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{8C}, —N(R^{8D})₂, —CO₂H, —NHC(O)(R^{8E}), —C(O)N(R^{8E})₂;
R^{8C} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{8D} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{8E} is independently hydrogen, optionally substituted C₁₋₆ alkyl, optionally substituted C₁₋₆ heteroalkyl, optionally substituted C₃₋₈ cycloalkyl, or optionally substituted C₂₋₉ heterocyclyl; or two R^{8E} groups, together with the atom to which both are attached, combine to form an optionally substituted C₂₋₉ heterocyclyl; and
each of L¹ and L² is independently optionally substituted C₂₋₉ heterocyclylene, optionally substituted C₂₋₉ heteroarylene, optionally substituted C₆₋₁₀ arylene, or optionally substituted C₃₋₈ cycloalkylene.

The compound of the invention may be, e.g., a compound listed in Table 1 below or a pharmaceutically acceptable salt thereof.

TABLE 1

| | |
|---|---|
| 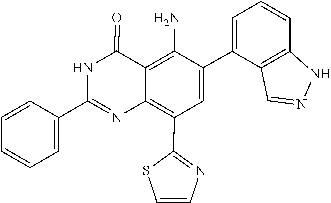 [SMILES: Nc1c(-c2cccc3[nH]ncc23)cc(-c2nccs2)c2nc(-c3ccccc3)[nH]c(=O)c12] | 1 |
| 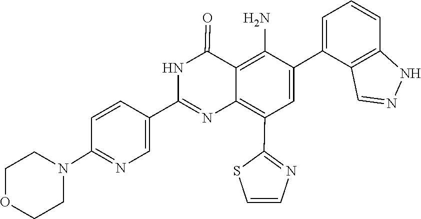 [SMILES: Nc1c(-c2cccc3[nH]ncc23)cc(-c2nccs2)c2nc(-c3ccc(N4CCOCC4)nc3)[nH]c(=O)c12] | 2 |
| 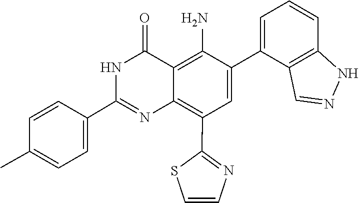 [SMILES: Cc1ccc(-c2nc3c(-c4nccs4)cc(-c4cccc5[nH]ncc45)c(N)c3c(=O)[nH]2)cc1] | 3 |
| 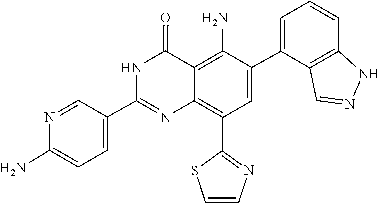 [SMILES: Nc1ccc(-c2nc3c(-c4nccs4)cc(-c4cccc5[nH]ncc45)c(N)c3c(=O)[nH]2)cn1] | 4 |
| 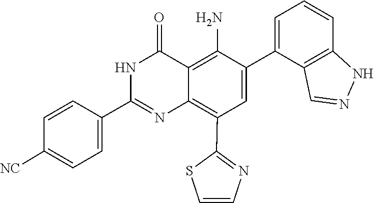 [SMILES: N#Cc1ccc(-c2nc3c(-c4nccs4)cc(-c4cccc5[nH]ncc45)c(N)c3c(=O)[nH]2)cc1] | 5 |
| 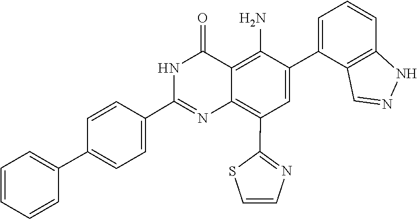 [SMILES: Nc1c(-c2cccc3[nH]ncc23)cc(-c2nccs2)c2nc(-c3ccc(-c4ccccc4)cc3)[nH]c(=O)c12] | 6 |
| 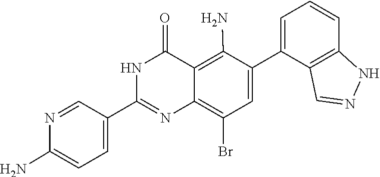 [SMILES: Nc1ccc(-c2nc3c(Br)cc(-c4cccc5[nH]ncc45)c(N)c3c(=O)[nH]2)cn1] | 7 |
| 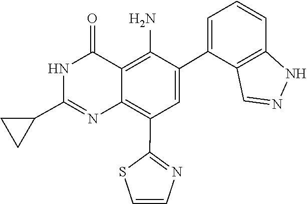 [SMILES: Nc1c(-c2cccc3[nH]ncc23)cc(-c2nccs2)c2nc(C3CC3)[nH]c(=O)c12] | 8 |
| 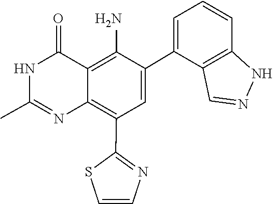 [SMILES: Cc1nc2c(-c3nccs3)cc(-c3cccc4[nH]ncc34)c(N)c2c(=O)[nH]1] | 9 |
| 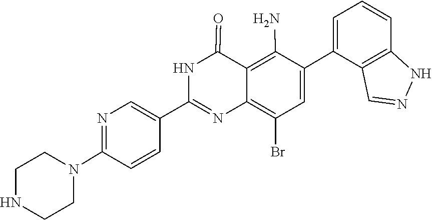 [SMILES: Nc1c(-c2cccc3[nH]ncc23)cc(Br)c2nc(-c3ccc(N4CCNCC4)nc3)[nH]c(=O)c12] | 10 |
| 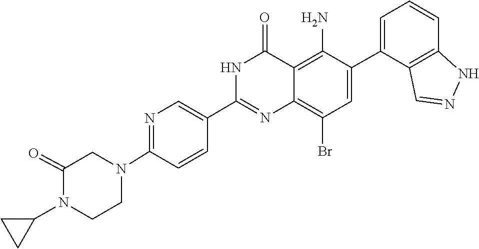 [SMILES: Nc1c(-c2cccc3[nH]ncc23)cc(Br)c2nc(-c3ccc(N4CCN(C5CC5)C(=O)C4)nc3)[nH]c(=O)c12] | 11 |
| 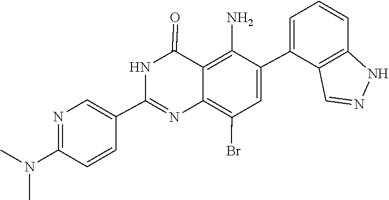 [SMILES: CN(C)c1ccc(-c2nc3c(Br)cc(-c4cccc5[nH]ncc45)c(N)c3c(=O)[nH]2)cn1] | 12 |
| 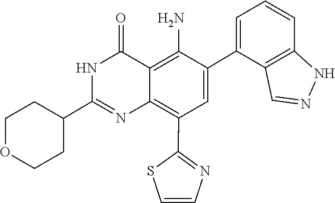 [SMILES: Nc1c(-c2cccc3[nH]ncc23)cc(-c2nccs2)c2nc(C3CCOCC3)[nH]c(=O)c12] | 13 |
| 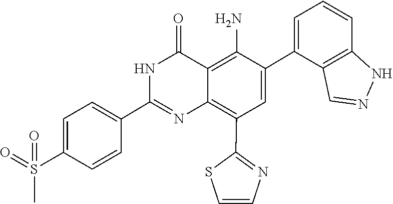 [SMILES: CS(=O)(=O)c1ccc(-c2nc3c(-c4nccs4)cc(-c4cccc5[nH]ncc45)c(N)c3c(=O)[nH]2)cc1] | 14 |
| 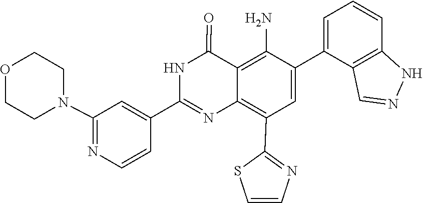 [SMILES: Nc1c(-c2cccc3[nH]ncc23)cc(-c2nccs2)c2nc(-c3ccnc(N4CCOCC4)c3)[nH]c(=O)c12] | 15 |
| 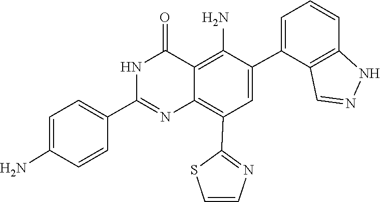 [SMILES: Nc1ccc(-c2nc3c(-c4nccs4)cc(-c4cccc5[nH]ncc45)c(N)c3c(=O)[nH]2)cc1] | 16 |
| 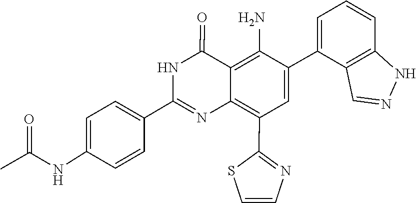 [SMILES: CC(=O)Nc1ccc(-c2nc3c(-c4nccs4)cc(-c4cccc5[nH]ncc45)c(N)c3c(=O)[nH]2)cc1] | 17 |
| 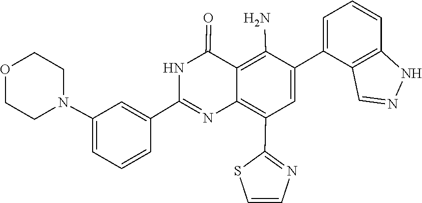 [SMILES: Nc1c(-c2cccc3[nH]ncc23)cc(-c2nccs2)c2nc(-c3cccc(N4CCOCC4)c3)[nH]c(=O)c12] | 18 |
| 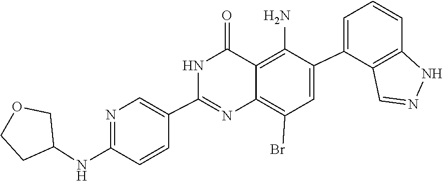 [SMILES: Nc1c(-c2cccc3[nH]ncc23)cc(Br)c2nc(-c3ccc(NC4CCOC4)nc3)[nH]c(=O)c12] | 19 |
| 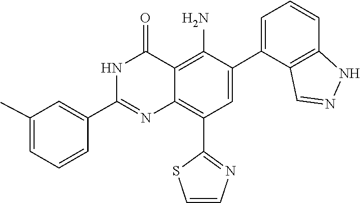 [SMILES: Cc1cccc(-c2nc3c(-c4nccs4)cc(-c4cccc5[nH]ncc45)c(N)c3c(=O)[nH]2)c1] | 20 |
| 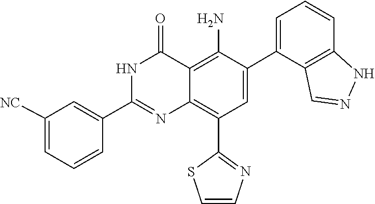 [SMILES: N#Cc1cccc(-c2nc3c(-c4nccs4)cc(-c4cccc5[nH]ncc45)c(N)c3c(=O)[nH]2)c1] | 21 |
| 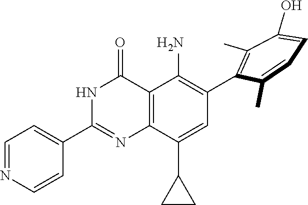 | 22 |
| 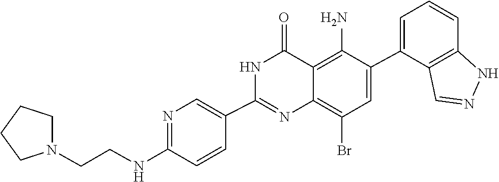 [SMILES: Nc1c(-c2cccc3[nH]ncc23)cc(Br)c2nc(-c3ccc(NCCN4CCCC4)nc3)[nH]c(=O)c12] | 23 |
| 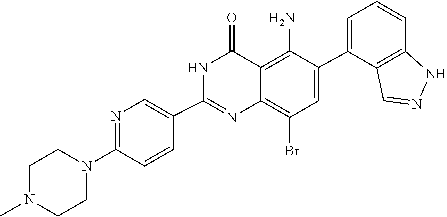 [SMILES: CN1CCN(c2ccc(-c3nc4c(Br)cc(-c5cccc6[nH]ncc56)c(N)c4c(=O)[nH]3)cn2)CC1] | 24 |
| 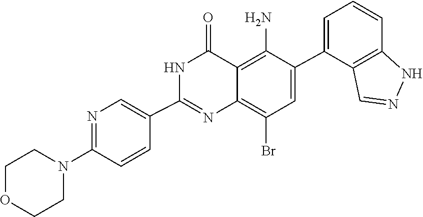 [SMILES: Nc1c(-c2cccc3[nH]ncc23)cc(Br)c2nc(-c3ccc(N4CCOCC4)nc3)[nH]c(=O)c12] | 25 |
| 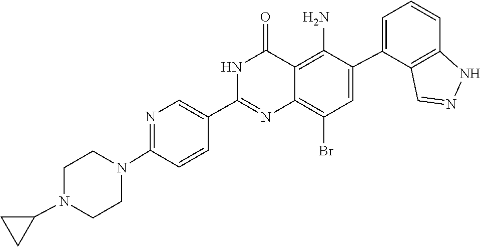 [SMILES: Nc1c(-c2cccc3[nH]ncc23)cc(Br)c2nc(-c3ccc(N4CCN(C5CC5)CC4)nc3)[nH]c(=O)c12] | 26 |
| 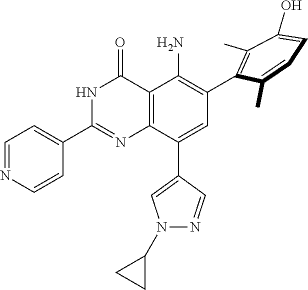 | 27 |
| 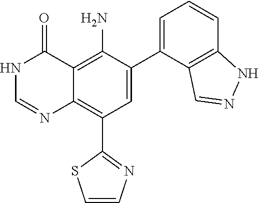 [SMILES: Nc1c(-c2cccc3[nH]ncc23)cc(-c2nccs2)c2nc[nH]c(=O)c12] | 28 |
| 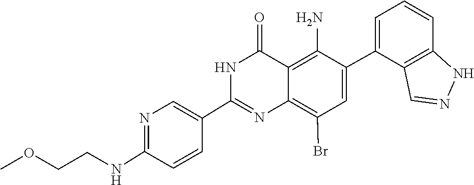 [SMILES: COCCNc1ccc(-c2nc3c(Br)cc(-c4cccc5[nH]ncc45)c(N)c3c(=O)[nH]2)cn1] | 29 |
| 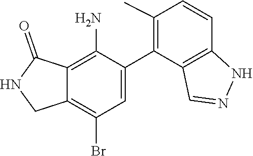 [SMILES: Cc1ccc2[nH]ncc2c1-c1cc(Br)c2c(c1N)C(=O)NC2] | 30 |
| 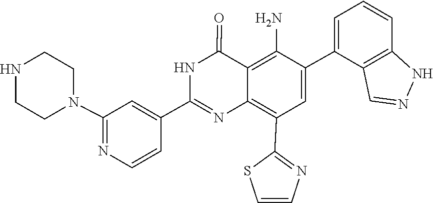 [SMILES: Nc1c(-c2cccc3[nH]ncc23)cc(-c2nccs2)c2nc(-c3ccnc(N4CCNCC4)c3)[nH]c(=O)c12] | 31 |
| 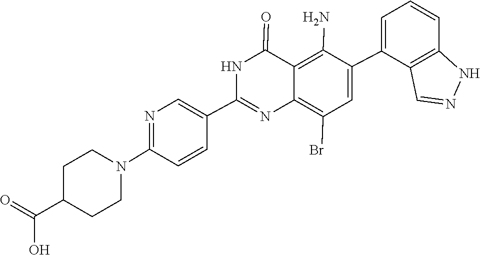 [SMILES: Nc1c(-c2cccc3[nH]ncc23)cc(Br)c2nc(-c3ccc(N4CCC(C(=O)O)CC4)nc3)[nH]c(=O)c12] | 32 |
| 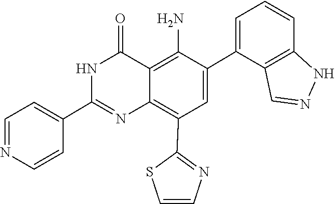 [SMILES: Nc1c(-c2cccc3[nH]ncc23)cc(-c2nccs2)c2nc(-c3ccncc3)[nH]c(=O)c12] | 33 |
| 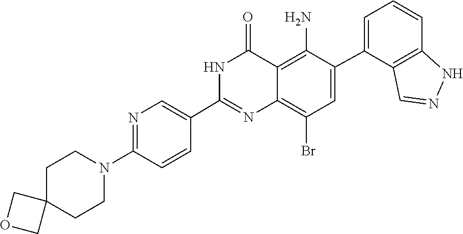 [SMILES: Nc1c(-c2cccc3[nH]ncc23)cc(Br)c2nc(-c3ccc(N4CCC5(CC4)COC5)nc3)[nH]c(=O)c12] | 34 |
| 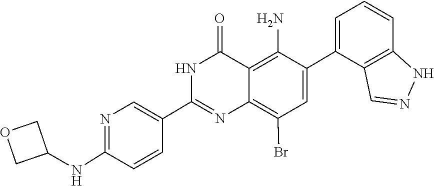 [SMILES: Nc1c(-c2cccc3[nH]ncc23)cc(Br)c2nc(-c3ccc(NC4COC4)nc3)[nH]c(=O)c12] | 35 |
| 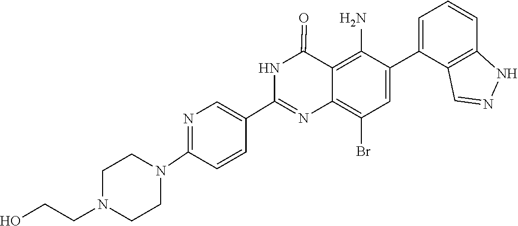 [SMILES: Nc1c(-c2cccc3[nH]ncc23)cc(Br)c2nc(-c3ccc(N4CCN(CCO)CC4)nc3)[nH]c(=O)c12] | 36 |
| 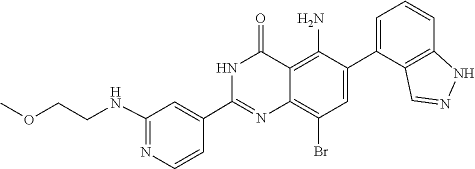 [SMILES: COCCNc1cc(-c2nc3c(Br)cc(-c4cccc5[nH]ncc45)c(N)c3c(=O)[nH]2)ccn1] | 37 |
| 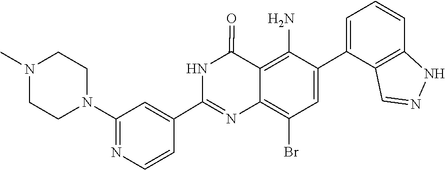 [SMILES: CN1CCN(c2cc(-c3nc4c(Br)cc(-c5cccc6[nH]ncc56)c(N)c4c(=O)[nH]3)ccn2)CC1] | 38 |
| 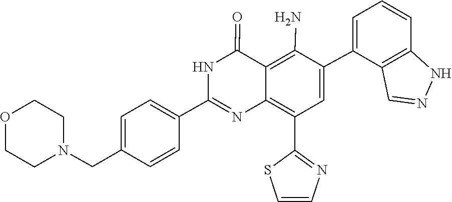 [SMILES: Nc1c(-c2cccc3[nH]ncc23)cc(-c2nccs2)c2nc(-c3ccc(CN4CCOCC4)cc3)[nH]c(=O)c12] | 39 |
| 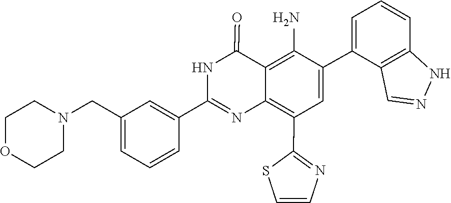 [SMILES: Nc1c(-c2cccc3[nH]ncc23)cc(-c2nccs2)c2nc(-c3cccc(CN4CCOCC4)c3)[nH]c(=O)c12] | 40 |
| 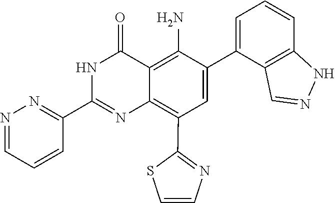 [SMILES: Nc1c(-c2cccc3[nH]ncc23)cc(-c2nccs2)c2nc(-c3cccnn3)[nH]c(=O)c12] | 41 |
| 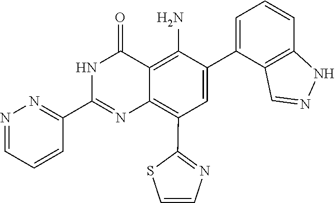 [SMILES: Nc1c(-c2cccc3[nH]ncc23)cc(-c2nccs2)c2nc(-c3cccnn3)[nH]c(=O)c12] | 42 |
| 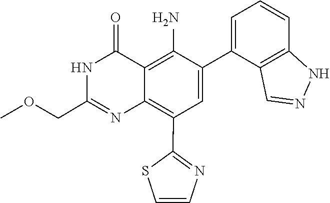 [SMILES: COCc1nc2c(-c3nccs3)cc(-c3cccc4[nH]ncc34)c(N)c2c(=O)[nH]1] | 43 |
| 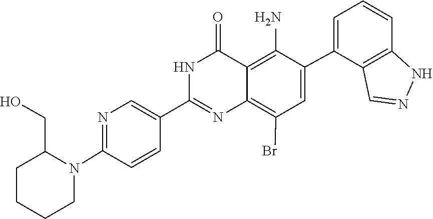 [SMILES: Nc1c(-c2cccc3[nH]ncc23)cc(Br)c2nc(-c3ccc(N4CCCCC4CO)nc3)[nH]c(=O)c12] | 44 |
| 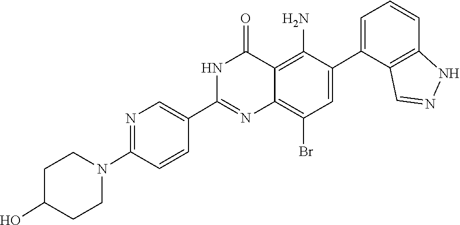 [SMILES: Nc1c(-c2cccc3[nH]ncc23)cc(Br)c2nc(-c3ccc(N4CCC(O)CC4)nc3)[nH]c(=O)c12] | 45 |
| 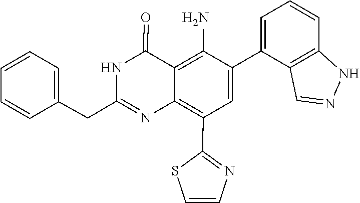 [SMILES: Nc1c(-c2cccc3[nH]ncc23)cc(-c2nccs2)c2nc(Cc3ccccc3)[nH]c(=O)c12] | 46 |
| 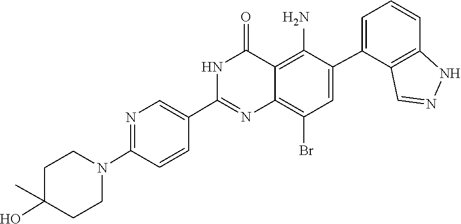 [SMILES: CC1(O)CCN(c2ccc(-c3nc4c(Br)cc(-c5cccc6[nH]ncc56)c(N)c4c(=O)[nH]3)cn2)CC1] | 47 |
| 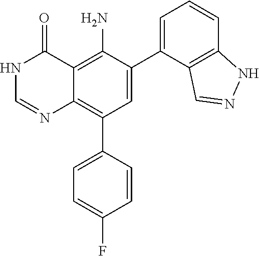 [SMILES: Nc1c(-c2cccc3[nH]ncc23)cc(-c2ccc(F)cc2)c2nc[nH]c(=O)c12] | 48 |
| 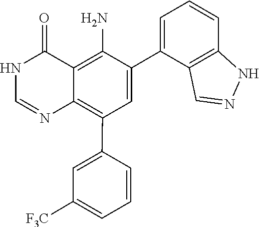 [SMILES: Nc1c(-c2cccc3[nH]ncc23)cc(-c2cccc(C(F)(F)F)c2)c2nc[nH]c(=O)c12] | 49 |
| 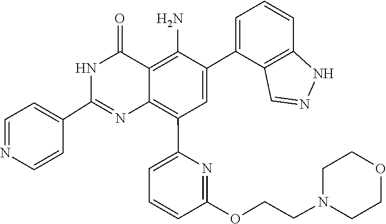 [SMILES: Nc1c(-c2cccc3[nH]ncc23)cc(-c2cccc(OCCN3CCOCC3)n2)c2nc(-c3ccncc3)[nH]c(=O)c12] | 50 |
| 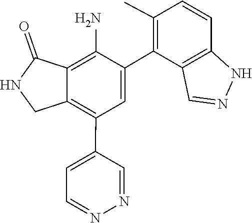 [SMILES: Cc1ccc2[nH]ncc2c1-c1cc(-c2ccnnc2)c2c(c1N)C(=O)NC2] | 51 |
| 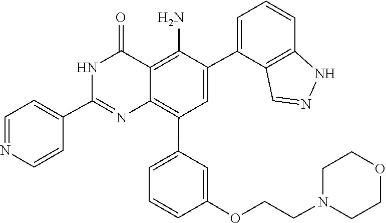 [SMILES: Nc1c(-c2cccc3[nH]ncc23)cc(-c2cccc(OCCN3CCOCC3)c2)c2nc(-c3ccncc3)[nH]c(=O)c12] | 52 |
| 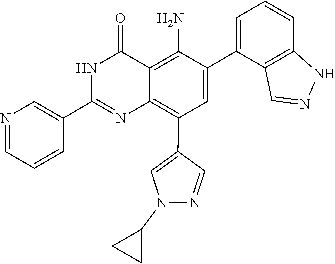 [SMILES: Nc1c(-c2cccc3[nH]ncc23)cc(-c2cnn(C3CC3)c2)c2nc(-c3cccnc3)[nH]c(=O)c12] | 53 |
| 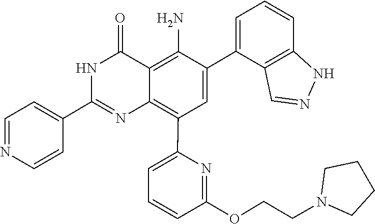 [SMILES: Nc1c(-c2cccc3[nH]ncc23)cc(-c2cccc(OCCN3CCCC3)n2)c2nc(-c3ccncc3)[nH]c(=O)c12] | 54 |
| 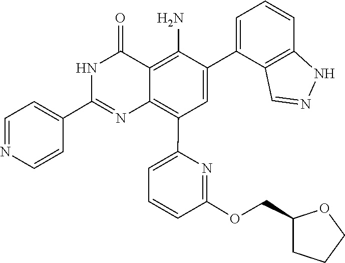 [SMILES: Nc1c(-c2cccc3[nH]ncc23)cc(-c2cccc(OC[C@@H]3CCCO3)n2)c2nc(-c3ccncc3)[nH]c(=O)c12] | 55 |
| 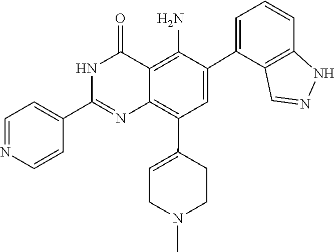 [SMILES: CN1CC=C(c2cc(-c3cccc4[nH]ncc34)c(N)c3c(=O)[nH]c(-c4ccncc4)nc23)CC1] | 56 |
| 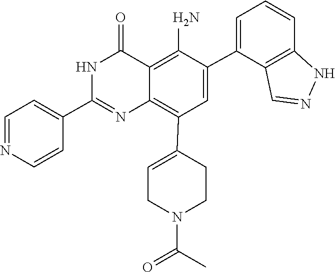 [SMILES: CC(=O)N1CC=C(c2cc(-c3cccc4[nH]ncc34)c(N)c3c(=O)[nH]c(-c4ccncc4)nc23)CC1] | 57 |
| 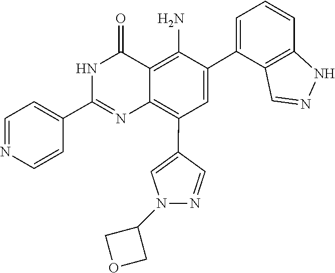 [SMILES: Nc1c(-c2cccc3[nH]ncc23)cc(-c2cnn(C3COC3)c2)c2nc(-c3ccncc3)[nH]c(=O)c12] | 58 |
| 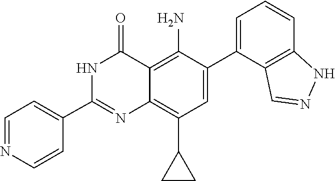 [SMILES: Nc1c(-c2cccc3[nH]ncc23)cc(C2CC2)c2nc(-c3ccncc3)[nH]c(=O)c12] | 59 |
| 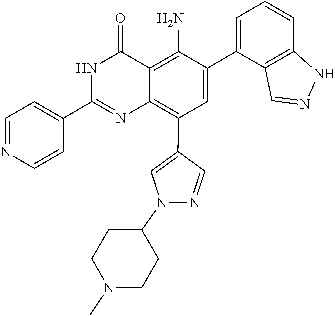 [SMILES: CN1CCC(n2cc(-c3cc(-c4cccc5[nH]ncc45)c(N)c4c(=O)[nH]c(-c5ccncc5)nc34)cn2)CC1] | 60 |
| 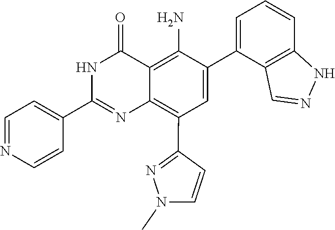 [SMILES: Cn1ccc(-c2cc(-c3cccc4[nH]ncc34)c(N)c3c(=O)[nH]c(-c4ccncc4)nc23)n1] | 61 |
| 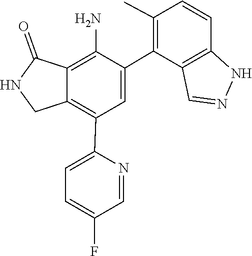 [SMILES: Cc1ccc2[nH]ncc2c1-c1cc(-c2ccc(F)cn2)c2c(c1N)C(=O)NC2] | 62 |
| 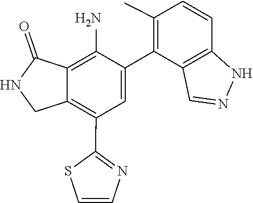 [SMILES: Cc1ccc2[nH]ncc2c1-c1cc(-c2nccs2)c2c(c1N)C(=O)NC2] | 63 |
| 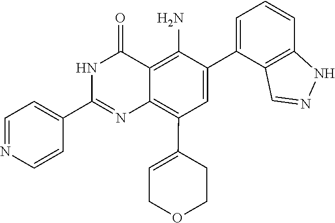 [SMILES: Nc1c(-c2cccc3[nH]ncc23)cc(C2=CCOCC2)c2nc(-c3ccncc3)[nH]c(=O)c12] | 64 |
| 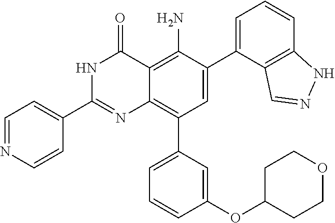 [SMILES: Nc1c(-c2cccc3[nH]ncc23)cc(-c2cccc(OC3CCOCC3)c2)c2nc(-c3ccncc3)[nH]c(=O)c12] | 65 |
| 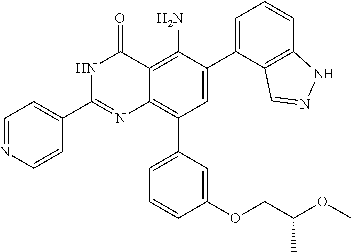 [SMILES: CO[C@H](C)COc1cccc(-c2cc(-c3cccc4[nH]ncc34)c(N)c3c(=O)[nH]c(-c4ccncc4)nc23)c1] | 66 |
| 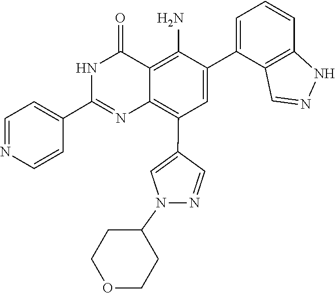 [SMILES: Nc1c(-c2cccc3[nH]ncc23)cc(-c2cnn(C3CCOCC3)c2)c2nc(-c3ccncc3)[nH]c(=O)c12] | 67 |
| 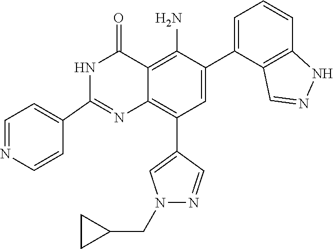 [SMILES: Nc1c(-c2cccc3[nH]ncc23)cc(-c2cnn(CC3CC3)c2)c2nc(-c3ccncc3)[nH]c(=O)c12] | 68 |
| 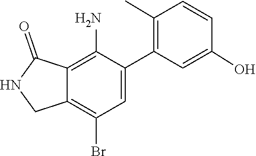 [SMILES: Cc1ccc(O)cc1-c1cc(Br)c2c(c1N)C(=O)NC2] | 69 |
| 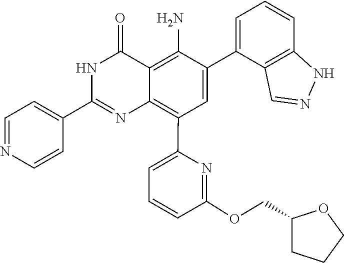 [SMILES: Nc1c(-c2cccc3[nH]ncc23)cc(-c2cccc(OC[C@H]3CCCO3)n2)c2nc(-c3ccncc3)[nH]c(=O)c12] | 70 |
| 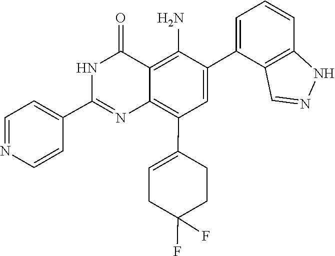 [SMILES: Nc1c(-c2cccc3[nH]ncc23)cc(C2=CCC(F)(F)CC2)c2nc(-c3ccncc3)[nH]c(=O)c12] | 71 |
| 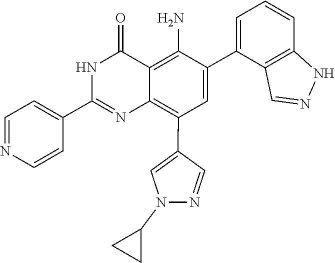 [SMILES: Nc1c(-c2cccc3[nH]ncc23)cc(-c2cnn(C3CC3)c2)c2nc(-c3ccncc3)[nH]c(=O)c12] | 72 |
| 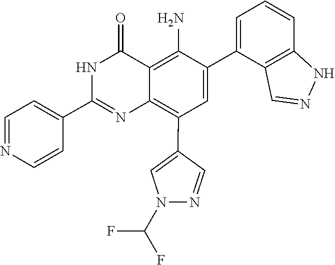 [SMILES: Nc1c(-c2cccc3[nH]ncc23)cc(-c2cnn(C(F)F)c2)c2nc(-c3ccncc3)[nH]c(=O)c12] | 73 |
| 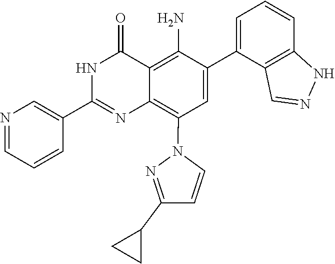 [SMILES: Nc1c(-c2cccc3[nH]ncc23)cc(-n2ccc(C3CC3)n2)c2nc(-c3cccnc3)[nH]c(=O)c12] | 74 |
| 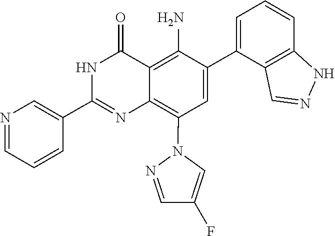 [SMILES: Nc1c(-c2cccc3[nH]ncc23)cc(-n2cc(F)cn2)c2nc(-c3cccnc3)[nH]c(=O)c12] | 75 |
| 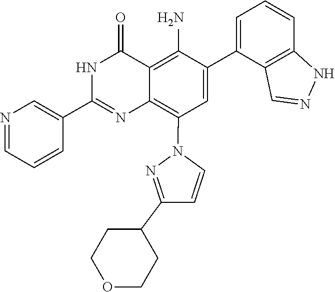 [SMILES: Nc1c(-c2cccc3[nH]ncc23)cc(-n2ccc(C3CCOCC3)n2)c2nc(-c3cccnc3)[nH]c(=O)c12] | 76 |
| 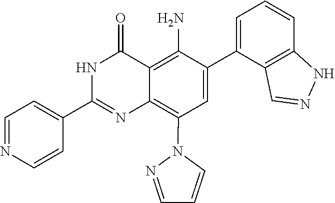 [SMILES: Nc1c(-c2cccc3[nH]ncc23)cc(-n2cccn2)c2nc(-c3ccncc3)[nH]c(=O)c12] | 77 |
| 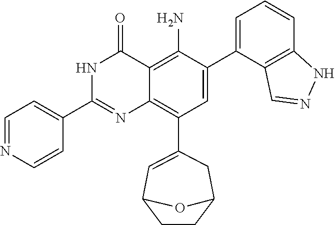 [SMILES: Nc1c(-c2cccc3[nH]ncc23)cc(C2=CC3CCC(C2)O3)c2nc(-c3ccncc3)[nH]c(=O)c12] | 78 |
| 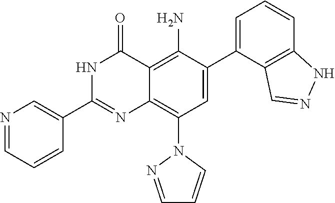 [SMILES: Nc1c(-c2cccc3[nH]ncc23)cc(-n2cccn2)c2nc(-c3cccnc3)[nH]c(=O)c12] | 79 |
| 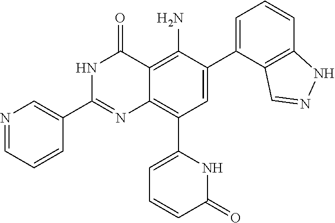 [SMILES: Nc1c(-c2cccc3[nH]ncc23)cc(-c2cccc(=O)[nH]2)c2nc(-c3cccnc3)[nH]c(=O)c12] | 80 |
| 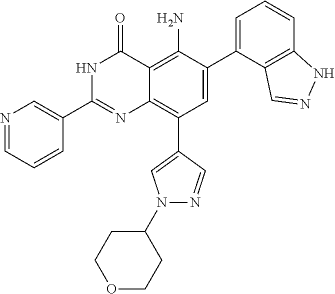 [SMILES: Nc1c(-c2cccc3[nH]ncc23)cc(-c2cnn(C3CCOCC3)c2)c2nc(-c3cccnc3)[nH]c(=O)c12] | 81 |
| 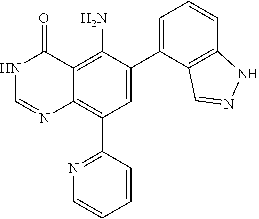 [SMILES: Nc1c(-c2cccc3[nH]ncc23)cc(-c2ccccn2)c2nc[nH]c(=O)c12] | 82 |
| 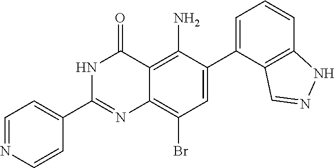 [SMILES: Nc1c(-c2cccc3[nH]ncc23)cc(Br)c2nc(-c3ccncc3)[nH]c(=O)c12] | 83 |
| 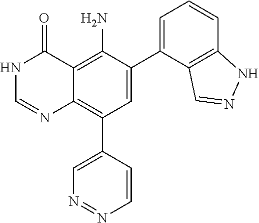 [SMILES: Nc1c(-c2cccc3[nH]ncc23)cc(-c2ccnnc2)c2nc[nH]c(=O)c12] | 84 |
| 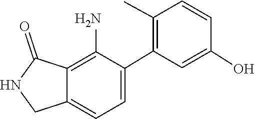 [SMILES: Cc1ccc(O)cc1-c1ccc2c(c1N)C(=O)NC2] | 85 |
| 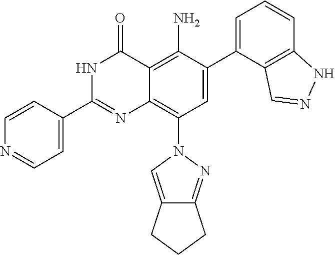 [SMILES: Nc1c(-c2cccc3[nH]ncc23)cc(-n2cc3c(n2)CCC3)c2nc(-c3ccncc3)[nH]c(=O)c12] | 86 |
| 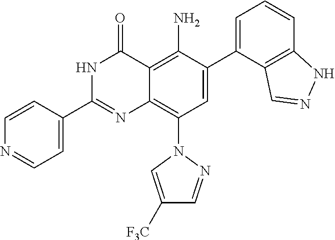 [SMILES: Nc1c(-c2cccc3[nH]ncc23)cc(-n2cc(C(F)(F)F)cn2)c2nc(-c3ccncc3)[nH]c(=O)c12] | 87 |
| 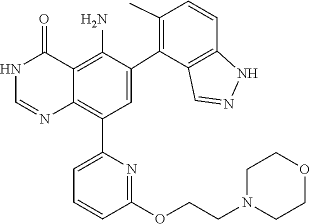 [SMILES: Cc1ccc2[nH]ncc2c1-c1cc(-c2cccc(OCCN3CCOCC3)n2)c2nc[nH]c(=O)c2c1N] | 88 |
| 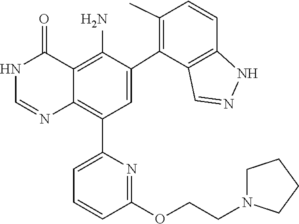 [SMILES: Cc1ccc2[nH]ncc2c1-c1cc(-c2cccc(OCCN3CCCC3)n2)c2nc[nH]c(=O)c2c1N] | 89 |
| 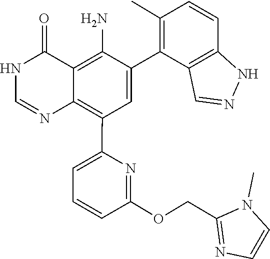 [SMILES: Cc1ccc2[nH]ncc2c1-c1cc(-c2cccc(OCc3nccn3C)n2)c2nc[nH]c(=O)c2c1N] | 90 |
| 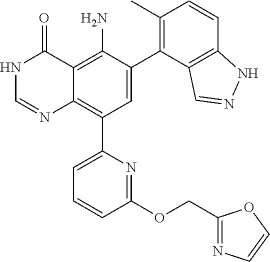 [SMILES: Cc1ccc2[nH]ncc2c1-c1cc(-c2cccc(OCc3ncco3)n2)c2nc[nH]c(=O)c2c1N] | 91 |
| 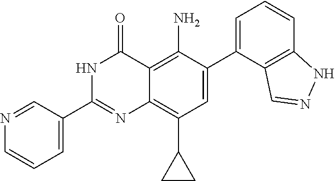 [SMILES: Nc1c(-c2cccc3[nH]ncc23)cc(C2CC2)c2nc(-c3cccnc3)[nH]c(=O)c12] | 92 |
| 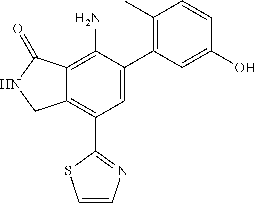 [SMILES: Cc1ccc(O)cc1-c1cc(-c2nccs2)c2c(c1N)C(=O)NC2] | 93 |
| 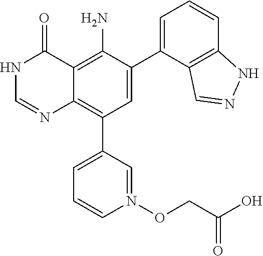 | 94 |
| 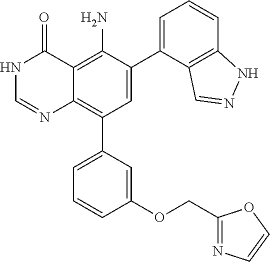 [SMILES: Nc1c(-c2cccc3[nH]ncc23)cc(-c2cccc(OCc3ncco3)c2)c2nc[nH]c(=O)c12] | 95 |
| 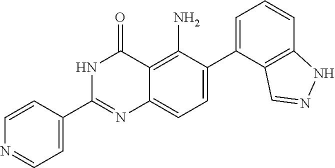 [SMILES: Nc1c(-c2cccc3[nH]ncc23)ccc2nc(-c3ccncc3)[nH]c(=O)c12] | 96 |
| 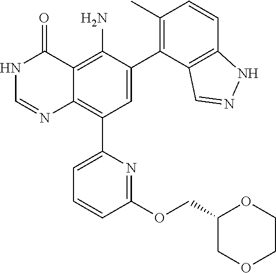 [SMILES: Cc1ccc2[nH]ncc2c1-c1cc(-c2cccc(OC[C@H]3COCCO3)n2)c2nc[nH]c(=O)c2c1N] | 97 |
| 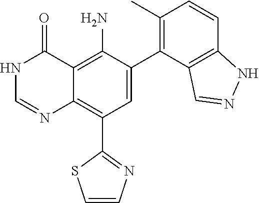 [SMILES: Cc1ccc2[nH]ncc2c1-c1cc(-c2nccs2)c2nc[nH]c(=O)c2c1N] | 98 |
| 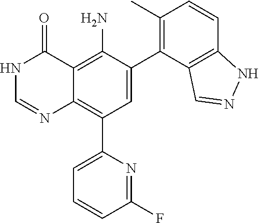 [SMILES: Cc1ccc2[nH]ncc2c1-c1cc(-c2cccc(F)n2)c2nc[nH]c(=O)c2c1N] | 99 |
| 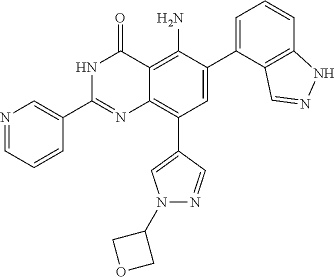 [SMILES: Nc1c(-c2cccc3[nH]ncc23)cc(-c2cnn(C3COC3)c2)c2nc(-c3cccnc3)[nH]c(=O)c12] | 100 |
| 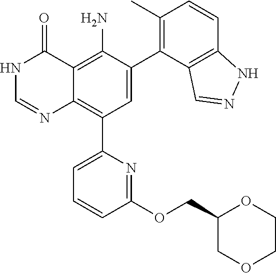 [SMILES: Cc1ccc2[nH]ncc2c1-c1cc(-c2cccc(OC[C@@H]3COCCO3)n2)c2nc[nH]c(=O)c2c1N] | 101 |
| 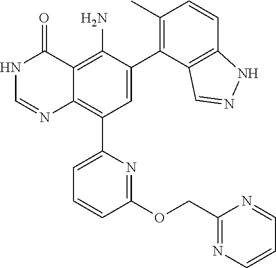 [SMILES: Cc1ccc2[nH]ncc2c1-c1cc(-c2cccc(OCc3ncccn3)n2)c2nc[nH]c(=O)c2c1N] | 102 |
| 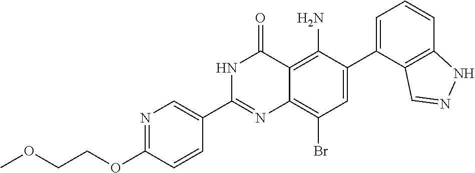 [SMILES: COCCOc1ccc(-c2nc3c(Br)cc(-c4cccc5[nH]ncc45)c(N)c3c(=O)[nH]2)cn1] | 103 |
| 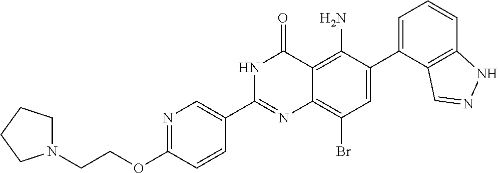 [SMILES: Nc1c(-c2cccc3[nH]ncc23)cc(Br)c2nc(-c3ccc(OCCN4CCCC4)nc3)[nH]c(=O)c12] | 104 |
| 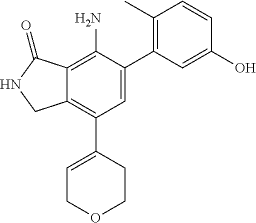 [SMILES: Cc1ccc(O)cc1-c1cc(C2=CCOCC2)c2c(c1N)C(=O)NC2] | 105 |
| 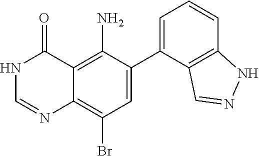 [SMILES: Nc1c(-c2cccc3[nH]ncc23)cc(Br)c2nc[nH]c(=O)c12] | 106 |
| 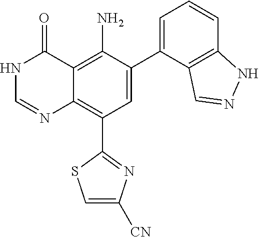 [SMILES: N#Cc1csc(-c2cc(-c3cccc4[nH]ncc34)c(N)c3c(=O)[nH]cnc23)n1] | 107 |
| 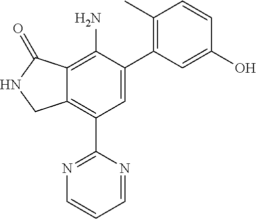 [SMILES: Cc1ccc(O)cc1-c1cc(-c2ncccn2)c2c(c1N)C(=O)NC2] | 108 |
| 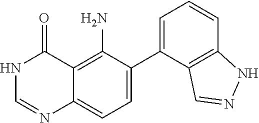 [SMILES: Nc1c(-c2cccc3[nH]ncc23)ccc2nc[nH]c(=O)c12] | 109 |
| 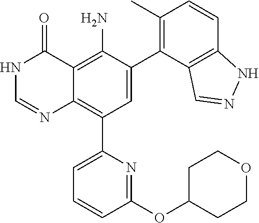 [SMILES: Cc1ccc2[nH]ncc2c1-c1cc(-c2cccc(OC3CCOCC3)n2)c2nc[nH]c(=O)c2c1N] | 110 |
| 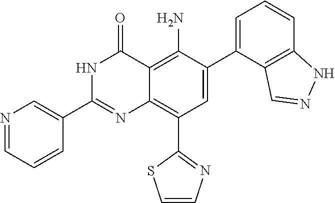 [SMILES: Nc1c(-c2cccc3[nH]ncc23)cc(-c2nccs2)c2nc(-c3cccnc3)[nH]c(=O)c12] | 111 |
| 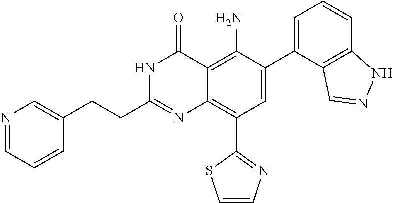 [SMILES: Nc1c(-c2cccc3[nH]ncc23)cc(-c2nccs2)c2nc(CCc3cccnc3)[nH]c(=O)c12] | 112 |
| 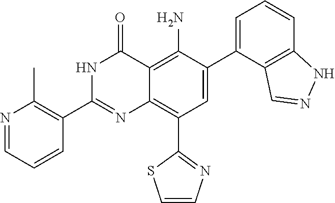 [SMILES: Cc1ncccc1-c1nc2c(-c3nccs3)cc(-c3cccc4[nH]ncc34)c(N)c2c(=O)[nH]1] | 113 |
| 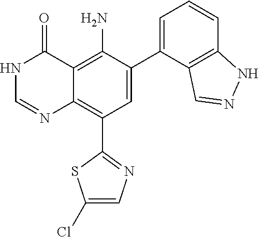 [SMILES: Nc1c(-c2cccc3[nH]ncc23)cc(-c2ncc(Cl)s2)c2nc[nH]c(=O)c12] | 114 |
| 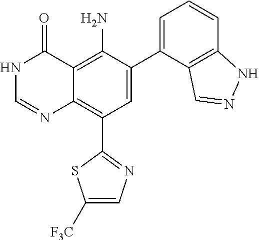 [SMILES: Nc1c(-c2cccc3[nH]ncc23)cc(-c2ncc(C(F)(F)F)s2)c2nc[nH]c(=O)c12] | 115 |
| 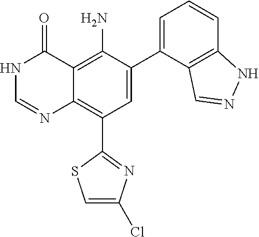 [SMILES: Nc1c(-c2cccc3[nH]ncc23)cc(-c2nc(Cl)cs2)c2nc[nH]c(=O)c12] | 116 |
| 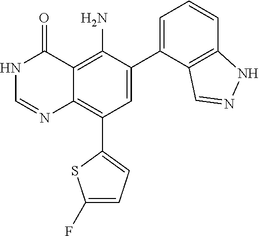 [SMILES: Nc1c(-c2cccc3[nH]ncc23)cc(-c2ccc(F)s2)c2nc[nH]c(=O)c12] | 117 |
| 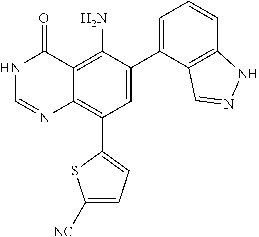 [SMILES: N#Cc1ccc(-c2cc(-c3cccc4[nH]ncc34)c(N)c3c(=O)[nH]cnc23)s1] | 118 |
| 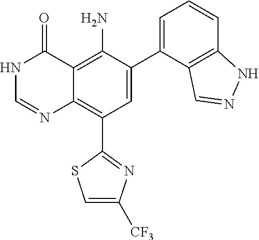 [SMILES: Nc1c(-c2cccc3[nH]ncc23)cc(-c2nc(C(F)(F)F)cs2)c2nc[nH]c(=O)c12] | 119 |
| 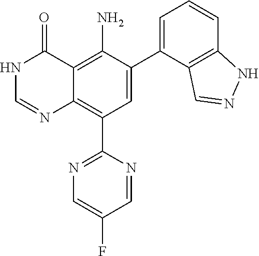 [SMILES: Nc1c(-c2cccc3[nH]ncc23)cc(-c2ncc(F)cn2)c2nc[nH]c(=O)c12] | 120 |
| 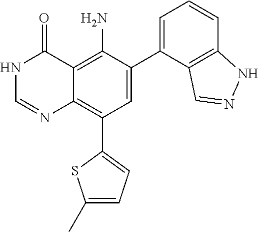 [SMILES: Cc1ccc(-c2cc(-c3cccc4[nH]ncc34)c(N)c3c(=O)[nH]cnc23)s1] | 121 |
| 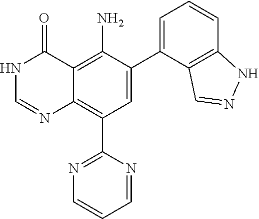 [SMILES: Nc1c(-c2cccc3[nH]ncc23)cc(-c2ncccn2)c2nc[nH]c(=O)c12] | 122 |
| 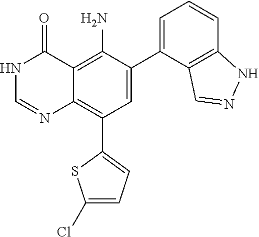 [SMILES: Nc1c(-c2cccc3[nH]ncc23)cc(-c2ccc(Cl)s2)c2nc[nH]c(=O)c12] | 123 |
| 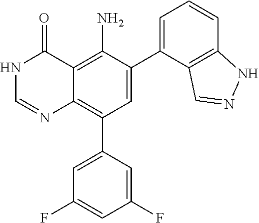 [SMILES: Nc1c(-c2cccc3[nH]ncc23)cc(-c2cc(F)cc(F)c2)c2nc[nH]c(=O)c12] | 124 |
| 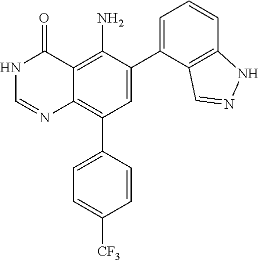 [SMILES: Nc1c(-c2cccc3[nH]ncc23)cc(-c2ccc(C(F)(F)F)cc2)c2nc[nH]c(=O)c12] | 125 |
| 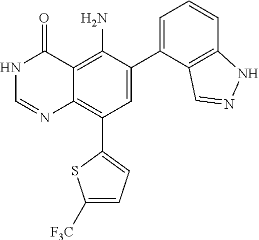 [SMILES: Nc1c(-c2cccc3[nH]ncc23)cc(-c2ccc(C(F)(F)F)s2)c2nc[nH]c(=O)c12] | 126 |
| 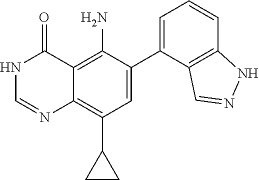 [SMILES: Nc1c(-c2cccc3[nH]ncc23)cc(C2CC2)c2nc[nH]c(=O)c12] | 127 |
| 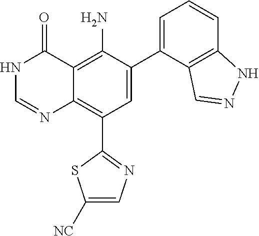 [SMILES: N#Cc1cnc(-c2cc(-c3cccc4[nH]ncc34)c(N)c3c(=O)[nH]cnc23)s1] | 128 |
| 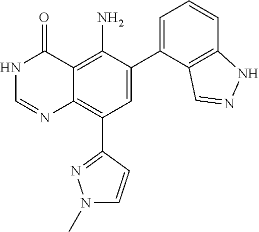 [SMILES: Cn1ccc(-c2cc(-c3cccc4[nH]ncc34)c(N)c3c(=O)[nH]cnc23)n1] | 129 |
| 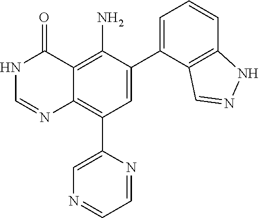 [SMILES: Nc1c(-c2cccc3[nH]ncc23)cc(-c2cnccn2)c2nc[nH]c(=O)c12] | 130 |
| 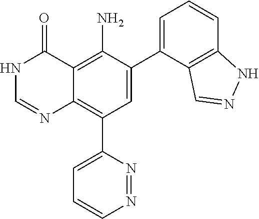 [SMILES: Nc1c(-c2cccc3[nH]ncc23)cc(-c2cccnn2)c2nc[nH]c(=O)c12] | 131 |
| 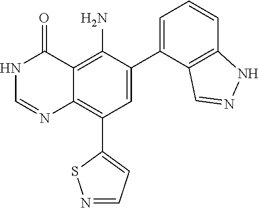 [SMILES: Nc1c(-c2cccc3[nH]ncc23)cc(-c2ccns2)c2nc[nH]c(=O)c12] | 132 |
| 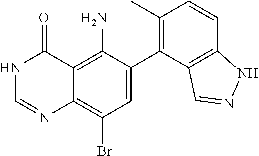 [SMILES: Cc1ccc2[nH]ncc2c1-c1cc(Br)c2nc[nH]c(=O)c2c1N] | 133 |
| 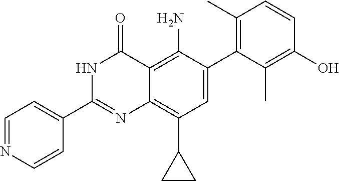 [SMILES: Cc1ccc(O)c(C)c1-c1cc(C2CC2)c2nc(-c3ccncc3)[nH]c(=O)c2c1N] | 134 |
| 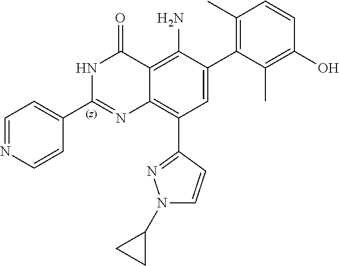 [SMILES: Cc1ccc(O)c(C)c1-c1cc(-c2ccn(C3CC3)n2)c2nc(-c3ccncc3)[nH]c(=O)c2c1N] | 135 |
| 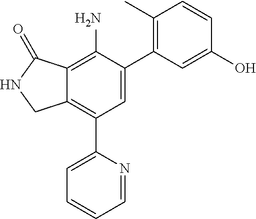 [SMILES: Cc1ccc(O)cc1-c1cc(-c2ccccn2)c2c(c1N)C(=O)NC2] | 136 |
| 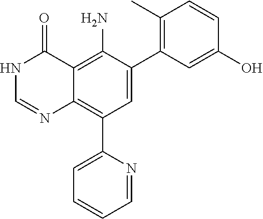 [SMILES: Cc1ccc(O)cc1-c1cc(-c2ccccn2)c2nc[nH]c(=O)c2c1N] | 137 |
| 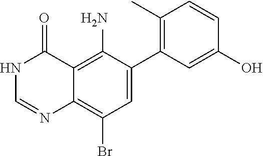 [SMILES: Cc1ccc(O)cc1-c1cc(Br)c2nc[nH]c(=O)c2c1N] | 138 |
| 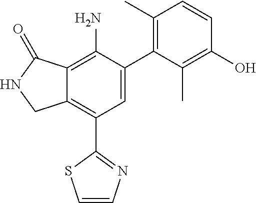 [SMILES: Cc1ccc(O)c(C)c1-c1cc(-c2nccs2)c2c(c1N)C(=O)NC2] | 139 |
| 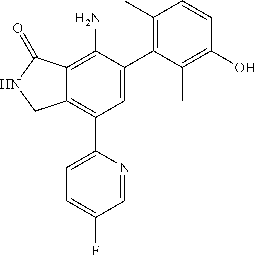 [SMILES: Cc1ccc(O)c(C)c1-c1cc(-c2ccc(F)cn2)c2c(c1N)C(=O)NC2] | 140 |
| 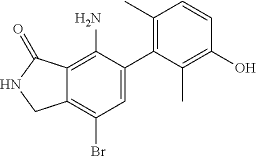 [SMILES: Cc1ccc(O)c(C)c1-c1cc(Br)c2c(c1N)C(=O)NC2] | 141 |
| 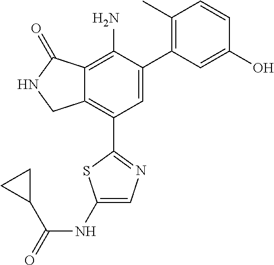 [SMILES: Cc1ccc(O)cc1-c1cc(-c2ncc(NC(=O)C3CC3)s2)c2c(c1N)C(=O)NC2] | 142 |
| 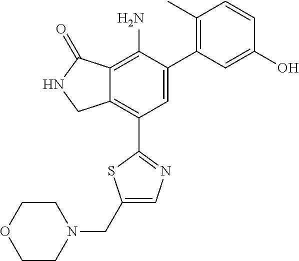 [SMILES: Cc1ccc(O)cc1-c1cc(-c2ncc(CN3CCOCC3)s2)c2c(c1N)C(=O)NC2] | 143 |
| 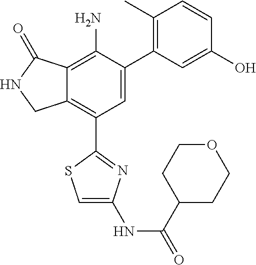 [SMILES: Cc1ccc(O)cc1-c1cc(-c2nc(NC(=O)C3CCOCC3)cs2)c2c(c1N)C(=O)NC2] | 144 |
| 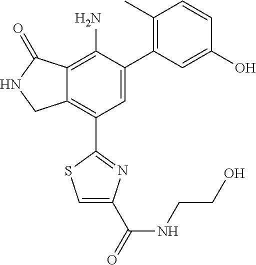 [SMILES: Cc1ccc(O)cc1-c1cc(-c2nc(C(=O)NCCO)cs2)c2c(c1N)C(=O)NC2] | 145 |
| 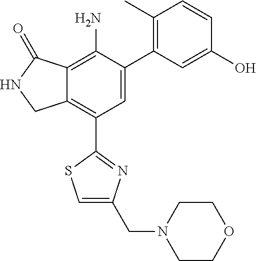 [SMILES: Cc1ccc(O)cc1-c1cc(-c2nc(CN3CCOCC3)cs2)c2c(c1N)C(=O)NC2] | 146 |
| 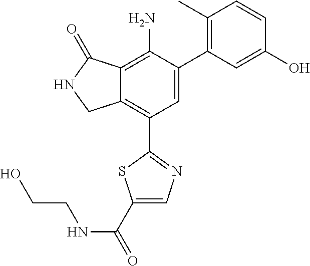 [SMILES: Cc1ccc(O)cc1-c1cc(-c2ncc(C(=O)NCCO)s2)c2c(c1N)C(=O)NC2] | 147 |
| 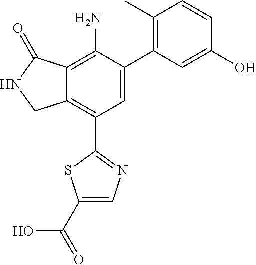 [SMILES: Cc1ccc(O)cc1-c1cc(-c2ncc(C(=O)O)s2)c2c(c1N)C(=O)NC2] | 148 |
| 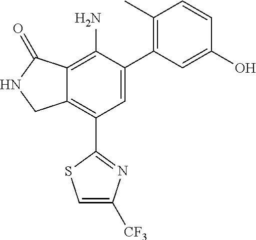 [SMILES: Cc1ccc(O)cc1-c1cc(-c2nc(C(F)(F)F)cs2)c2c(c1N)C(=O)NC2] | 149 |
| 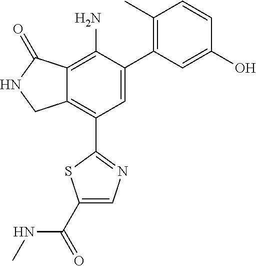 [SMILES: CNC(=O)c1cnc(-c2cc(-c3cc(O)ccc3C)c(N)c3c2CNC3=O)s1] | 150 |
| 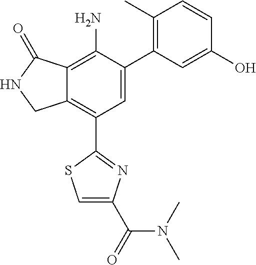 [SMILES: Cc1ccc(O)cc1-c1cc(-c2nc(C(=O)N(C)C)cs2)c2c(c1N)C(=O)NC2] | 151 |
| 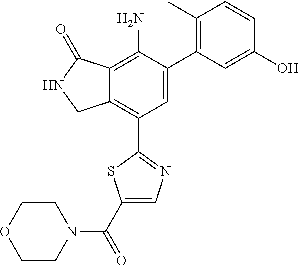 [SMILES: Cc1ccc(O)cc1-c1cc(-c2ncc(C(=O)N3CCOCC3)s2)c2c(c1N)C(=O)NC2] | 152 |
| 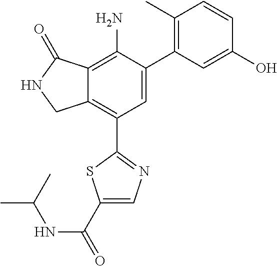 [SMILES: Cc1ccc(O)cc1-c1cc(-c2ncc(C(=O)NC(C)C)s2)c2c(c1N)C(=O)NC2] | 153 |
| 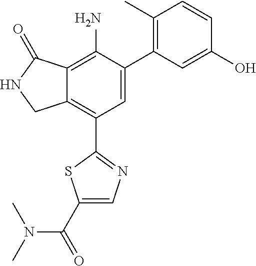 [SMILES: Cc1ccc(O)cc1-c1cc(-c2ncc(C(=O)N(C)C)s2)c2c(c1N)C(=O)NC2] | 154 |
| 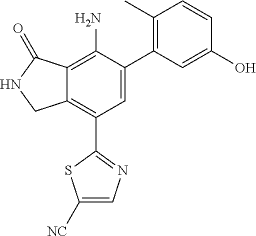 [SMILES: Cc1ccc(O)cc1-c1cc(-c2ncc(C#N)s2)c2c(c1N)C(=O)NC2] | 155 |
| 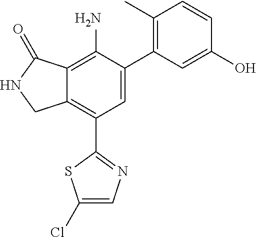 [SMILES: Cc1ccc(O)cc1-c1cc(-c2ncc(Cl)s2)c2c(c1N)C(=O)NC2] | 156 |
| 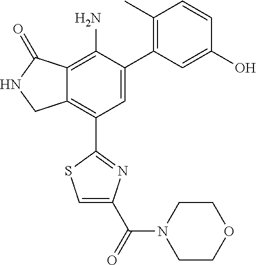 [SMILES: Cc1ccc(O)cc1-c1cc(-c2nc(C(=O)N3CCOCC3)cs2)c2c(c1N)C(=O)NC2] | 157 |
| 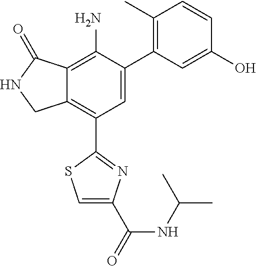 [SMILES: Cc1ccc(O)cc1-c1cc(-c2nc(C(=O)NC(C)C)cs2)c2c(c1N)C(=O)NC2] | 158 |
| 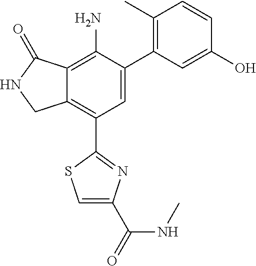 [SMILES: CNC(=O)c1csc(-c2cc(-c3cc(O)ccc3C)c(N)c3c2CNC3=O)n1] | 159 |
| 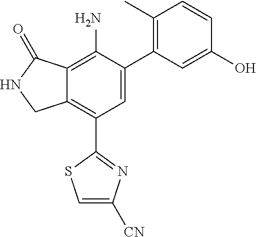 [SMILES: Cc1ccc(O)cc1-c1cc(-c2nc(C#N)cs2)c2c(c1N)C(=O)NC2] | 160 |
| 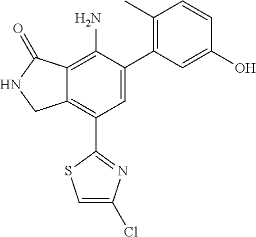 [SMILES: Cc1ccc(O)cc1-c1cc(-c2nc(Cl)cs2)c2c(c1N)C(=O)NC2] | 161 |
| 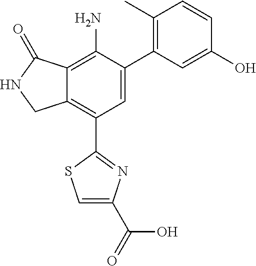 [SMILES: Cc1ccc(O)cc1-c1cc(-c2nc(C(=O)O)cs2)c2c(c1N)C(=O)NC2] | 162 |
| 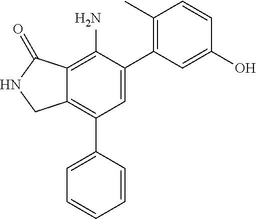 [SMILES: Cc1ccc(O)cc1-c1cc(-c2ccccc2)c2c(c1N)C(=O)NC2] | 163 |
| 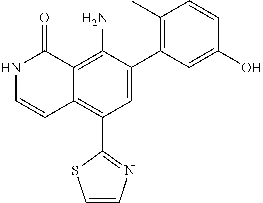 [SMILES: Cc1ccc(O)cc1-c1cc(-c2nccs2)c2cc[nH]c(=O)c2c1N] | 164 |
| 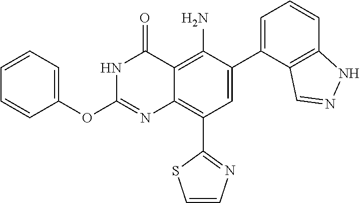 [SMILES: Nc1c(-c2cccc3[nH]ncc23)cc(-c2nccs2)c2nc(Oc3ccccc3)[nH]c(=O)c12] | 165 |

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating Myt1-mediated diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions comprising the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions comprising (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

OpticalIsomers-Diastereomers-GeometricIsomers-Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

Metabolites—Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

Isotopically Enriched Derivatives

The invention includes molecules which have been isotopically enriched at one or more position within the molecule. Thus, compounds enriched for deuterium fall within the scope of the claims.

### Methods of Preparing a Compound of the Invention

Compounds of the present invention may be prepared using reactions and techniques known in the art and those described herein. One of skill in the art will appreciate that methods of preparing compounds of the invention described herein are non-limiting and that steps within the methods may be interchangeable without affecting the structure of the end product.

### Method A

Compounds of the present invention may be prepared as shown in Scheme A and described herein. Ortho amino benzoic acid derivatives such as Intermediate C can be cyclized upon treatment with an acyl chloride and a base. Depending on the nature of the aryl, a protecting group may be required to be in place prior to this reaction. Treatment with ammonia and reduction of the nitro can yield the amino-quinazolinone. Alternatively, the amino-quinazolinone can also be obtained when the nitro reduction is performed prior to the treatment with ammonia. Aryl deprotection if needed can afford compounds of the present invention. Alternatively, when R⁸ is a halogen such as bromo intermediate D, the halogen may be derivatized in a number of different ways at any stage of the synthesis, preferably right after the amino-quinazolinone formation, to afford compounds of the present invention. In the case where the R⁸ group bears an unsaturation, a hydrogenation reaction may be required to give compound of the present invention. In the case where the R⁸ group bear a protecting group, a deprotection step(s) may be required using acid, base, hydrogen and/or fluoride to give compounds of the present invention. Depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

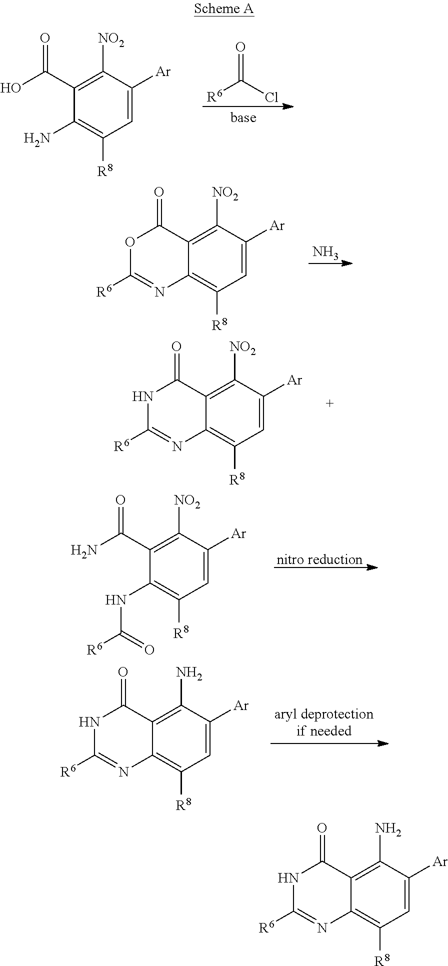

### Method B

Compounds of the present invention may be prepared as shown in Scheme B and described herein. Ortho amino benzoic acid derivatives such as Intermediate C or D can be cyclized upon treatment with an acyl chloride and a base, then treated with a primary amine to afford a bis-amide intermediate. Depending on the nature of the aryl, a protecting group may be required to be in place prior to these reactions. Alternatively, the bis-amide intermediate can be prepared by amide formation from the carboxylic acid and then acylation of the aniline. The bis-amide intermediate can be cyclized to the quinazolinone. Reduction of the nitro can afford the aniline intermediate. R⁶ and R⁸ can be derivatized in a number of ways at different stage of the synthesis, preferably right after the nitro reduction. R^{B3} and the aryl protecting group if needed can be deprotected simultaneously or independently to afford compounds of the present invention. In the case where the R⁶ and/or R⁸ group bears an unsaturation, a hydrogenation reaction may be required to give compound of the present invention. In the case where the R⁶ and/or R⁸ group bear a protecting group, a deprotection step(s) may be required using acid, base, hydrogen and/or fluoride to give compounds of the present invention. Depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

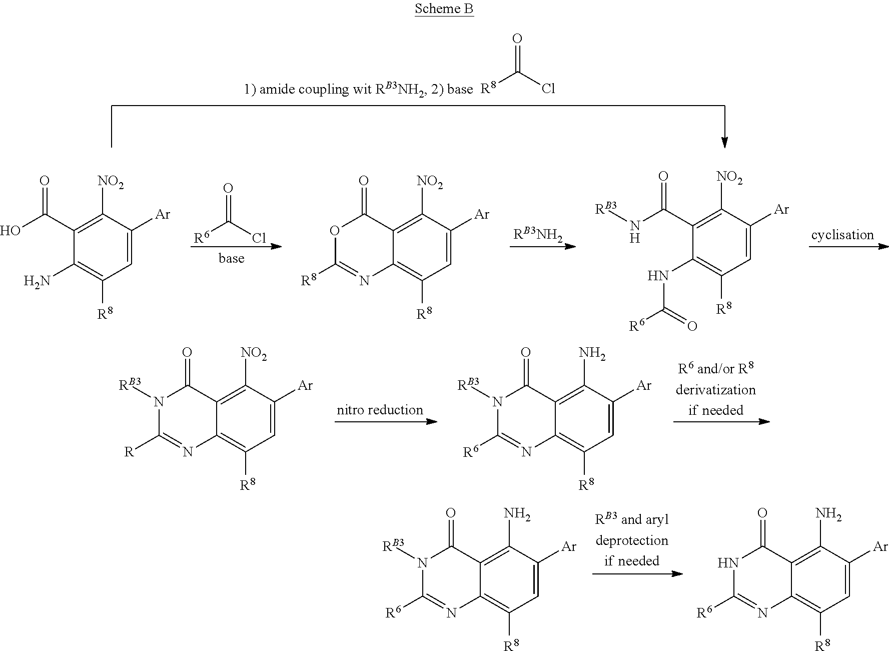

### Method C

Compounds of the present invention may be prepared as shown in Scheme C and described herein. 6-iodoquinazolin-4(3H)-one can be nitrated, and the lactam can be protected with a suitable protecting group such as SEM. The iodo may be substituted by an aryl under metal-mediated conditions. Depending on the nature of the aryl, a protecting group may be required to be in place prior to this reaction. The nitro can be reduced to the aniline and a halogen may then be introduced. The halogen may be carried as such to afford compounds of the present invention after SEM and aryl deprotection if needed. Alternatively, the halogen may be derivatized in a number of different ways prior or after SEM and aryl deprotection if needed. In the case where the R⁸ group bears an unsaturation, a hydrogenation reaction may be required to give compound of the present invention. In the case where the R⁸ group bears a protecting group, a deprotection step(s) may be required using acid, base, hydrogen and/or fluoride to give compounds of the present invention. In the case where R⁸ bears a suitable functional group it can be derivatized by methods known in the art such as SNAR with 2-fluoropyridine or amide coupling reactions. Depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

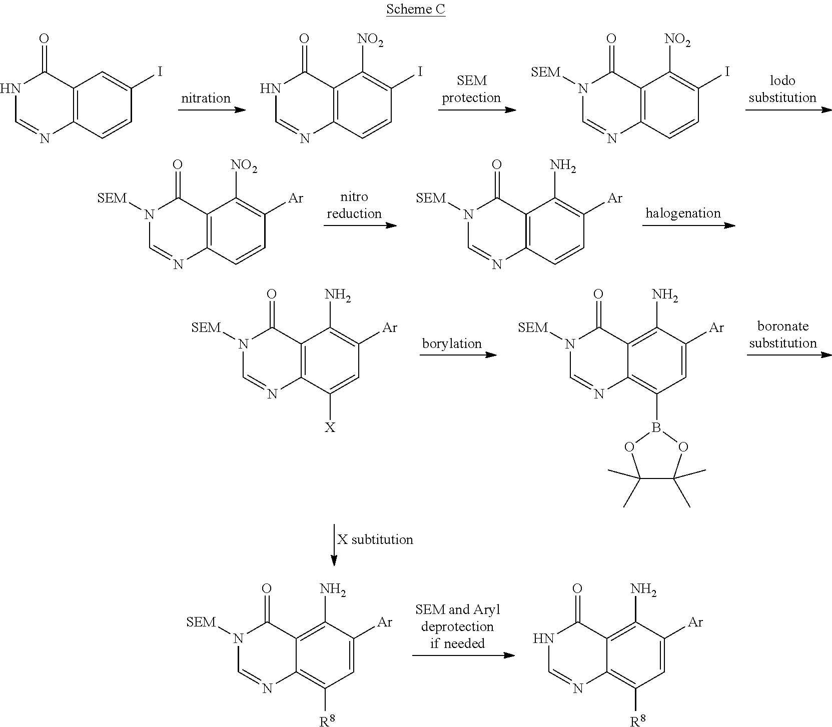

### Method D

Compounds of the present invention may be prepared as shown in Scheme D and described herein. The methyl of methyl 3-bromo-2-methylbenzoate can be brominated and the resulting intermediate can yield a lactam upon treatment with ammonia. Nitration followed by nitro reduction can afford the aniline which can be iodinated. The iodo may be substituted with a suitable aryl. Depending on the nature of the aryl, a protecting group may be required to be in place prior to this reaction. The bromo may be derivatized in a number of different ways prior or after aryl deprotection if needed to afford compounds of the present invention. In the case where the R⁸ group bears an unsaturation, a hydrogenation reaction may be required to give compound of the present invention. In the case where the R⁸ group bears a protecting group, a deprotection step(s) may be required using acid, base, hydrogen and/or fluoride to give compounds of the present invention. In the case where R⁸ group bears a suitable functional group it can be derivatized by methods known in the art such as SNAR with 2-fluoropyridine or amide coupling reactions. Depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

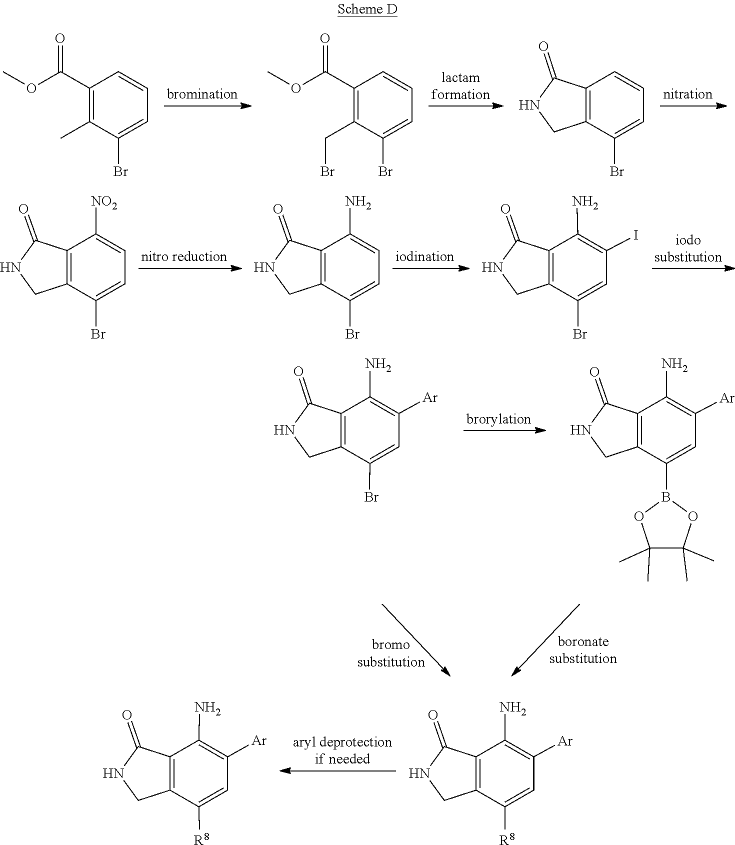

### Method E

Compounds of the present invention may be prepared as shown in Scheme E and described herein. The bromo of 8-bromoisoquinolin-1(2H)-one can be substituted by an amine and the resulting aniline can be brominated, then iodinated. The iodo can be substituted by a suitable aryl. Depending on the nature of the aryl, a protecting group may be required to be in place prior to this reaction. The bromo may be derivatized in a number of different ways prior or after aryl deprotection if needed to afford compounds of the present invention. In the case where the R⁸ group bears an unsaturation, a hydrogenation reaction may be required to give compound of the present invention. In the case where the R⁸ group bears a protecting group, a deprotection step(s) may be required using acid, base, hydrogen and/or fluoride to give compounds of the present invention. In the case where R⁸ group bears a suitable functional group it can be derivatized by methods known in the art. Depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

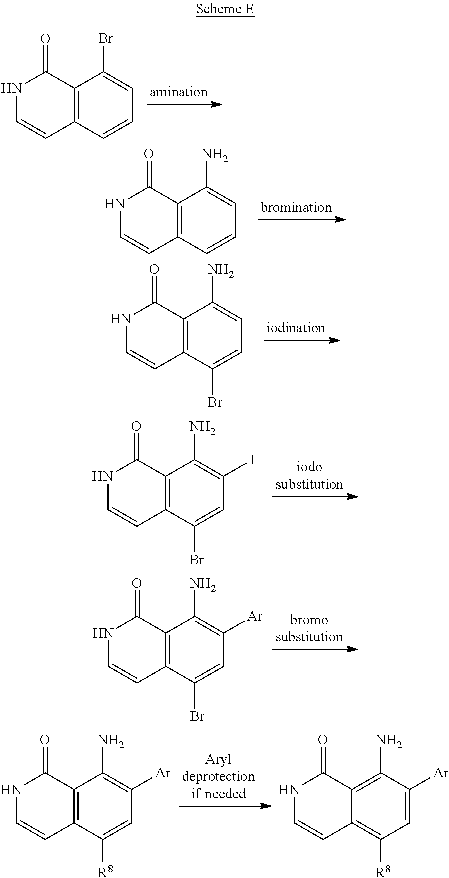

### Method F

Compounds of the present invention may be prepared as shown in Scheme E and described herein. Depending on the nature of the aryl, compounds of the present invention may be obtained as an atropisomeric mixture after final aryl deprotection. In such cases it may be necessary to isolate the atropisomer of interest to give compounds of the present invention. Alternatively, an atropisomerically pure intermediate can be isolated and may be derivatized further to give compounds of the present invention.

Compounds of the present invention may be prepared as shown in Scheme F and described herein. After suitable aryl deprotection, depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

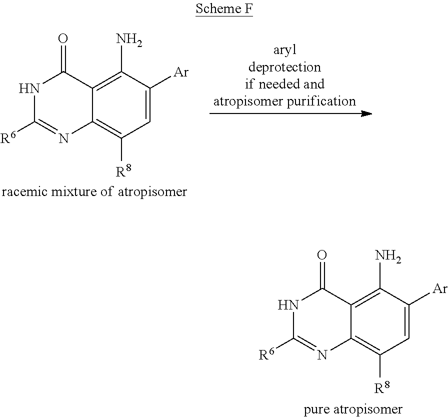

### Method G

Compounds of the present invention may be prepared as shown in Scheme G and described herein. 2-amino-6-nitrobenzoic acid can be cyclized to 5-nitroquinazoline-2,4-diol that can be chlorinated to 2,4-dichloro-5-nitroquinazoline. One chloro can be substituted by a nucleophile such as methoxide. The nitro can be reduced to the aniline that can be brominated then iodinated. The iodo can be substituted by a suitable aryl. Depending on the nature of the aryl, a protecting group may be required to be in place prior to this reaction. The remaining chloro can be substituted by a nucleophile such as phenoxide and the bromo may be derivatized in a number of different ways prior or after aryl deprotection if needed to afford compounds of the present invention. In the case where the R⁸ group bears an unsaturation, a hydrogenation reaction may be required to give compound of the present invention. In the case where the R⁸ group bear a protecting group, a deprotection step(s) may be required using acid, base, hydrogen and/or fluoride to give compounds of the present invention. In the case where R⁸ group bears a suitable functional group it can be derivatized by methods known in the art. Depending on the nature of the aryl, an atropisomeric mixture may be obtain. In such cases it may be necessary to isolate the atropisomer of interest to give compounds of the present invention. Alternatively, an atropisomerically pure intermediate can be isolated and may be derivatized further to give compounds of the present invention.

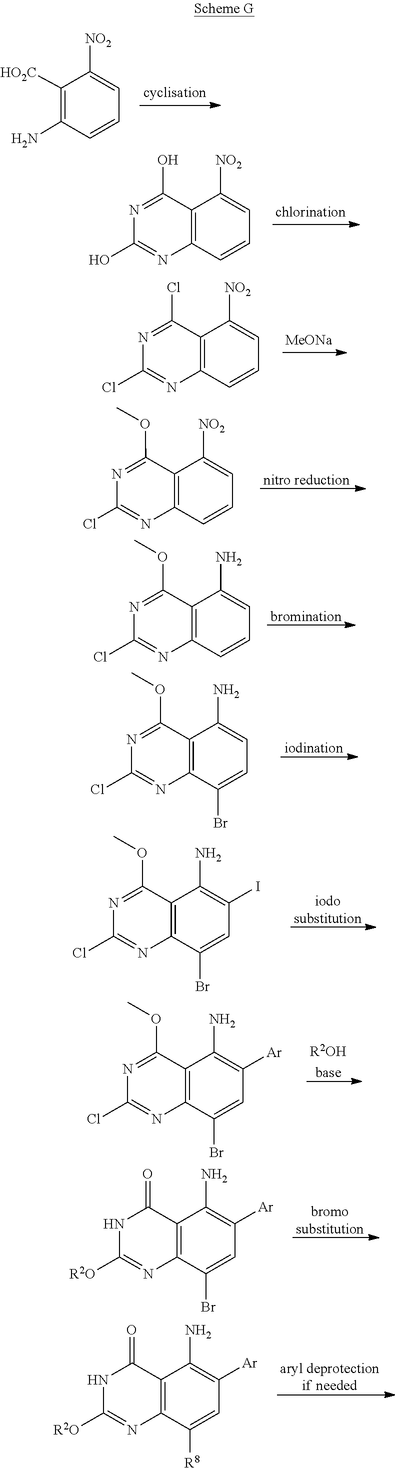

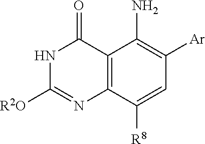

### Methods of Treatment

Compounds of the invention may be used for the treatment of a disease or condition (e.g., a cancer overexpressing CCNE1 or having an inactivating mutation in the FBXW7 gene) which depend on the activity of Myt1 (Gene name PKMYT1).

The disease or condition may have the symptom of cell hyperproliferation. For example, the disease or condition may be a cancer (e.g., a cancer overexpressing CCNE1 or having an inactivating mutation in the FBXW7 gene).

Cancers which have a high incidence of CCNE1 overexpression include e.g., uterine cancer, ovarian cancer, breast cancer, stomach cancer, esophageal cancer, lung cancer, and endometrial cancer.

Cancers which have a deficiency in FBXW7 include, e.g., uterine cancer, colorectal cancer, breast cancer, lung cancer, and esophageal cancer.

A compound of the invention may be administered by a route selected from the group consisting of oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, intratumoral, and topical administration.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers including excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted over time according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In some embodiments, the compounds are administered to the patient over a time period that is, for example, 4-11 months or 1-30 years. In some embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

### Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a disease or condition. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) Drug Injection: a liquid preparation that is a drug substance (e.g., a compound of the invention), or a solution thereof;
(2) Drug for Injection: the drug substance (e.g., a compound of the invention) as a dry solid that will be combined with the appropriate sterile vehicle for parenteral administration as a drug injection;
(3) Drug Injectable Emulsion: a liquid preparation of the drug substance (e.g., a compound of the invention) that is dissolved or dispersed in a suitable emulsion medium;
(4) Drug Injectable Suspension: a liquid preparation of the drug substance (e.g., a compound of the invention) suspended in a suitable liquid medium; and
(5) Drug for Injectable Suspension: the drug substance (e.g., a compound of the invention) as a dry solid that will be combined with the appropriate sterile vehicle for parenteral administration as a drug injectable suspension.

Formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Combinations

Compounds of the present invention may be administered to a subject in combination with one or more additional agents, e.g.:
(a) a cytotoxic agent;
(b) an antimetabolite;
(c) an alkylating agent;
(d) an anthracycline;
(e) an antibiotic;
(f) an anti-mitotic agent;
(g) a hormone therapy;
(h) a signal transduction inhibitor;
(i) a gene expression modulator;
(j) an apoptosis inducer;
(k) an angiogenesis inhibitor;
(l) an immunotherapy agent;
(m) a DNA damage repair inhibitor;
or
a combination thereof.

The cytotoxic agent may be, e.g., actinomycin-D, alemtuzumab, alitretinoin, allopurinol, altretamine, amifostine, amphotericin, amsacrine, arsenic trioxide, asparaginase, azacitidine, azathioprine, Bacille Calmette-Guérin (BCG), bendamustine, bexarotene, bevacuzimab, bleomycin, bortezomib, busulphan, capecitabine, carboplatin, carfilzomib, carmustine, cetuximab, cisplatin, chlorambucil, cladribine, clofarabine, colchicine, crisantaspase, cyclophosphamide, cyclosporine, cytarabine, cytochalasin B, dacarbazine, dactinomycin, darbepoetin alfa, dasatinib, daunorubicin, 1-dehydrotestosterone, denileukin, dexamethasone, dexrazoxane, dihydroxy anthracin dione, disulfiram, docetaxel, doxorubicin, emetine, epirubicin, erlotinib, epigallocatechin gallate, epoetin alfa, estramustine, ethidium bromide, etoposide, everolimus, filgrastim, finasunate, floxuridine, fludarabine, flurouracil (5-FU), fulvestrant, ganciclovir, geldanamycin, gemcitabine, glucocorticoids, gramicidin D, histrelin acetate, hydroxyurea, ibritumomab, idarubicin, ifosfamide, imatinib, irinotecan, interferons, interferon alfa-2a, interferon alfa-2b, ixabepilone, lactate dehydrogenase A (LDH-A), lenalidomide, letrozole, leucovorin, levamisole, lidocaine, lomustine, mechlorethamine, melphalan, 6-mercaptopurine, mesna, methotrexate, methoxsalen, metoprine, metronidazole, mithramycin, mitomycin-C, mitoxantrone, nandrolone, nelarabine, nilotinib, nofetumomab, oprelvekin, oxaliplatin, paclitaxel, pemetrexed, pentostatin, palifermin, pamidronate, pegademase, pegaspargase, pegfilgrastim, pemetrexed disodium, plicamycin, porfimer sodium, procaine, procarbazine, propranolol, puromycin, quinacrine, radicicol, radioactive isotopes, raltitrexed, rapamycin, rasburicase, salinosporamide A, sargramostim, sunitinib, temozolomide, teniposide, tetracaine, 6-thioguanine, thiotepa, topotecan, toremifene, trastuzumab, treosulfan, tretinoin, valrubicin, vinblastine, vincristine, vindesine, vinorelbine, zoledronate, or a combination thereof.

The antimetabolites may be, e.g., methotrexate, 6-mercaptopurine, 6-thioguanine, cytarabine, 5-fluorouracil decarbazine, cladribine, pemetrexed, gemcitabine, capecitabine, hydroxyurea, mercaptopurine, fludarabine, pralatrexate, clofarabine, cytarabine, decitabine, floxuridine, nelarabine, trimetrexate, thioguanine, pentostatin, or a combination thereof.

The alkylating agent may be, e.g., mechlorethamine, thiotepa, chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, altretamine, cyclophosphamide, ifosfamide, hexamethylmelamine, altretamine, procarbazine, dacarbazine, temozolomide, streptozocin, carboplatin, cisplatin, oxaliplatin, uramustine, bendamustine, trabectedin, semustine, or a combination thereof.

The anthracycline may be, e.g., daunorubicin, doxorubicin, aclarubicin, aldoxorubicin, amrubicin, annamycin, carubicin, epirubicin, idarubicin, mitoxantrone, valrubicin, or a combination thereof.

The antibiotic may be, e.g., dactinomycin, bleomycin, mithramycin, anthramycin (AMC), ampicillin, bacampicillin, carbenicillin, cloxacillin, dicloxacillin, flucloxacillin, mezlocillin, nafcillin, oxacillin, piperacillin, pivampicillin, pivmecillinam, ticarcillin, aztreonam, imipenem, doripenem, ertapenem, meropenem, cephalosporins, clarithromycin, dirithromycin, roxithromycin, telithromycin, lincomycin, pristinamycin, quinupristin, amikacin, gentamicin, kanamycin, neomycin, netilmicin, paromomycin, tobramycin, streptomycin, sulfamethizole, sulfamethoxazole, sulfisoxazole, demeclocycline, minocycline, oxytetracycline, tetracycline, penicillin, amoxicillin, cephalexin, erythromycin, clarithromycin, azithromycin, ciprofloxacin, levofloxacin, ofloxacin, doxycycline, clindamycin, metronidazole, tigecycline, chloramphenicol, metronidazole, tinidazole, nitrofurantoin, vancomycin, teicoplanin, telavancin, linezolid, cycloserine, rifamycins, polymyxin B, bacitracin, viomycin, capreomycin, quinolones, daunorubicin, doxorubicin, 4′-deoxydoxorubicin, epirubicin, idarubicin, plicamycin, mitomycin-c, mitoxantrone, or a combination thereof.

The anti-mitotic agent may be, e.g., vincristine, vinblastine, vinorelbine, docetaxel, estramustine, ixabepilone, paclitaxel, maytansinoid, a dolastatin, a cryptophycin, or a combination thereof.

The signal transduction inhibitor may be, e.g., imatinib, trastuzumab, erlotinib, sorafenib, sunitinib, temsirolimus, vemurafenib, lapatinib, bortezomib, cetuximab panitumumab, matuzumab, gefitinib, STI 571, rapamycin, flavopiridol, imatinib mesylate, vatalanib, semaxinib, motesanib, axitinib, afatinib, bosutinib, crizotinib, cabozantinib, dasatinib, entrectinib, pazopanib, lapatinib, vandetanib, or a combination thereof.

The gene expression modulator may be, e.g., a siRNA, a shRNA, an antisense oligonucleotide, an HDAC inhibitor, or a combination thereof. An HDAC inhibitor may be, e.g., trichostatin A, trapoxin B, valproic acid, vorinostat, belinostat, LAQ824, panobinostat, entinostat, tacedinaline, mocetionstat, givinostat, resminostat, abexinostat, quisinostat, rocilinostat, practinostat, CHR-3996, butyric acid, phenylbutyric acid, 4SC202, romidepsin, sirtinol, cambinol, EX-527, nicotinamide, or a combination thereof. An antisense oligonucleotide may be, e.g., custirsen, apatorsen, AZD9150, trabadersen, EZN-2968, LErafAON-ETU, or a combination thereof. An siRNA may be, e.g., ALN-VSP, CALAA-01, Atu-027, SPC2996, or a combination thereof.

The hormone therapy may be, e.g., a luteinizing hormone-releasing hormone (LHRH) antagonist. The hormone therapy may be, e.g., firmagon, leuproline, goserelin, buserelin, flutamide, bicalutadmide, ketoconazole, aminoglutethimide, prednisone, hydroxyl-progesterone caproate, medroxy-progesterone acetate, megestrol acetate, diethylstil-bestrol, ethinyl estradiol, tamoxifen, testosterone propionate, fluoxymesterone, flutamide, raloxifene, droloxifene, iodoxyfene, 4-hydroxytamoxifen, trioxifene, keoxifene, LY117018, onapristone, toremifine citrate, megestrol acetate, exemestane, fadrozole, vorozole, letrozole, anastrozole, nilutamide, tripterelin, histerelin, arbiraterone, medroxyprogesterone acetate, diethylstilbestrol, premarin, fluoxymesterone, tretinoin, fenretinide, troxacitabine, or a combination thereof.

The apoptosis inducers may be, e.g., a recombinant human TNF-related apoptosis-inducing ligand (TRAIL), camptothecin, bortezomib, etoposide, tamoxifen, or a combination thereof.

The angiogenesis inhibitors may be, e.g., sorafenib, sunitinib, pazopanib, everolimus or a combination thereof.

The immunotherapy agent may be, e.g., a monoclonal antibody, cancer vaccine (e.g., a dendritic cell (DC) vaccine), oncolytic virus, cytokine, adoptive T cell therapy, Bacille Calmette-Guérin (BCG), GM-CSF, thalidomide, lenalidomide, pomalidomide, imiquimod, or a combination thereof. The monoclonal antibody may be, e.g., anti-CTLA4, anti-PD1, anti-PD-L¹, anti-LAG3, anti-KIR, or a combination thereof. The monoclonal antibody may be, e.g., alemtuzumab, trastuzumab, ibritumomab tiuxetan, brentuximab vedotin, trastuzumab, ado-trastuzumab emtansine, blinatumomab, bevacizumab, cetuximab, pertuzumab, panitumumab, ramucirumab, obinutuzumab, ofatumumab, rituximab, pertuzumab, tositumomab, gemtuzumab ozogamicin, tositumomab, or a combination thereof. The cancer vaccine may be, e.g., Sipuleucel-T, BioVaxID, NeuVax, DCVax, SuVaxM, CIMAvax®, Provenge,®, hsp110 chaperone complex vaccine, CDX-1401, MIS416, CDX-110, GVAX Pancreas, HyperAcute™ Pancreas, GTOP-99 (MyVax®), or Imprime PGG®. The oncolytic virus may be, e.g., talimogene laherparepvec. The cytokine may be, e.g., IL-2, IFNα, or a combination thereof. The adoptive T cell therapy may be, e.g., tisagenlecleucel, axicabtagene ciloleucel, or a combination thereof.

The DNA damage repair inhibitor may be, e.g., a PARP inhibitor, a cell checkpoint kinase inhibitor, or a combination thereof. The PARP inhibitor may be, e.g., olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338 or a combination thereof. The cell checkpoint kinase inhibitor may be, e.g., MK-1775 or AZD1775, AZD7762, LY2606368, PF-0477736, AZD0156, GDC-0575, ARRY-575, CCT245737, PNT-737 or a combination thereof.

### EXAMPLES

The following examples were meant to illustrate the invention. They were not meant to limit the invention in any way.

Reactions were typically performed at room temperature (rt or RD under a nitrogen atmosphere using dry solvents (Sure/Seal™) if not described otherwise in the Examples below. Reactions were monitored by TLC or by injection of a small aliquot on a Waters Acquity-H UPLC® Class system using an Acquity® UPLC HSS C18 2.1×30 mm column eluting with a gradient (1.86 min) of acetonitrile (15% to 98%) in water (both containing 0.1% formic acid). Purifications by preparative HPLC were performed on a Teledyne Isco Combi Flash® EZ Prep system using either Phenomenex Gemini® 5 μm NX-C18 110 Å 150×21.2 mm column at a flow of 40 mL/min over 12 min (<100 mg or multiple injections of <100 mg) or HP C18 RediSep® Rf gold column (>100 mg) eluting with an appropriate gradient of acetonitrile in water (both containing 0.1% formic acid) unless otherwise specified. The gradient was selected based on the retention time observed by reaction monitoring on the Waters Acquity-H UPLC® Class system (see above). Fractions containing the desired compounds were combined and finally lyophilized. Purifications by silica gel chromatography were performed on a Teledyne Isco Combi Flash® Rf system using RediSep® Rf silica gel columns of appropriate sizes. Purity of final Compounds was assessed by injection of a small aliquot on a Waters Acquity-H UPLC® Class system using an Acquity® UPLC BEH C18 2.1×50 mm column eluting with a gradient (7 min) of acetonitrile (2% to 98%) in water (both containing 0.1% formic acid).

### Example 1. Preparation of Compounds

### Intermediates

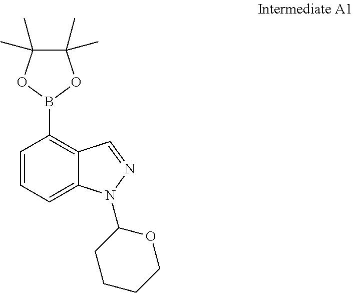 [SMILES: CC1(C)OB(c2cccc3c2cnn3C2CCCCO2)OC1(C)C]

Intermediate A1 can be prepared as described in WO 2014/169167.

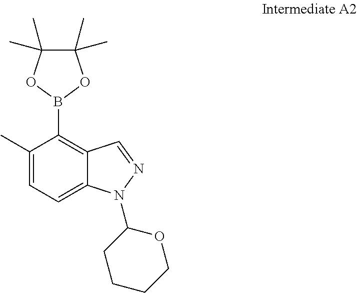 [SMILES: Cc1ccc2c(cnn2C2CCCCO2)c1B1OC(C)(C)C(C)(C)O1]

Intermediate A2 can be prepared as described in CN 111484477

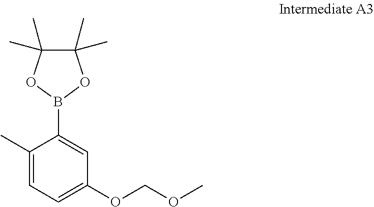

Intermediate A3 can be prepared as described in Craig, et al., Chemical Communications, 2020, 56:9505-9508.

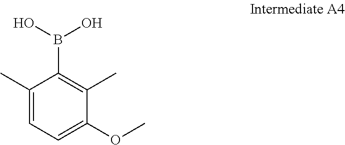

Intermediate A4 can be prepared as described in WO 2004/43925.

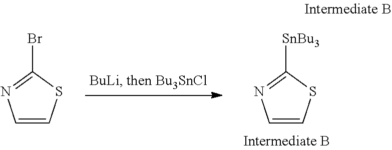

Intermediate B can be prepared as described in JP 2016/56276

Intermediates C and D

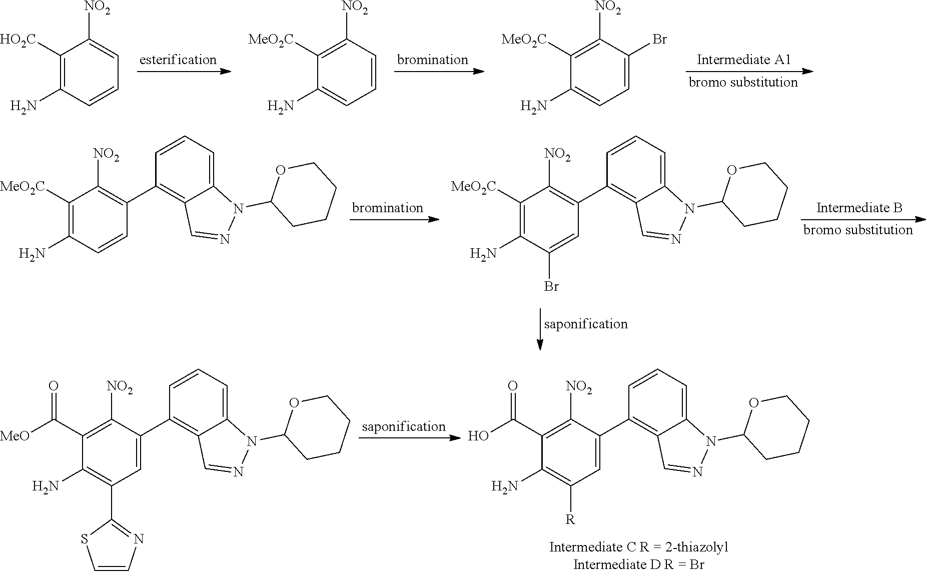

A three-neck round bottom flask (500 mL) under nitrogen equipped with a mechanical stirrer and a heating mantel controlled by a J-KEM 260 was loaded with 2-amino-6-nitrobenzoic acid (25.0 g, 137.36 mmol) and DMF (150 mL). The reaction mixture was stirred until complete dissolution of 2-amino-6-nitrobenzoic acid. Potassium carbonate (18.98 g, 137.36 mmol) was then added in portions. After the addition of potassium carbonate, dimethyl sulfate (15.6 mL, 164.84 mmol) was added dropwise via a syringe. The resulting mixture was heated at 60° C. for 18 h. Upon cooling the reaction mixture to rt, the aqueous saturated NH₄Cl (6.5 mL) was added followed by dropwise addition of water (175 mL). The solid slowly precipitated, and the stirring was kept for 2 h. Additional water (100 mL) was added, and the resulting suspension was stirred for 1 h. The solid was collected by filtration and washed with water (250 mL) and heptane (100 mL). The collected solid was dried under high vacuum for 18 h to afford methyl 2-amino-6-nitrobenzoate (21.7 g) as an orange yellow solid. ¹H-NMR (CHCl₃-d, 400 MHz): δ 7.25-7.29 (m, 1H), 7.02 (dd, J=7.8, 1.1 Hz, 1H), 6.87 (dd, J=8.4, 1.1 Hz, 1H), 5.30 (s, 2H), 3.83 (s, 3H). LCMS (+ESI): m/z=197.1 [M+1]⁺.

A round bottom flask (1 L) was loaded with methyl 2-amino-6-nitrobenzoate (21.7 g, 110.6 mmol), CaCO₃ (44.28 g, 442.4 mmol), MeOH (87 mL) and DCM (217 mL). The resulting mixture was degassed by bubbling nitrogen for 5 minutes and cooled to 0° C. A solution of benzyltrimethylammonium tribromide (47.44 g, 121.66 mmol) in degassed DCM (108 mL) was slowly added to the reaction mixture over 45 minutes. After the addition of benzyltrimethylammonium tribromide, the reaction mixture was stirred at 0° C. for 1 h, then filtered on a Buchner to remove the inorganic salts. The filtrate was concentrated in vacuo and the residue was dissolved in EtOAc and the resulting solution was quenched with water. The layers were partitioned, and the aqueous layer was extracted with EtOAc. The combined organic was washed with water (2×) and brine, dried over anhydrous MgSO₄, filtered and concentrated. Heptane (200 mL) was slowly added to the residue and the resulting suspension was stirred for 1 h. The solids were collected by filtration and dried in the vacuum oven to afford methyl 6-amino-3-bromo-2-nitrobenzoate (27 g) as an orange powder. ¹H-NMR (CHCl₃-d, 400 MHz): δ 7.43 (d, J=9.0 Hz, 1H), 6.68 (d, J=9.0 Hz, 1H), 5.91 (s, 2H), 3.83 (s, 3H). LCMS (+ESI): m/z=275.0/277.0 [M+1]⁺.

A solution of methyl 6-amino-3-bromo-2-nitrobenzoate (10 g, 36.36 mmol), 1-(tetrahydro-2H-pyran-2-yl)-4-(4,4,5,5-tetramethyl-1,3,2-dioxaborolan-2-yl)-1H-indazole (intermediate A1, 13.7 g, 41.74 mmol), PdCl₂(dppf) (1.3 g, 1.78 mmol), NaHCO₃ (2 M in water, 54.5 mL, 92 mmol) and dioxane (100 mL) were degassed (3 cycles of vacuum/nitrogen atmosphere). The mixture was heated at 110° C. for 2 h. Upon cooling to rt, the mixture was diluted with water (300 mL) and EtOAc (400 mL), filtered through celite. The filtrate was partitioned, and the aqueous layer was extracted with EtOAc (300 mL). The combined organic layers were washed with water and brine, dried over anhydrous MgSO₄, filtered, and concentrated in vacuo. The residue was adsorbed on silica gel for purification by ISCO CombiFlash (2×120 g Gold SiO₂) eluting with 20-90% EtOAc/hexanes over 20 min. The desired fractions were combined and concentrated in vacuo to afford methyl 6-amino-2-nitro-3-(1-(tetrahydro-2H-pyran-2-yl)-1H-indazol-4-yl)benzoate (6.4 g) as a yellow solid. LCMS (+ESI): m/z=397.1 [M+1]⁺.

To a solution of methyl 6-amino-2-nitro-3-(1-(tetrahydro-2H-pyran-2-yl)-1H-indazol-4-yl)benzoate (6.4 g, 14.53 mmol) in DCM (64 mL) and MeOH (64 mL) were added benzyltrimethylammonium tribromide (6.2 g, 15.9 mmol) and CaCO₃ (1.7 g, 17.0 mmol). The reaction was stirred at rt for 18 h. The reaction mixture was filtered on Buchner to remove inorganic salts and the solids were washed with DCM. The filtrate was quenched with 10% sodium thiosulfate and the volatiles were removed in vacuo. The resulting mixture was extracted with EtOAc (200 mL) and the aqueous layer was extracted with EtOAc (150 mL). The combined organic layers were washed with brine, dried over anhydrous MgSO₄, filtered and concentrated to dryness. The residue was adsorbed on silica gel for purification by ISCO CombiFlash (120 g Gold SiO₂ column) eluting with 20-60% EtOAc/hexanes to provide methyl 2-amino-3-bromo-6-nitro-5-(1-tetrahydropyran-2-ylindazol-4-yl)benzoate (4.6 g) as a yellow solid. LCMS (+ESI): m/z=475.0/477.0 [M+1]⁺.

A mixture of methyl 2-amino-3-bromo-6-nitro-5-(1-tetrahydropyran-2-ylindazol-4-yl)benzoate (1.72 g, 3.62 mmol), LiCl (314 mg, 7.24 mmol), CuI (138 mg, 0.724 mmol), 2-(tributylstannyl)thiazole (Intermediate B, 2.71 g, 7.24 mmol), PdCl₂(dppf). DCM (148 mg, 0.181 mmol) in DMF (19.26 mL) was degassed (3 cycles of vacuum/nitrogen atmosphere). The resulting mixture was heated at 65° C. for 1 h. Upon cooling to rt, the reaction mixture was quenched with water (20 mL) and EtOAc (100 mL). The resulting mixture was filtered through celite and washed with EtOAc. The layers were partitioned. The organic layer was washed with water (4×) and brine, dried over anhydrous MgSO₄, filtered and concentrated to dryness in vacuo. The residue was adsorbed on silica gel for purification by ISCO CombiFlash (80 g Gold SiO₂ column) eluting with 0-30% EtOAc/hexanes. The desired fractions were combined and concentrated to a slurry, azeotroped with heptane (2×) and concentrated to a slurry. The solids were collected by filtration and dried in the vacuum oven at 40° C. to provide methyl 2-amino-6-nitro-5-(1-(tetrahydro-2H-pyran-2-yl)-1H-indazol-4-yl)-3-(thiazol-2-yl)benzoate (1.44 g) as a yellow powder. ¹H-NMR (CDCl₃, 400 MHz): δ 8.61 (s, 2H), 7.88-7.89 (m, 3H), 7.62 (d, J=8.5 Hz, 1H), 7.40 (t, J=7.8 Hz, 1H), 7.35 (d, J=3.4 Hz, 1H), 7.07 (d, J=7.1 Hz, 1H), 5.76 (dd, J=9.5, 2.5 Hz, 1H), 4.07 (d, J=11.7 Hz, 1H), 3.85 (s, 3H), 3.78 (t, J=10.4 Hz, 1H), 2.61 (d, J=12.0 Hz, 1H), 2.16 (br d, J=14.6 Hz, 3H), 1.73 (d, J=41.3 Hz, 3H).

A round bottom flask (50 mL) under nitrogen atmosphere was charged with methyl 2-amino-6-nitro-5-(1-(tetrahydro-2H-pyran-2-yl)-1H-indazol-4-yl)-3-(thiazol-2-yl)benzoate (1.05 g, 2.19 mmol) and MeOH (14.7 mL). The reaction mixture was cooled at 0° C. and a solution of LiOH (1 N in H₂O, 5.5 mL, 5.5 mmol) was added dropwise over 5 minutes. After the addition of LiOH, the reaction mixture was stirred at rt for 3 h. THF (15 mL) was then added and the reaction mixture was allowed to stir at rt for 16 h. The organics solvents were removed in vacuo and the suspension in water was cooled at 0° C. The pH of the suspension was adjusted to 4-5 by dropwise addition of 1 N HCl (˜3.6 mL). The suspension was dissolved in Me-THF and NaCl was added to saturate the aqueous layer. The layers were partitioned, and the aqueous layer was extracted with Me-THF (2×). The combined organics were concentrated to dryness. The residue was suspended in DCM. The solids were collected by filtration and dried under high vacuum to afford 2-amino-6-nitro-5-(1-(tetrahydro-2H-pyran-2-yl)-1H-indazol-4-yl)-3-(thiazol-2-yl)benzoic acid intermediate C (0.95 g). LCMS (+ESI): m/z=466.1 [M+1]⁺.

To a solution of methyl 2-amino-3-bromo-6-nitro-5-(1-(tetrahydro-2H-pyran-2-yl)-1H-indazol-4-yl)benzoate (2.4 g, 5.05 mmol) in THF (6 mL), MeOH (2 mL) and water (2 mL) was added LiOH (2.42 g, 101 mmol). The reaction mixture was stirred at rt for 72 h. The pH of the reaction was adjusted to 3-4 by addition of HCl (2 M). Solids were formed, collected by filtration and dried under high vacuum to afford 2-amino-3-bromo-6-nitro-5-(1-(tetrahydro-2H-pyran-2-yl)-1H-indazol-4-yl)benzoic acid, intermediate D, (2.3 g) which was used as such in the subsequent step without further purification. LCMS (+ESI): m/z=460.9/462.8 [M+1]⁺.

### Compounds Synthesized by Method A

### Compound 1

Benzoyl chloride (49.7 mg, 0.258 mmol) was added to a solution of 2-amino-6-nitro-5-(1-(tetrahydro-2H-pyran-2-yl)-1H-indazol-4-yl)-3-(thiazol-2-yl)benzoic acid Intermediate C (100 mg, 0.216 mmol), DMAP (2.6 mg, 0.022 mmol) and triethylamine (1194, 0.861 mmol) in DMF (3 mL). The reaction mixture was stirred at rt for 2 days. The reaction mixture was then diluted with water. Solids were formed, collected by filtration and washed with water. The collected solids were dried under high vacuum to afford 5-nitro-2-phenyl-6-(1-(tetrahydro-2H-pyran-2-yl)-1H-indazol-4-yl)-8-(thiazol-2-yl)-4H-benzo[d][1,3]oxazin-4-one (118 mg) which was used as such in the subsequent step without further purification. LCMS (+ESI): m/z=552.1 [M+1]⁺.

A sealable reaction tube was loaded with 5-nitro-2-phenyl-6-(1-(tetrahydro-2H-pyran-2-yl)-1H-indazol-4-yl)-8-(thiazol-2-yl)-4H-benzo[d][1,3]oxazin-4-one (118 mg, 0.214 mmol) and MeOH (2 mL). The reaction mixture was cooled at −78° C. and NH₃ gas was bubbled into the reaction mixture for 3 minutes. The reaction tube was then sealed and heated at 120° C. for 16 h. Upon cooling to rt, the reaction mixture was diluted with water and was extracted with EtOAc (2×). The combined organic layers were dried over anhydrous MgSO₄, filtered and concentrated to dryness in vacuo. The residue was purified by silica gel chromatography eluting with 0-90% EtOAc/hexanes over 20 min to afford 5-nitro-2-phenyl-6-(1-(tetrahydro-2H-pyran-2-yl)-1H-indazol-4-yl)-8-(thiazol-2-yl)quinazolin-4(3H)-one (13 mg). LCMS (+ESI): m/z=551.9 [M+1]⁺.

Platinum oxide (10.3 mg, 0.045 mnmol) was added to a solution of 5-nitro-2-phenyl-6-(1-(tetrahydro-2H-pyran-2-yl)-1H-indazol-4-yl)-8-(thiazol-2-yl)quinazolin-4(3H)-one (50 mg, 0.091 mmol) in AcOH (2 mL) at rt. The reaction mixture was stirred under hydrogen atmosphere (in balloon) for 18 h. The reaction mixture was filtered using celite and washed with DCM and EtOAc. The filtrate was concentrated to dryness in vacuo to afford 15 mg of a mixture of 5-amino-2-phenyl-6-(1-(tetrahydro-2H-pyran-2-yl)-1H-indazol-4-yl)-8-(thiazol-2-yl)quinazolin-4(3H)-one (48 mg) which was used as such in the subsequent step without further purification.

Alternatively, for many examples the nitro reduction was achieved by heating a suspension of the nitro compound (50 mg), iron (7 eq) and NH₄Cl (7 eq) in a mixture of H₂O (1 mL) and ⁿBuOH (0.5 mL) at 80° C. for 18 h. Upon cooling to rt, the reaction mixture was filtered through celite and washed with n-BuOH. The filtrate was diluted with water and was extracted with n-butanol. The organic extract was dried over anhydrous MgSO₄, filtered, and concentrated to dryness in vacuo to yield the aniline that was used without further purification in most cases.

A mixture of 5-amino-2-phenyl-6-(1-(tetrahydro-2H-pyran-2-yl)-1H-indazol-4-yl)-8-(thiazol-2-yl)quinazolin-4(3H)-one (48 mg) and concentrated HCl (0.3 mL) in dioxane (3 mL) was heated at 80° C. for 2 h. Upon cooling to rt, the reaction mixture was diluted with aqueous saturated NaHCO₃ and was extracted with n-BuOH (2×). The combined organic layers were dried over anhydrous MgSO₄, filtered and concentrated to dryness in vacuo. The residue was purified by silica gel chromatography eluting with 0-100% [10% MeOH/DCM]/DCM. The desired fractions were combined and concentrated to dryness in vacuo. The residue was lyophilized to afford 13 mg of 5-amino-6-(1H-indazol-4-yl)-2-phenyl-8-(thiazol-2-yl)quinazolin-4(3H)-one as a yellow solid. ¹H-NMR (DMSO-d₆, 400 MHz): δ 7.23 (1H, d, J=6.9 Hz), 7.51 (1H, dd, J=8.4, 7.0 Hz), 7.66-7.62 (5H, m), 7.81 (1H, d, J=3.3 Hz), 7.86 (1H, s), 8.42-8.40 (2H, m), 8.56 (1H, s), 12.65-12.59 (1H, br, S), 13.26-13.25 (1H, s). LCMS (+ESI): m/z=437.1 [M+1]⁺.

Compound 15

2-Morpholinoisonicotinoyl chloride (102 mg, 0.451 mmol) was added to a solution of 2-amino-6-nitro-5-(1-(tetrahydro-2H-pyran-2-yl)-1H-indazol-4-yl)-3-(thiazol-2-yl)benzoic acid intermediate C (70 mg, 0.150 mmol), DMAP (0.015 mmol) and triethylamine (0.612 mmol) in DMF (2 mL). The mixture was stirred for 18 h at rt. The reaction mixture was then diluted with water. The solids formed from the dilution with water were collected by filtration and air dried. The residue was purified by silica gel chromatography eluting with 0-90% CH₃CN/DCM to afford 2-(2-morpholinopyridin-4-yl)-5-nitro-6-(1-(tetrahydro-2H-pyran-2-yl)-1H-indazol-4-yl)-8-(thiazol-2-yl)-4H-benzo[d][1,3]oxazin-4-one (60 mg) LCMS (+ESI): m/z=638.7 [M+1]⁺.

A suspension of 2-(2-morpholinopyridin-4-yl)-5-nitro-6-(1-(tetrahydro-2H-pyran-2-yl)-1H-indazol-4-yl)-8-(thiazol-2-yl)-4H-benzo[d][1,3]oxazin-4-one (60 mg, 0.094 mmol), iron powder (7 eq), and NH₄Cl (7 eq) in a mixture of H₂O (2 mL) and ⁿBuOH (1 mL) was heated at 80° C. for 18 h. Iron was filtered off and the filtrate was concentrated to dryness in vacuo to afford 5-amino-2-(2-morpholinopyridin-4-yl)-6-(1-(tetrahydro-2H-pyran-2-yl)-1H-indazol-4-yl)-8-(thiazol-2-yl)-4H-benzo[d][1,3]oxazin-4-one (50 mg) which was used as such in the subsequent step without further purification. LCMS (+ESI): m/z=608.2 [M+1]⁺.

A sealable reaction tube was loaded with 5-amino-2-(2-morpholinopyridin-4-yl)-6-(1-(tetrahydro-2H-pyran-2-yl)-1H-indazol-4-yl)-8-(thiazol-2-yl)-4H-benzo[d][1,3]oxazin-4-one (50 mg) and n-BuOH (3 mL). The mixture was cooled at −78° C. and NH₃ gas was bubbled into the reaction mixture for 3 minutes. The reaction tube was sealed and was heated at 120° C. for 16 h. The reaction mixture was concentrated to dryness in vacuo to afford 5-amino-2-(2-morpholinopyridin-4-yl)-6-(1-(tetrahydro-2H-pyran-2-yl)-1H-indazol-4-yl)-8-(thiazol-2-yl)quinazolin-4(3H)-one (50 mg) which was used as such in the subsequent step without further purification. LCMS (+ESI): m/z=607.2 [M+1]⁺.

A mixture of 5-amino-2-(2-morpholinopyridin-4-yl)-6-(1-(tetrahydro-2H-pyran-2-yl)-1H-indazol-4-yl)-8-(thiazol-2-yl)quinazolin-4(3H)-one (20 mg, 0.033 mmol) and TFA (130 μL, 1.70 mmol) in DCM (3 mL) was stirred at rt for 3 h. The mixture was diluted with aqueous saturated NaHCO₃ and was extracted with n-BuOH (2×). The combined organic layers were dried over anhydrous MgSO₄, filtered, and concentrated to dryness in vacuo. Purification was performed by silica gel chromatography eluting with 5-100% CH₃CN/H₂O (+0.1% TFA). The desired fractions were combined, concentrated to dryness and lyophilized to afford 5-amino-6-(1H-indazol-4-yl)-2-(2-morpholinopyridin-4-yl)-8-(thiazol-2-yl)quinazolin-4(3H)-one (5 mg). ¹H-NMR (DMSO-d₆, 300 MHz): δ 3.61 (4H, t, J=4.6 Hz), 3.77 (4H, t, J=4.6 Hz), 7.23 (1H, d, J=7.0 Hz), 7.51 (1H, t, J=7.7 Hz), 7.63 (1H, d, J=8.4 Hz), 7.68 (1H, d, J=3.3 Hz), 7.73 (1H, d, J=5.2 Hz), 7.76 (1H, s), 7.81 (1H, d, J=3.3 Hz), 7.86 (1H, s), 8.23 (0H, s), 8.38 (1H, d, J=5.2 Hz), 8.55 (1H, s), 13.25 (1H, s). LCMS (+ESI): m/z=523.2 [M+1]⁺.

### Compounds Synthesized by Method B

### Compound 25

2-amino-3-bromo-6-nitro-5-(1-(tetrahydro-2H-pyran-2-yl)-1H-indazol-4-yl)benzoic acid intermediate D (500 mg, 1.08 mmol), 6-fluoronicotinoyl chloride (519 mg, 3.25 mmol), triethylamine (2 eq) and 4-DMAP (0.1 eq) in DMF were stirred at RT for 18 h. The solids formed from the dilution with water were collected by filtration, washed with water and acetonitrile. The residue was dried under high vacuum to afford 600 mg of 8-bromo-2-(6-fluoropyridin-3-yl)-5-nitro-6-(1-(tetrahydro-2H-pyran-2-yl)-1H-indazol-4-yl)-4H-benzo[d][1,3]oxazin-4-one which was used as such in the subsequent step without further purification. LCMS (+ESI): m/z=567.8 [M+1]⁺.

(2,4-dimethoxyphenyl)methanamine (59 mg, 0.353 mmol) was added to a solution of 8-bromo-2-(6-fluoropyridin-3-yl)-5-nitro-6-(1-(tetrahydro-2H-pyran-2-yl)-1H-indazol-4-yl)-4H-benzo[d][1,3]oxazin-4-one (200 mg, 0.353 mmol) and triethylamine (246 μL, 1.77 mmol) in THF (3 mL) at 0° C. The reaction mixture was stirred at 0° C. for 30 minutes and quenched with water. The reaction mixture was then extracted with EtOAc and n-BuOH (2×). The combined organic layers were dried over anhydrous MgSO₄, filtered and concentrated to dryness in vacuo. The residue was purified by silica gel chromatography eluting with 0-90% EtOAc/hexanes over 20 min. The desired fractions were combined and concentrated to dryness to afford 220 mg of N-(6-bromo-2-((2,4-dimethoxybenzyl)carbamoyl)-3-nitro-4-(1-(tetrahydro-2H-pyran-2-yl)-1H-indazol-4-yl)phenyl)-6-fluoronicotinamide. LCMS (+ESI): m/z=733.2/735.2 [M+1]⁺.

A mixture of N-(6-bromo-2-((2,4-dimethoxybenzyl)carbamoyl)-3-nitro-4-(1-(tetrahydro-2H-pyran-2-yl)-1H-indazol-4-yl)phenyl)-6-fluoronicotinamide (150 mg, 0.204 mmol), hexamethyldisilazane (50 mg, 0.307 mmol) and 12 (26 mg, 0.102 mmol) in DCM (5 mL) was heated to reflux for 18 h. Upon cooling the reaction mixture to rt, the volatiles were removed in vacuo. The residue was purified by silica gel chromatography eluting with 0-80% EtOAc/hexanes over 15 min. The desired fractions were combined and concentrated to dryness in vacuo to 130 mg of 8-bromo-3-(2,4-dimethoxybenzyl)-2-(6-fluoropyridin-3-yl)-5-nitro-6-(1-(tetrahydro-2H-pyran-2-yl)-1H-indazol-4-yl)quinazolin-4(3H)-one. LCMS (+ESI): m/z=715.2/717.2 [M+1]⁺.

5-amino-8-bromo-3-(2,4-dimethoxybenzyl)-2-(6-fluoropyridin-3-yl)-6-(1-(tetrahydro-2H-pyran-2-yl)-1H-indazol-4-yl)quinazolin-4(3H)-one. A suspension of 8-bromo-3-(2,4-dimethoxybenzyl)-2-(6-fluoropyridin-3-yl)-5-nitro-6-(1-(tetrahydro-2H-pyran-2-yl)-1H-indazol-4-yl)quinazolin-4(3H)-one (100 mg, 0.140 mmol)), Fe as iron powder (10 eq), ammonium chloride (10 eq), n-BuOH (1 mL) and water (2 mL) was heated at 80° C. for 18 h. Upon cooling the reaction mixture to rt, n-BuOH was added and the reaction mixture was filtered and the filtrate was concentrated to dryness in vacuo. The residue was purified by silica gel chromatography eluting with 0-80% EtOAc/hexanes over 20 min. The desired fractions were combined and concentrated to dryness in vacuo to afford 15 mg of 5-amino-8-bromo-3-(2,4-dimethoxybenzyl)-2-(6-fluoropyridin-3-yl)-6-(1-(tetrahydro-2H-pyran-2-yl)-1H-indazol-4-yl)quinazolin-4(3H)-one (LCMS (+ESI): m/z=685.2/687.2 [M+1]⁺).

Morpholine (3.2 mg, 0.036 mmol) was added to a solution of 5-amino-8-bromo-3-(2,4-dimethoxybenzyl)-2-(6-fluoropyridin-3-yl)-6-(1-(tetrahydro-2H-pyran-2-yl)-1H-indazol-4-yl)quinazolin-4(3H)-one (15 mg, 0.028 mmol) and triethylamine (4.7 mg, 0.084 mmol) in THF (1 mL). The reaction mixture was heated to reflux for 18 h. Upon cooling the reaction mixture to rt, the volatiles were removed in vacuo to afford 15 mg of 5-amino-8-bromo-2-(6-morpholinopyridin-3-yl)-6-(1-(tetrahydro-2H-pyran-2-yl)-1H-indazol-4-yl)quinazolin-4(3H)-one which was used as such in the subsequent step without further purification. LCMS (+ESI): m/z=602.2/604.2 [M+1]⁺.

5-amino-8-bromo-2-(6-morpholinopyridin-3-yl)-6-(1-(tetrahydro-2H-pyran-2-yl)-1H-indazol-4-yl)quinazolin-4(3H)-one (10 mg, 0.017 mmol) in DCM (1 mL) and TFA (50 μL) was stirred at RT for 3 h. The reaction mixture was concentrated to dryness in vacuo. Purification was performed by silica gel chromatography eluting with 0-100% CH₃CN/H₂O (+0.1% TFA) over 20 min. The desired fractions were combined, concentrated to dryness, and lyophilized to afford 3 mg of 5-amino-8-bromo-6-(1H-indazol-4-yl)-2-(6-morpholinopyridin-3-yl)quinazolin-4(3H)-one 2,2,2-trifluoroacetate as a yellow solid. ¹H-NMR (DMSO-d₆, 300 MHz): δ 3.64 (4H, s), 3.72 (4H, s), 7.01 (1H, d, J=9.2 Hz), 7.16 (1H, d, J=6.9 Hz), 7.47 (1H, t, J=7.8 Hz), 7.60 (1H, d, J=8.3 Hz), 7.67 (1H, s), 7.83 (1H, s), 8.40 (1H, d, J=9.1 Hz), 9.04 (0H, s), 12.36 (1H, s), 13.23 (1H, br s). LCMS (+ESI): m/z=518.1 [M+1]⁺.

### Compound 50

To a solution of 5-amino-8-bromo-3-[(4-methoxyphenyl)methyl]-2-(4-pyridyl)-6-(1-tetrahydropyran-2-ylindazol-4-yl)quinazolin-4-one (340 mg, 533 μmol) in dioxane (2 mL) were added (6-fluoro-2-pyridyl)boronic acid (150 mg, 1.06 mmol), ferrous; cyclopenta-2,4-dien-1-yl(diphenyl)phosphane; dichloromethane; palladium(2+); dichloride (41 mg, 50 μmol) and Sodium carbonate (2 M, 850 μL). The mixture was degassed in vacuo and then backfilled with N₂ and then it was heated at 100° C. for 1 h. The reaction was quenched with addition of water, extracted with EtOAc (3×15 mL). The combined organic extracts were washed with water and brine consecutively, dried over sodium sulfate, filtered. The filtrate was concentrated to dryness and the residue was purified by silica gel chromatography eluting with EtOAc in heptane in a gradient of 60-100% to provide 5-amino-8-(6-fluoro-2-pyridyl)-3-[(4-methoxyphenyl)methyl]-2-(4-pyridyl)-6-(1-tetrahydropyran-2-ylindazol-4-yl)quinazolin-4-one (290 mg) as an off-white solid. ESI-MS: m/z 654.4 (M+H)⁺.

To a solution of 2-morpholinoethanol (43 mg, 329 μmol) in DMF (1 mL) were added Sodium hydride (in oil dispersion) 60% dispersion in mineral oil (13 mg, 339 μmol, 60% purity) and tetrabutylammonium; iodide (2 mg, 5 μmol). The resulting mixture was stirred at rt for 10 min and then to it was added 5-amino-8-(6-fluoro-2-pyridyl)-3-[(4-methoxyphenyl)methyl]-2-(4-pyridyl)-6-(1-tetrahydropyran-2-ylindazol-4-yl)quinazolin-4-one (40 mg, 61 μmol) and the mixture was stirred at 90° C. for 1 h under N₂. After cooling to rt, the reaction was quenched by addition of AcOH. The crude reaction mixture was filtered, and the filtrate was purified by preparative HPLC (Phenomenex Gemini®) eluting with a gradient of CH₃CN (20 to 70%) in water both containing 0.1% formic acid. Appropriate fractions were combined and lyophilized to afford 5-amino-3-[(4-methoxyphenyl)methyl]-8-[6-(2-morpholinoethoxy)-2-pyridyl]-2-(4-pyridyl)-6-(1-tetrahydropyran-2-ylindazol-4-yl)quinazolin-4-one (9 mg) as an off-white solid. ESI-MS: m/z 765.4 (M+H)⁺.

The solution of 5-amino-3-[(4-methoxyphenyl)methyl]-8-[6-(2-morpholinoethoxy)-2-pyridyl]-2-(4-pyridyl)-6-(1-tetrahydropyran-2-ylindazol-4-yl)quinazolin-4-one (9 mg, 12 μmol) in TFA (1 mL) was stirred at 60° C. for 1 h. The volatiles were removed in vacuo. The residue was dissolved in MeOH and concentrated to dryness. The residue was dissolved in MeOH, treated with one drop of TEA and concentrated to dryness. The residue was dissolved in DMSO, filtered, and the filtrate was purified by preparative HPLC (Phenomenex Gemini®) eluting with a gradient of CH₃CN (10 to 60%) in water both containing 0.1% formic acid. Appropriate fractions were combined and lyophilized to afford 5-amino-6-(1H-indazol-4-yl)-8-[6-(2-morpholinoethoxy)-2-pyridyl]-2-(4-pyridyl)-3H-quinazolin-4-one (3 mg) ESI-MS: m/z 561.4 (M+H)⁺. ¹H NMR (400 MHz, DMSO-d₆) δ 13.22 (s, 1H), 12.64 (s, 1H), 8.76 (d, J=5.0 Hz, 2H), 8.15 (s, 1H), 8.13-8.06 (m, 2H), 7.95 (d, J=7.5 Hz, 1H), 7.84 (s, 1H), 7.74 (t, J=7.9 Hz, 1H), 7.57 (d, J=8.4 Hz, 1H), 7.45 (m, 1H), 7.21 (m, 1H), 6.63 (d, J=8.1 Hz, 1H), 4.32 (m, 2H), 3.36 (m, 4H), 2.56 (m, 2H), 2.24 (m, 4H)

### Compound 74

To a solution of 8-bromo-3-[(4-methoxyphenyl)methyl]-5-nitro-2-(3-pyridyl)-6-(1-tetrahydropyran-2-ylindazol-4-yl)quinazolin-4-one (50 mg, 75 μmol) in dioxane (2 mL) were added 3-cyclopropyl-1H-pyrazole (18 mg, 166 μmol), potassium carbonate (23 mg, 166 μmol) and Copper(I) iodide (14 mg, 74 μmol), N,N′-dimethylethylenediamine (8 mg, 93 μmol). The mixture was degassed in vacuo and then backfilled with N₂ and then it was heated at 110° C. for 2 days. The reaction was quenched with addition of water, extracted with EtOAc (3×15 mL). The combined organic extracts were washed with water and brine consecutively, dried over sodium sulfate, filtered. The filtrate was concentrated to dryness and the residue was purified using silica gel chromatography eluting with EtOAc in heptane (gradient 40-100%) to afford 8-(3-cyclopropylpyrazol-1-yl)-3-[(4-methoxyphenyl)methyl]-5-nitro-2-(3-pyridyl)-6-(1-tetrahydropyran-2-ylindazol-4-yl)quinazolin-4-one (30 mg) as an off-white solid. ESI-MS m/z 695.2 (M+H)⁺.

To a solution of 8-(3-cyclopropyl-1H-pyrazol-1-yl)-3-(4-methoxybenzyl)-5-nitro-2-(pyridin-3-yl)-6-(1-(tetrahydro-2H-pyran-2-yl)-1H-indazol-4-yl)quinazolin-4(3H)-one (30 mg, 43 μmol) in EtOH (4 mL)/dioxane (2 mL)/water (1 mL) were added ammonium chloride (46 mg, 860 μmol) and iron (24 mg, 430 μmol). The mixture was stirred at 100° C. for 2 h. Then the volatiles were removed in vacuo and the residue was purified using silica gel column eluting with EtOAc in heptane (gradient 40-100) to afford 5-amino-8-(3-cyclopropyl-1H-pyrazol-1-yl)-3-(4-methoxybenzyl)-2-(pyridin-3-yl)-6-(1-(tetrahydro-2H-pyran-2-yl)-1H-indazol-4-yl)quinazolin-4(3H)-one (18 mg) as an off-white solid. ESI-MS m/z 665.4 (M+H)⁺. The solution of 5-amino-8-(3-cyclopropylpyrazol-1-yl)-3-[(4-methoxyphenyl)methyl]-2-(3-pyridyl)-6-(1-tetrahydropyran-2-ylindazol-4-yl)quinazolin-4-one (18 mg, 27 μmol) in TFA (0.5 mL) was stirred at 70° C. for 2 h. The volatiles were removed in vacuo. The residue was dissolved in MeOH and concentrated to dryness. The residue was dissolved in MeOH, treated with one drop of TEA and concentrated to dryness. The residue was dissolved in DMSO, filtered, and the filtrate was purified by preparative HPLC (Phenomenex Gemini®) eluting with a gradient of CH₃CN (20 to 60%) in water both containing 0.1% formic acid. Appropriate fractions were combined and lyophilized to afford 5-amino-8-(3-cyclopropylpyrazol-1-yl)-6-(1H-indazol-4-yl)-2-(3-pyridyl)-3H-quinazolin-4-one (8 mg). ESI-MS m/z 461.3 (M+H)⁺. ¹H NMR (400 MHz, DMSO-d₆) δ 13.25-13.17 (s, 1H), 12.67 (s, 1H), 9.24 (s, 1H), 8.72 (d, J=4.7 Hz, 1H), 8.43 (d, J=8.1 Hz, 1H), 8.29 (d, J=2.0 Hz, 1H), 7.82 (s, 1H), 7.72 (s, 1H), 7.57 (m, 2H), 7.46 (t, J=7.8 Hz, 1H), 7.18 (m, 3H), 6.16 (d, J=1.8 Hz, 1H), 1.95-1.82 (m, 1H), 0.84-0.74 (m, 2H), 0.70-0.54 (m, 2H).

### Compounds Synthesized by Method C

To a solution of 6-iodo-3H-quinazolin-4-one (2.5 g, 9 mmol) in sulfuric acid (2.5 mL) were added sulfuric acid (9.2 mmol, 5 mL), nitric acid (9.2 mmol, 5 mL). The mixture was heated to 95° C. and stirred for 30 min. The mixture was cooled down and then poured onto 100 ml of ice and stirred for 30 min. The suspension was then filtered off. The solid was washed with water, dried in vacuo to provide 6-iodo-5-nitro-3H-quinazolin-4-one (2.3 g) as an of-white solid. LCMS: m/z 318.2 [M+H]⁺.

To the solution of 6-iodo-5-nitro-3H-quinazolin-4-one (22.1 g, 70 mmol) in DMF (200 mL) was carefully added sodium hydride (in oil dispersion) 60% dispersion in mineral oil (4.3 g, 112 mmol, 60% purity) in batches. The mixture was stirred at rt for 10 min and then to it was added 2-(chloromethoxy)ethyl-trimethyl-silane (18.4 g, 110 mmol). It was stirred at rt for 2 h. The reaction was quenched with NH₄Cl sat, diluted with EtOAc, washed with water and brine consecutively, dried over sodium sulfate, filtered, and concentrated to dryness. The residue was purified using silica gel chromatography eluting with EtOAc/hexanes 0-30% to provide 6-iodo-5-nitro-3-(2-trimethylsilylethoxymethyl)quinazolin-4-one (12.4 g) as an off-white solid. ¹H NMR (400 MHz, DMSO-d₆) δ 8.54 (s, 1H), 8.33 (d, J=8.7 Hz, 1H), 7.62 (d, J=8.6 Hz, 1H), 5.29 (s, 2H), 3.59-3.53 (m, 2H), 0.86-0.80 (m, 2H), −0.09 (s, 9H). LCMS: m/z 448.1 [M+H]⁺.

To the solution of 6-iodo-5-nitro-3-(2-trimethylsilylethoxymethyl)quinazolin-4-one (5 g, 11.18 mmol) in dioxane (150 mL) were added 1-tetrahydropyran-2-yl-4-(4,4,5,5-tetramethyl-1,3,2-dioxaborolan-2-yl)indazole (4.4 g, 13.4 mmol), Sodium carbonate (2 M, 11 mL) and cyclopentyl(diphenyl)phosphane; dichloropalladium; iron (800 mg, 1.1 mmol). The mixture was degassed in vacuo, backfilled with N2, and heated at 110° C. for 2 h. The volatiles were removed in vacuo. The residue was purified using silica gel chromatography eluting with EtOAc/heaxanes 20-100% to provide 5-nitro-6-(1-tetrahydropyran-2-ylindazol-4-yl)-3-(2-trimethylsilylethoxymethyl)quinazolin-4-one (4.6 g) as an off-white solid. ¹H NMR (400 MHz, DMSO-d₆) δ 8.60 (d, J=1.6 Hz, 1H), 8.09-7.93 (m, 2H), 7.89-7.78 (m, 2H), 7.46 (ddd, J=8.4, 6.9, 1.4 Hz, 1H), 7.05 (d, J=7.2 Hz, 1H), 5.89 (dd, J=9.7, 2.3 Hz, 1H), 5.34 (s, 2H), 3.94-3.81 (m, 2H), 3.73 (dt, J=11.1, 7.0 Hz, 1H), 3.66-3.56 (m, 2H), 2.46 (p, J=1.9 Hz, 2H), 2.37 (ddd, J=13.3, 9.7, 3.8 Hz, 1H), 2.08-1.91 (m, 2H), 1.71 (d, J=11.8 Hz, 1H), 1.55 (p, J=4.7, 4.2 Hz, 2H), 1.03 (d, J=1.2 Hz, 4H), 0.86 (t, J=8.0 Hz, 2H), −0.06 (d, J=1.5 Hz, 9H). LCMS: m/z 522.4 [M+H]⁺.

To a solution of 5-nitro-6-(1-tetrahydropyran-2-ylindazol-4-yl)-3-(2-trimethylsilylethoxymethyl)quinazolin-4-one (3 g, 5.8 mmol) in EtOH (50 mL) and THF (50 mL) was added palladium on carbon (1.2 g, 1.1 mmol, 10% purity). The mixture was hydrogenated using a H₂ balloon while stirred at 70° C. for 16 h. Then the mixture was filtered on Celite. The filtrate was concentrated to dryness to provide 5-amino-6-(1-tetrahydropyran-2-ylindazol-4-yl)-3-(2-trimethylsilylethoxymethyl)quinazolin-4-one (2.8 g) as an off-white solid. LCMS: m/z 492.4 [M+H]⁺.

To a solution of 5-amino-6-(1-tetrahydropyran-2-ylindazol-4-yl)-3-(2-trimethylsilylethoxymethyl)quinazolin-4-one (2.4 g, 4.9 mmol) in DCM (50 mL) was added N-Bromosuccinimide (950 mg, 5.3 mmol). The mixture was stirred at rt for 30 min. The volatiles were removed in vacuo. The residue was purified by silica gel chromatography eluting with EtOAc/hexanes 0-60% to provide 5-amino-8-bromo-6-(1-tetrahydropyran-2-ylindazol-4-yl)-3-(2-trimethylsilylethoxymethyl)quinazolin-4-one (2.1 g) as a pale yellow solid. ¹H NMR (400 MHz, Chloroform-d) δ 8.13 (s, 1H), 7.85 (d, J=1.0 Hz, 1H), 7.74 (s, 1H), 7.62 (d, J=8.4 Hz, 1H), 7.46 (dd, J=8.5, 7.0 Hz, 1H), 7.16 (dd, J=7.1, 0.8 Hz, 1H), 6.55 (s, 2H), 5.73 (dd, J=9.5, 2.6 Hz, 1H), 5.33 (s, 2H), 4.03 (d, J=10.4 Hz, 1H), 3.80-3.69 (m, 1H), 3.73-3.60 (m, 2H), 2.54 (dd, J=16.3, 7.5 Hz, 1H), 2.18-2.02 (m, 2H), 1.84-1.68 (m, 2H), 1.72-1.59 (m, 1H), 1.22 (s, OH), 1.00-0.89 (m, 2H), 0.88-0.80 (m, OH). LCMS: m/z 570.4 [M+H]⁺.

### Compound 48

In a 10 mL sealable tube, 5-amino-8-bromo-6-(1-(tetrahydro-2H-pyran-2-yl)-1H-indazol-4-yl)-3-((2-(trimethylsilyl)ethoxy)methyl)quinazolin-4(3H)-one (100 mg, 0.18 mmol) 4-flouro phenyl boronic acid (32 mg, 0.23 mmol) were dissolved in 1,4-dioxane:water (1.5 mL:0.5 mL) at room temperature followed by the addition of Cs₂CO₃ (114 mg, 0.350 mmol). PdCl₂(dppf).DCM (13 mg, 0.017 mmol) was added and the reaction mixture was purged with nitrogen for 10 min. The reaction was sealed and irradiated in microwave at 130° C. for 1 h. The reaction mixture was poured into water (30 mL) and extracted with ethyl acetate (3×15 mL). Combine organic layer was dries over sodium sulfate and concentrated under vacuum to get crude product, which was purified by silica gel chromatography eluting with 0 to 50% EtOAc in Hexanes to afford 5-amino-8-(4-fluorophenyl)-6-(1-(tetrahydro-2H-pyran-2-yl)-1H-indazol-4-yl)-3-((2-(trimethylsilyl)ethoxy)methyl)quinazolin-4(3H)-one (70 mg). LCMS: m/z 586.5 [M+H]⁺.

To the solution of 5-amino-8-(4-fluorophenyl)-6-(1-(tetrahydro-2H-pyran-2-yl)-1H-indazol-4-yl)-3-((2-(trimethylsilyl)ethoxy)methyl)quinazolin-4(3H)-one (65 mg, 0.011 mmol) in DCM (1.2 ml), TFA (1.2 ml) was added dropwise at 0° C. The resulting mixture was stirred at RT for 3 h. The reaction mixture was concentrated under vacuum and purified by using prep HPLC elution system 0.1% formic acid in water to 100% ACN. The pure product fractions were collected and lyophilized to get 5-amino-8-(4-fluorophenyl)-6-(1H-indazol-4-yl) quinazolin-4(3H)-one (5 mg). ¹H NMR (400 MHz, DMSO-d6) δ 13.22 (bs, 1H), 12.19 (bs, 1H), 8.02 (s, 1H), 7.90 (s, 1H), 7.61-7.57 (m, 3H), 7.45-7.49 (m, 2H), 7.17-7.23 (m, 3H). LCMS: m/z 372.21 [M+H]⁺.

### Compound 121

In a 30 mL sealable tube, 5-amino-8-bromo-6-(1-(tetrahydro-2H-pyran-2-yl)-1H-indazol-4-yl)-3-((2-(trimethylsilyl)ethoxy)methyl)quinazolin-4(3H)-one (100 g, 0.18 mmol) was dissolved in DMF (2 mL) at room temperature followed by the addition of tributyl(5-methylthiophen-2-yl)stannane (135 mg, 0.35 mmol) and LiCl (0.5 M in THF) (0.7 mL, 0.35 mmol). PdCl₂(dppf).DCM (7 mg, 0.018 mmol) and CuI (5 mg, 0.026 mmol) were added. The mixture was purged with nitrogen for 10 min followed and heated to 130° C. for 12 h. The reaction mixture was quenched in water (30 mL) then extracted with EtOAc (3×30 mL), then combined organic layer was dried over anhydrous Na₂SO₄, filtered and concentrated under vacuum to get crude product, which was purified by flash chromatography eluting with 30% ethyl acetate in hexane to afford 5-amino-8-(5-methylthiophen-2-yl)-6-(1-(tetrahydro-2H-pyran-2-yl)-1H-indazol-4-yl)-34(2-(trimethylsilyl)ethoxy)methyl)quinazolin-4(3H)-one (80 mg). LCMS: m/z 588 [M+H]⁺.

To the solution of 5-amino-8-(5-methylthiophen-2-yl)-6-(1-(tetrahydro-2H-pyran-2-yl)-1H-indazol-4-yl)-3-((2-(trimethylsilyl)ethoxy)methyl)quinazolin-4(3H)-one (0.065 g, 0.011 mmol) in DCM (1.2 ml), TFA (1.2 ml) was added dropwise at 0° C. The resulting mixture was stirred at RT for 3 h. The progress of the reaction was monitored by TLC using EtOAc:Hexanes (5:5) as a mobile phase. After completion of reaction, the reaction mixture was concentrated under vaccum and purified by using prep HPLC elution system 0.1% formic acid in water to 100% ACN. The desired fractions were lyophilized to afford 5-amino-6-(1H-indazol-4-yl)-8-(5-methylthiophen-2-yl)quinazolin-4(3H)-one (14 mg). LCMS: m/z 374.3 [M+H]⁺. ¹H NMR (400 MHz, DMSO d₆) δ 13.24 (s, 1H), 12.20 (s, 1H), 8.11 (s, 1H), 8.84-7.82 (m, 2H), 7.61 (d, J=8.4 Hz, 1H), 7.49 (t, J=7.2 Hz, 1H), 7.30 (d, J=3.6 Hz, 1H), 7.20 (d, J=6.8 Hz, 1H), 7.09 (bs, 2H), 6.79 (s, 1H), 2.32 (s, 3H).

### Compounds Synthesized by Method D

In a 250 mL 3 neck RBF, methyl 3-bromo-2-methylbenzoate (5 g, 21.8 mmol) was dissolved in CCl₄ (65 mL) at room temperature followed by the addition of NBS (4.47 g, 25.1 mmol) and benzolyperoxide (50 mg, 1.08 mmol) under N₂ atmosphere. The reaction mixture was stirred for 80° C. for 4 h. The reaction mixture was quenched in water (50 mL) then extracted with EtOAc (3×50 mL), then combined organic layer was dried over anhydrous Na₂SO₄, filtered and concentrated under vacuum to get crude product, which was purified by silica gel chromatography eluting with 20% ethyl acetate in hexane to afford methyl 3-bromo-2-(bromomethyl)benzoate (5.5 g). ¹H NMR (400 MHz, DMSO d₆) δ 7.94 (d, J=8.0 Hz, 1H), 7.88 (d, J=7.6 Hz, 1H), 7.43 (t, J=8.0 Hz, 1H), 5.05 (s, 2H), 3.94 (s, 3H).

In a 250 mL 3 neck RBF, methyl 3-bromo-2-(bromomethyl)benzoate (5.5 g, 17.9 mmol) was dissolved in THF (60 mL) and stirred at 0° C. To a stirred solution of methanolic ammonia solution (7 M in MeOH) (44 mL) was added and reaction mixture was stirred at room temperature for 8 h. The reaction mixture was quenched in water (70 mL) and extracted with EtOAc (3×50 mL), then combined organic layer was dried over Na₂SO₄, filtered, and concentrated under vacuum to get crude product, which was purified by trituration with n-pentane to afford 4-bromoisoindolin-1-one (2.8 g). ¹H NMR (400 MHz, DMSO d₆) δ 8.84 (s, 1H), 7.83 (d, J=8.0 Hz, 1H), 7.71 (d, J=7.6 Hz, 1H), 7.48 (t, J=7.6 Hz, 1H), 4.32 (s, 2H).

In a 100 mL of 3 neck round bottom flask, 4-bromoisoindolin-1-one (2.5 g, 11.8 mmol) was dissolved H₂SO₄ (1.5 mL, 6 v) at 0° C. and stirred for 30 min, followed by dropwise addition of concentrated HNO₃ (10 mL, 4 v). The reaction mass was stirred for 16 h at rt. The reaction mixture was poured onto chilled water (100 mL) to afford a yellow precipitate. The precipitate was recovered by filtration and air dried. The product was triturated with n-pentane to afford 4-bromo-7-nitroisoindolin-1-one (2.3 g). ¹H NMR (400 MHz, DMSO d₃) δ 9.19 (s, 1H), 8.05 (d, J=8.4 Hz, 1H), 7.88 (d, J=8.4 Hz, 1H), 4.39 (s, 2H).

In a 50 mL 3 neck round bottom flask, Fe (1.0 g, 17.9 mmol) was suspended in EtOH:H₂O (25 mL, 4:1) and stirred for 5 min. Followed addition of conc. HCl (0.1 mL, 0.1 v) and heated to 60° C. for 10 min. 4-bromo-7-nitroisoindolin-1-one (1.0 g, 3.9 mmol) was added portion wise to the reaction mass and stirred at 60° C. for 2 h. The reaction mixture was filtered through celite bed. Filtrate was collected and evaporated to afford the crude product that was triturated with hexanes and solids were dried to afford 7-amino-4-bromoisoindolin-1-one (700 mg). ¹H NMR (400 MHz, DMSO d₃) δ 8.38 (s, 1H), 7.31 (d, J=8 Hz, 1H), 6.54 (d, J=8 Hz, 1H), 6.19 (s, 2H), 4.13 (s, 2H).

In a 50 mL RBF, 7-amino-4-bromoisoindolin-1-one (0.75 g, 3.3 mmol) was dissolved in DCM (20 mL) and stirred at room temperature, followed addition of AcOH (10 mL). The reaction mixture was stirred room temperature and added NIS (0.81 g, 3.6 mmol). The reaction mixture was stirred at room temperature for 2 h. The reaction mixture was concentrated under vacuum and poured into aqueous solution of NaHCO₃ to afford a precipitate that was washed with water, filtered and dried to afford 7-amino-4-bromo-6-iodoisoindolin-1-one (0.75 g). ¹H NMR (400 MHz, DMSO d₆) δ 8.64 (s, 1H), 7.85 (s, 1H), 6.62 (s, 2H), 4.15 (s, 2H).

In a 250 mL RBF, 7-amino-4-bromo-6-iodoisoindolin-1-one (5 g, 14.1 mmol) was dissolved in DME (100 mL) and stirred at room temperature, followed addition of water (10 mL), Na₂CO₃ (4.48 g, 42.3 mmol) and intermediate A3 (14.1 mmol). The reaction mixture was purged with N₂ gas for 10 min, followed by addition of PdCl₂(dppf).DCM (2.3 g, 2.81 mmol). The reaction mixture was stirred at 100° C. for 4 h. The reaction mixture was quenched in water (30 mL) and extracted with EtOAc (3×25 mL), then combined organic layer was dried over Na₂SO₄, filtered and concentrated under vacuum to get crude product which was purified by silica gel chromatography eluting with 2% methanol in dichloromethane to afford 7-amino-4-bromo-6-(5-(methoxymethoxy)-2-methylphenyl)isoindolin-1-one (1.8 g). ¹H NMR (400 MHz, DMSO d6) δ 8.32 (s, 1H), 7.8 (bs, 1H), 6.21 (bs, 2H), 4.15 (bs, 2H), 3.57 (s, 3H), 2.31 (s, 3H).

### Compound 161

In a 250 mL RBF, 7-amino-4-bromo-6-(5-(methoxymethoxy)-2-methylphenyl)isoindolin-1-one (1 g, 2.7 mmol) was dissolved in 1,4-dioxane (12 mL) and stirred at RT, followed addition of K₂CO₃ (0.7 g, 4.0 mmol) and Bis(pinacolato)diboron (1.4 g, 5.3 mmol). The reaction mixture was purged with N₂ gas for 10 min, followed by addition of PdCl₂(dppf).DCM (0.45 g, 0.53 mmol). The reaction mixture was stirred at 110° C. for 14 h. The reaction mixture was quenched in water (30 mL) and extracted with EtOAc (3×25 mL), then combined organic layer was dried over Na₂SO₄, filtered and concentrated under vacuum to get crude product which was purified by silica gel chromatography eluting with 2% methanol in dichloromethane to afford 7-amino-6-(5-(methoxymethoxy)-2-methylphenyl)-4-(4,4,5,5-tetramethyl-1,3,2-dioxaborolan-2-yl)isoindolin-1-one (0.82 g). LCMS: m/z 425.38 [M+H]⁺.

In a 30 mL of seal tube, 2-bromo-4-chlorothiazole (0.571 mmol) was dissolved in 1, 4-dioxane (3 mL), 7-amino-6-(5-(methoxymethoxy)-2-methylphenyl)-4-(4,4,5,5-tetramethyl-1,3,2-dioxaborolan-2-yl)isoindolin-1-one (0.28 g, 0.65 mmol), Cs₂CO₃ (0.49 g, 1.5 mmol) and water (1 mL) were added at RT. The reaction mixture was purged with N₂ gas for 10 min. After completion of purging, to the reaction mixture was added PdCl₂(dppf).DCM (51 mg, 0.01 mmol) and irradiated in microwave at 100° C. for 1 h. The reaction mixture was quenched in water (30 mL) then extracted with EtOAc (3×20 mL), then combined organic layer was dried over Na₂SO₄ filtered it and concentrated under vacuum to get crude product which was purified by silica gel chromatography eluting with 3.5% methanol in dichloromethane to afford 7-amino-4-(4-chlorothiazol-2-yl)-6-(5-(methoxymethoxy)-2-methylphenyl)isoindolin-1-one (80 mg). LCMS: m/z 481.14 [M+H]⁺.

In a 10 mL RBF, 7-amino-4-(4-chlorothiazol-2-yl)-6-(5-(methoxymethoxy)-2-methylphenyl)isoindolin-1-one (80 mg) was dissolved in 1,4-dioxane (1 mL) and stirred at room temperature and followed addition of 4 M HCl in 1,4-dioxane (1 mL) at 0° C. The reaction mixture was stirred at RT for 1 h. The reaction mixture was concentrated under vacuum to get crude product which was purified by prep HPLC purification to get 7-amino-4-(4-chlorothiazol-2-yl)-6-(5-hydroxy-2-methylphenyl)isoindolin-1-one (15 mg). LCMS: m/z 372.2 [M+H]⁺. ¹H NMR (400 MHz, DMSO d₆) δ 9.39 (s, 1H), 8.61 (s, 1H), 7.67 (s, 1H), 7.54 (s, 1H), 7.16 (d, J=8.4 Hz, 1H), 6.76 (dd, J=8.4, 2.4 Hz, 1H), 6.60 (d, J=2.4 Hz, 1H), 6.16 (bs, 1H), 4.60 (s, 2H), 2.02 (s, 3H).

### Compound 163

A microwave vial was charged with 7-amino-4-bromo-6-[5-(methoxymethoxy)-2-methyl-phenyl]isoindolin-1-one (76 mg, 201 μmol), phenyl boronic acid (36 mg, 295 μmol) and cyclopentyl(diphenyl)phosphane; dichloropalladium; iron (22 mg, 30 μmol) and flushed with nitrogen. DME (2 mL) and sodium carbonate (2 M, 300 μL) was added. The vial was capped and transferred to heat block (110° C.). After 1 h 15 min, the mixture was cooled to RT, filtered on celite, washed with DCM and H₂O. The mixture was extracted with DCM two times. Combined organic extracts were concentrated and purified by silica gel chromatography eluting with a gradient of 0 to 100% EtOAc in hexanes to provide 7-amino-6-[5-(methoxymethoxy)-2-methyl-phenyl]-4-phenyl-isoindolin-1-one (62 mg) as a light amber gum. ESI-MS: m/z (M+H)⁺375.4.

To 7-amino-6-[5-(methoxymethoxy)-2-methyl-phenyl]-4-phenyl-isoindolin-1-one (62 mg, 165 μmol) in MeOH (1 mL) is added HCl in dioxane (4 M, 1 mL). After stirring for 20 min, the mixture was concentrated to dryness. The residue was taken in a minimum of DMSO and purified by preparative HPLC using a gradient of 40 to 70% acetonitrile in water (both containing 0.1% formic acid) over 12 min at a flow of 40 mL/min on a Phenomenex Gemini® 5 μm NX-C18 110 Å 150×21.2 mm column. The recovered tubes were combined and lyophilized to yield 7-amino-6-(5-hydroxy-2-methyl-phenyl)-4-phenyl-isoindolin-1-one (25 mg) as a white fluffy solid. ESI-MS: m/z (M+H)⁺331.3. ¹H NMR (400 MHz, DMSO-d₆) δ 9.30 (br s, 1H), 8.40 (s, 1H), 7.57-7.50 (m, 2H), 7.44-7.36 (m, 2H), 7.33-7.24 (m, 1H), 7.16-7.09 (m, 2H), 6.72 (dd, J=8.2, 2.6 Hz, 1H), 6.61 (d, J=2.6 Hz, 1H), 5.64 (br s, 2H), 4.57-4.35 (m, 2H), 2.02 (s, 3H).

### Compounds Synthesized by Method E

### Compound 164

To a pressure vessel containing 8-bromo-2H-isoquinolin-1-one (1.00 g, 4.46 mmol), Copper (I) iodide (90 mg, 472 μmol) and L-Proline (104 mg, 903 μmol) was added concentrated ammonium hydroxide (25 mL). The vessel was capped and stirred at 110° C. for 18 h. The mixture was cooled to RT, concentrated to dryness and the residue purified by silica gel chromatography eluting with a gradient of 0 to 10% MeOH in DCM to provide 8-amino-2H-isoquinolin-1-one (632 mg) as a greenish-gray solid. ESI-MS: m/z 161.2 (M+H)⁺. ¹H NMR (400 MHz, DMSO-d₆) δ 10.76 (br s, 1H), 7.37-7.13 (m, 3H), 6.93 (dd, J=7.1, 5.8 Hz, 1H), 6.56 (dd, J=7.7, 1.2 Hz, 1H), 6.51 (dd, J=8.1, 1.1 Hz, 1H), 6.27 (dd, J=7.1, 1.5 Hz, 1H).

To a suspension of 8-amino-2H-isoquinolin-1-one (624 mg, 3.90 mmol) in MeOH (70 mL) was added NBS (698 mg, 3.92 mmol). After stirring for 1 h 45 min, the mixture was filtrated and the solid collected was washed with a small amount of MeOH. The solid corresponds to di-bromo compound ESI-MS: m/z 319.0 (M+H)⁺ and it was discarded. The filtrate was concentrated to dryness and the residue was purified by silica gel chromatography eluting with a gradient of 0 to 80% EtOAc in hexanes. The major (more polar) product was isolated as 8-amino-5-bromoisoquinolin-1(2H)-one (479 mg) ESI-MS: m/z 239.1 (M+H)⁺. ¹H NMR (400 MHz, DMSO-d₆) δ 11.11 (br s, 1H), 7.49 (d, J=8.8 Hz, 1H), 7.13 (d, J=7.4 Hz, 1H), 6.51 (d, J=8.7 Hz, 1H), 6.46 (d, J=7.4 Hz, 1H).

To a mixture of 8-amino-5-bromo-2H-isoquinolin-1-one (140 mg, 585 μmol) and NIS (137 mg, 609 μmol) was added DCM (5 mL) and AcOH (2.5 mL). The mixture was stirred at RT for 30 min, concentrated, dried in vacuo, then the residue was triturated with saturated NaHCO₃ (20 mL), and the solid was collected by filtration. The solid was washed with water and dried in vacuo to afford 8-amino-5-bromo-7-iodo-2H-isoquinolin-1-one (185 mg) as a light beige solid. ESI-MS: m/z 367.0 (M+H)⁺. ¹H NMR (400 MHz, DMSO-d₆) δ 11.46 (br d, J=5.6 Hz, 1H), 8.04 (s, 1H), 7.26 (dd, J=7.4, 5.9 Hz, 1H), 6.52 (dd, J=7.4, 1.4 Hz, 1H).

A MW vial charged with 8-amino-5-bromo-7-iodo-2H-isoquinolin-1-one (100 mg, 274 μmol), [5-(methoxymethoxy)-2-methyl-phenyl]boronic acid (63 mg, 321 μmol), sodium carbonate (84 mg, 792.54 μmol, 33.20 μL) and Palladium(0) tetrakis(triphenylphosphine) (32 mg, 27.7 μmol) was flushed with nitrogen. DME (2 mL) and water (0.5 mL) were added. The vial was capped. The mixture was heated at 80° C. for 9 h, then left at RT for 2 days. The mixture was filtered on Celite, washed with EtOAc (10 mL) and water (5 mL). The aqueous layer was extracted with EtOAc (2×5 mL) and the combined organic extracts was washed with brine (5 mL), dried over sodium sulfate, filtered and concentrated. The residue was purified by silica gel chromatography using a gradient of 0 to 100% EtOAc in hexanes to provide 8-amino-5-bromo-7-[5-(methoxymethoxy)-2-methyl-phenyl]-2H-isoquinolin-1-one (95 mg) as a yellow solid. ESI-MS: m/z 391.3 (M+H)⁺.

A MW vial charged with Copper(I) iodide (7 mg, 36.8 μmol), Lithium Chloride (27 mg, 637 μmol) and cyclopentyl(diphenyl)phosphane; dichloropalladium; iron (18 mg, 24.6 μmol) flushed with nitrogen and 8-amino-5-bromo-7-[5-(methoxymethoxy)-2-methyl-phenyl]-2H-isoquinolin-1-one (95 mg, 244 μmol) in DMF (4 mL) followed by tributyl(thiazol-2-yl)stannane (178 mg, 477 μmol) were added. The vial was capped. The mixture was heated at 110° C. for 4 h, then cooled to RT, filtered on Celite. The solid was washed with EtOAc and the filtrate was concentrated. The residue was purified by silica gel chromatography eluting with a gradient of 0 to 100% EtOAc in hexanes to provide 8-amino-7-[5-(methoxymethoxy)-2-methyl-phenyl]-5-thiazol-2-yl-2H-isoquinolin-1-one (38 mg) as an amber gum. ESI-MS: m/z 394.4 (M+H)⁺.

To 8-amino-7-[5-(methoxymethoxy)-2-methyl-phenyl]-5-thiazol-2-yl-2H-isoquinolin-1-one (38 mg, 97 μmol) in MeOH (1 mL) was added HCl in dioxane (4 M, 1 mL). After stirring for 20 min, the mixture was concentrated and purified by preparative HPLC using a gradient of 35 to 65% acetonitrile in water (both containing 0.1% formic acid) over 12 min at a flow of 40 mL/min on a Phenomenex Gemini® 5 μm NX-C18 110 Å 150×21.2 mm column. The recovered tubes were combined and lyophilized to yield 8-amino-7-(5-hydroxy-2-methyl-phenyl)-5-thiazol-2-yl-2H-isoquinolin-1-one (10 mg, 25.92 μmol, 26.83% yield, HCl) as a light beige fluffy solid. ESI-MS: m/z 350.3 (M+H)⁺. ¹H NMR (400 MHz, DMSO-d₆) δ 11.28 (br d, J=5.7 Hz, 1H), 9.37 (br s, 1H), 7.88 (d, J=3.4 Hz, 1H), 7.68 (d, J=3.4 Hz, 1H), 7.65 (dd, J=7.5, 1.2 Hz, 1H), 7.43 (s, 1H), 7.18 (dd, J=7.5, 5.8 Hz, 1H), 7.15 (d, J=8.6 Hz, 1H), 6.75 (dd, J=8.3, 2.6 Hz, 1H), 6.61 (d, J=2.6 Hz, 1H), 2.01 (s, 3H).

### Compounds Synthesized by Method F

### Compound 134 and 22

To the solution of 5-amino-8-cyclopropyl-6-(3-methoxy-2,6-dimethyl-phenyl)-2-(4-pyridyl)-3H-quinazolin-4-one obtained from method B (28 mg, 68 μmol) in DCM (1 mL) was added boron tribromide solution (1 M, 250 μL). The mixture was stirred at rt for 2 h. The volatiles were removed in vacuo. The residue was dissolved in MeOH and evaporated to dryness. It was then dissolved in MeOH again and treated with 250 uL of triethylamine and concentrated to dryness. The residue was triturated using MeOH, filtered. The solid was washed with MeOH, dried in vacuo. The mother liquor was purified using prep-HPLC eluting with ACN/water/0.1% formic acid. The desired tubes were combined and lyophilized to provide 5-amino-8-cyclopropyl-6-(3-hydroxy-2,6-dimethyl-phenyl)-2-(4-pyridyl)-3H-quinazolin-4-one (18 mg) as an off-white solid. ESI-MS: m/z 399.2 (M+H)⁺. ¹H NMR (400 MHz, DMSO-d₆) δ 12.39 (s, 1H), 9.15 (s, 1H), 8.76 (d, J=5.0 Hz, 2H), 8.28-8.05 (m, 2H), 6.93 (d, J=8.2 Hz, 1H), 6.73 (d, J=8.2 Hz, 1H), 6.62 (s, 1H), 6.18 (s, 2H), 2.82 (m, 1H), 1.81 (s, 3H), 1.75 (s, 3H), 0.91 (m, 2H), 0.60 (m, 2H). This racemic mixture of atropisomers was separated using chiral SFC on a Mettler Toledo Minigram SFC equipped with a Phenomenex Lux Cellulose-2, 10×250 mm, 5 um column eluting 1:1 ACN/EtOH+0.1% Formic Acid (55%) at 10 mL/min). The appropriate fractions for the first peak eluting were combined, concentrated to dryness. Acetonitrile and water were added and the mixture was lyophilized to yield compound 22. ESI-MS: m/z 399.2 (M+H)⁺. ¹H NMR (400 MHz, DMSO-d₆) δ 12.39 (s, 1H), 9.15 (s, 1H), 8.76 (d, J=5.0 Hz, 2H), 8.28-8.05 (m, 2H), 6.93 (d, J=8.2 Hz, 1H), 6.73 (d, J=8.2 Hz, 1H), 6.62 (s, 1H), 6.18 (s, 2H), 2.82 (m, 1H), 1.81 (s, 3H), 1.75 (s, 3H), 0.91 (m, 2H), 0.60 (m, 2H).

### Compounds Synthesized by Method G

### Compound 165

A mixture of 2-amino-6-nitro-benzoic acid (25 g, 137 mmol) and urea (107 g, 1.78 mol) in AcOH (500 mL) was heated to reflux for 24 h. The acetic acid was evaporated under reduced pressure and the residue obtained was diluted with water (500 ml). The white precipitate was recovered by filtration, washed with water, and dried by a flow of air to yield 5-nitro-1H-quinazoline-2,4-dione (16.2 g) as a beige solid. ¹H NMR (400 MHz, DMSO-d₆) δ 11.56 (s, 2H), 7.73 (dd, J=8.4, 7.8 Hz, 1H), 7.37 (dd, J=7.8, 0.9 Hz, 1H), 7.30 (dd, J=8.4, 1.0 Hz, 1H). LCMS: m/z 206.1 [M−H]⁻.

N,N-diethylaniline (16.9 g, 113.1 mmol, 18 mL) was added to 5-nitro-1H-quinazoline-2,4-dione (10 g, 48.3 mmol) at 0° C. followed by dropwise addition of dichlorophosphorylbenzene (41.40 g, 212 mmol, 30 mL) and stirred for 30 min. The reaction mixture was heated to reflux for 18 h. The volatiles were removed in vacuo and the resulting brown thick oil was cooled to 0° C. (ice bath) and transferred slowly by portions into icy water. A brown solid formed was recovered by filtration while maintaining the temperature of the sus[ension below 4° C. The solid was washed with cold water and heptane, to yield a brown residue that was purified by silica gel chromatography eluting with a gradient of 0 to 35% EtOAc in heptane to afford 2,4-dichloro-5-nitro-quinazoline (5.7 g). NOTE: This compound is not very stable. For example, it decomposes readily in DMSO. Stability in CDCl3 is better. ¹H NMR (400 MHz, Chloroform-d) δ 7.91 (dd, J=8.3, 1.4 Hz, 1H), 7.81 (dd, J=8.3, 7.6 Hz, 1H), 7.75 (dd, J=7.6, 1.4 Hz, 1H).

Sodium methoxide, 25% in methanol (1.79 g, 8.30 mmol, 1.85 mL) was added dropwise to a suspension of 2,4-dichloro-5-nitro-quinazoline (2 g, 8.20 mmol) in MeOH (50 mL). After 15 min, a saturated solution of NH₄Cl was added, followed by water, and the white solid was recovered by filtration to afford 2-chloro-4-methoxy-5-nitro-quinazoline (1.96 g). LCMS: m/z 240.1 [M+H]⁺. ¹H NMR (400 MHz, Chloroform-d) δ 8.01 (dd, J=8.5, 1.1 Hz, 1H), 7.87 (dd, J=8.5, 7.6 Hz, 1H), 7.61 (dd, J=7.5, 1.1 Hz, 1H), 4.16 (s, 3H).

To a suspension of 2-chloro-4-methoxy-5-nitro-quinazoline (4.6 g, 19.2 mmol) and iron (5.4 g, 96.70 mmol) in EtOH (90 mL) was added ammonium chloride (5.2 g, 97.2 mmol) in water (30 mL). The reaction mixture was stirred at under reflux for 2 hrs. The mixture was filtrated on Celite while still warm and the EtOH in the filtrate was evaporated under vacuum. The remaining aqueous mixture was extracted with EtOAc (3×). The combined organic layers were washed with brine, dried over Na₂SO₄, filtered and concentrated in vacuo. The residue was purified by silica gel chromatography eluting with a gradient of 0 to 100% of EtOAc in Heptane to afford 2-chloro-4-methoxy-quinazolin-5-amine (3.9 g). LCMS: m/z 210.3 [M+H]⁺.

NBS (1.4 g, 7.9 mmol) was added to a solution of 2-chloro-4-methoxy-quinazolin-5-amine (1.6 g, 7.6 mmol) in DCM (50 mL). After 30 min, 5 g of silica was added to the mixture that was evaporated. The residue was purified by silica gel chromatography eluting with a gradient of 0 to 50% of EtOAc in hexanes. The product with the bromine para to the aniline have poor solubility and comes out of the column later and slowly (due to poor solubility). 8-bromo-2-chloro-4-methoxy-quinazolin-5-amine (1.1 g) was obtained as a beige solid. ¹H NMR (400 MHz, DMSO-d₆) δ 7.74 (d, J=8.8 Hz, 1H), 6.80 (s, 2H), 6.65 (d, J=8.8 Hz, 1H), 4.08 (s, 3H). LCMS: m/z 290.0 [M+H]⁺.

NIS (1.6 g, 7.1 mmol) was added to a solution of 8-bromo-2-chloro-4-methoxy-quinazolin-5-amine (2 g, 6.9 mmol) in DMF (30 mL) and the mixture was stirred for 3 h at 70° C. After cooling to RT, water was added, and the mixture was extracted with EtOAc twice. The organic layer was washed with brine, dried over Na₂SO₄ and purified by silica gel chromatography using a gradient of 0 to 50% of EtOAc in hexanes to yield 8-bromo-2-chloro-6-iodo-4-methoxy-quinazolin-5-amine (2.1 g) as a beige solid. LCMS: m/z 416.0 [M+H]⁺.

In a 10 mL sealable tube containing a solution of 8-bromo-2-chloro-6-iodo-4-methoxyquinazolin-5-amine (0.4 g, 0.96 mmol) and 1-(tetrahydro-2H-pyran-2-yl)-4-(4,4,5,5-tetramethyl-1,3,2-dioxaborolan-2-yl)-1H-indazole (0.348 g, 1.06 mmol) in dioxane (10 mL) was added water (0.8 mL) and Cs₂CO₃ (0.78 g, 2.4 mmol). The mixture was purged with argon gas for 15 min followed by addition of PdCl₂(dppf).DCM (0.078 g, 0.096 mmol). The reaction tube was sealed and the mixture was stirred at 110° C. for 4 h. The reaction mixture was poured in to water (30 mL) and extracted with ethyl acetate (3×15 mL). Combine organic layer was dried over sodium sulfate and concentrated under vacuum to get crude product, which was purified by silica gel chromatography eluting with a gradient of 0 to 50% EtOAc in Hexanes to yield 8-bromo-2-chloro-4-methoxy-6-(1-(tetrahydro-2H-pyran-2-yl)-1H-indazol-4-yl)quinazolin-5-amine (0.290 g). LCMS: m/z 488.2 [M+H]⁺.

To the stirred solution of 8-bromo-2-chloro-4-methoxy-6-(1-(tetrahydro-2H-pyran-2-yl)-1H-indazol-4-yl)quinazolin-5-amine (0.25 g, 0.51 mmol) in DMF (2.5 mL) was added K₂CO₃ (0.211 g, 1.53 mmol) and phenol (0.072 g, 0.76 mmol). The resulting mixture was irradiated in Microwave at 130° C. for 1 h. The reaction mixture was poured in to water (10 mL) and extracted with ethyl acetate (3×10 mL). Combine organic layer was dried over sodium sulfate and concentrated under vacuum to get crude product, which was purified by silica gel chromatography eluting with a gradient of 0 to 30% EtOAc in Hexanes to afford 8-bromo-4-methoxy-2-phenoxy-6-(1-(tetrahydro-2H-pyran-2-yl)-1H-indazol-4-yl) quinazolin-5-amine (0.140 g). LCMS: m/z 532.6 [M+H]⁺.

In a 10 mL sealable tube, 8-bromo-4-methoxy-2-phenoxy-6-(1-(tetrahydro-2H-pyran-2-yl)-1H-indazol-4-yl)quinazolin-5-amine (0.200 g, 0.36 mmol) and 2-(tributylstannyl)thiazole (0.205 g, 0.54 mmol) were dissolved in DMF (2 mL) at room temperature reaction mixture was purged with nitrogen for 15 min. CuI (0.01 g, 0.054 mmol), LiCl (0.030 g, 0.73 mmol) and PdCl₂(dppf).DCM (0.029 mg, 0.036 mmol) were added. The tube was sealed, and the reaction mixture was stirred at 100° C. for 16 hour. The reaction mixture was poured into water and extracted with ethyl acetate (3×10 mL). Combine organic layer was dried over sodium sulfate and concentrated under vacuum to get crude product, which was purified by silica gel chromatography eluting with a gradient of 0 to 40% ethyl acetate in hexanes to afford 4-methoxy-2-phenoxy-6-(1-(tetrahydro-2H-pyran-2-yl)-1H-indazol-4-yl)-8-(thiazol-2-yl)quinazolin-5-amine (0.046 g) LCMS: m/z 537.4 [M+H]⁺.

To the solution of 5-amino-2-phenoxy-6-(1-(tetrahydro-2H-pyran-2-yl)-1H-indazol-4-yl)-8-(thiazol-2-yl)quinazolin-4(3H)-one (0.03 g, 0.050 mmol) in DCM (0.6 mL), TFA (0.3 mL) was added dropwise at 0° C. The resulting mixture was stirred at RT for 1 h. The reaction mixture was concentrated under vacuum. Residue was dissolve in water, pH was adjusted to neutral to basic with saturated NaHCO₃ solution and extracted with EtOAc (3×10 mL) combine organic layer was collected and evaporated under vacuum to obtain crude which was purified by silica gel chromatography eluting with 3% MeOH in DCM to yield 15 mg of a compound having LCMS purity 50% which was further purified by using prep HPLC elution system 0.1% formic acid in water to 100% ACN. The pure product fractions were collected and lyophilized to yield 5-amino-6-(1H-indazol-4-yl)-2-phenoxy-8-(thiazol-2-yl)quinazolin-4(3H)-one (2.8 mg). ¹H NMR (400 MHz, DMSO-d₆) δ 13.24 (s, 1H), 12.93 (bs, 1H), 8.37 (s, 1H), 7.79 (s, 1H), 7.60-7.58 (m, 2H), 7.56 (t, J=7.6 Hz, 2H), 7.47 (t, J=8 Hz, 2H), 7.40-7.37 (m 3H), 7.19-7.15 (m, 2H). LCMS: m/z 453.3 [M+H]⁺.

### Chiral Separation of Selected Compounds

Racemic mixtures of atropisomers were separated using chiral SFC methods on, for example, a Mettler Toledo Minigram SFC (MTM), a Waters Prep 15 SFC-MS (WP15), a Waters Prep 100 SFC-MS (WP100) or a Pic Solution Hybrid 10-150 (PSH). An appropriate column was selected to achieve a satisfactory resolution of the peaks. The appropriate fractions for each peak were combined, concentrated and usually taken in a mixture of water and a suitable water miscible organic solvent such as EtOH, IPA, CH₃CN or a mixture thereof and freeze-dried. The separated products were reanalyzed by chiral SFC to assess chiral purity.

Exemplary columns used for chiral separations include, for example, Phenomenex Lux Cellulose-2, 10×250 mm, 5 μm; Phenomenex Lux Cellulose-2, 30×250 mm, 5 μm; Chiral Technologies IA, 10×250 mm, 5 μm; Chiral Technologies IC, 10×250 mm, 5 μm; Chiral Technologies ID, 10×250 mm, 5 μm; Chiral Technologies IG, 10×250 mm, 5 μm; Chiral Technologies AS, 10×250 mm, 5 μm; Phenomenex Lux Cellulose-4, 10×250 mm, 5 μm; Phenomenex Lux Cellulose-1, 21.2×250 mm, 5 μm.

Structural assignments of the separated atropisomers were confirmed by biological activity where the biologically active enantiomer was assigned to have the (S) configuration, which was confirmed by X-ray crystallography of key compounds.

### Example 2. Enzymatic Assay

Detection of Myt1 kinase activity utilized a recombinant human Myt1 kinase assay measuring the hydrolysis of ATP using a commercially available ADP-Glo Assay (ADP-Glo™ Kinase Assay from Promega, 10 000 assays, #V9102). Briefly, 5 μL recombinant human Myt1 (full length PKMYT1 recombinant human protein expressed in insect cells from Thermo Fisher #A33387; ˜80% purity) was prepared in reaction buffer (70 mM HEPES, 3 mM MgCl₂, 3 mM MnCl₂, 50 μg/ml PEG 20000, 3 μM Na-orthovanadate, 1.2 mM DTT) and added to 384 well white polystyrene, flat bottom well, non-treated, microplate (Corning #3572). After this, 5 μL of compounds (diluted in reaction buffer to 0.5% DMSO) was added to the microplate and the plate was spun briefly and incubated at 22° C. for 15 minutes. Ultra-Pure Adenosine Triphosphate (ATP) solution (ADP-Glo kit from Promega) was diluted in reaction buffer and 5 μL was added to the microplate, spun down briefly and incubated for 60 minutes at 30° C. The final Myt1 enzyme concentration was 18 nM and the final ATP concentration was 10 μM. After the 60-minute incubation, 15 μL of ADP-Glo reagent was added and the plate was spun briefly and sealed and incubated in the dark for 40 minutes at 22° C. Following this, 30 μL of kinase detection reagent was added per well and the plate was spun briefly, sealed and incubated for 45-60 minutes at 22° C. in the dark. Luminescence was read using the Envision (250 ms integration). The IC₅₀ and the % max inhibition were calculated for each inhibitor compound tested.

Exemplary prepared compounds and their activities are shown in Table 2 below. In Table 2, the Method column indicates a preparatory method described above used in the preparation of the compounds.

TABLE 2

| | | MS (+ESI) | | |
|---|---|---|---|---|
| | | [M + 1] | | Myt1 IC₅₀ |
| Compound | method | (m/z) | 1H NMR | (nM) |
| 1 | A | 437.1 | ¹H-NMR (DMSO-d₆, 400 MHz): δ 7.23 (1H, d, J = | 4 |
| | | | 6.9 Hz), 7.51 (1H, dd, J = 8.4, 7.0 Hz), 7.66-7.62 | |
| | | | (5H, m), 7.81 (1H, d, J = 3.3 Hz), 7.86 (1H, s), | |
| | | | 8.42-8.40 (2H, m), 8.56 (1H, s), 12.65-12.59 (1H, | |
| | | | br, S), 13.26-13.25 (1H, s). | |
| 2 | A | 522.9 | ¹H-NMR (DMSO-d₆, 300 MHz): δ 3.69 (8H, dd, J = | 15 |
| | | | 19.1, 5.2 Hz), 7.04 (1H, d, J = 9.1 Hz), 7.22 | |
| | | | (1H, d, J = 6.9 Hz), 7.50 (1H, t, J = 7.5 Hz), 7.66- | |
| | | | 7.58 (2H, m), 7.79 (1H, d, J = 3.3 Hz), 7.85 (1H, | |
| | | | s), 8.39 (1H, s), 8.59-8.50 (2H, m), 9.22 (1H, d, J = | |
| | | | 2.5 Hz), 13.26 (1H, s). | |
| 3 | A | 451.1 | ¹H-NMR (DMSO-d₆, 300 MHz): δ 2.44 (3H, s), | 33 |
| | | | 7.23 (1H, d, J = 7.2 Hz), 7.53-7.42 (4H, m), 7.63 | |
| | | | (2H, m), 7.80 (1H, d, J = 3.5 Hz), 7.86 (1H, s), | |
| | | | 8.32 (2H, d, J = 8.0 Hz), 8.55 (1H, s), 13.26 (1H, s). | |
| 4 | A | 453.1 | ¹H-NMR (DMSO-d₆, 300 MHz): δ 6.53 (1H, s), | 5 |
| | | | 6.61 (1H, d, J = 8.8 Hz), 6.79 (2H, s), 7.21 (1H, | |
| | | | d, J = 6.9 Hz), 7.50 (1H, t, J = 7.7 Hz), 7.64-7.61 | |
| | | | (2H, m), 7.79 (1H, d, J = 3.3 Hz), 7.85 (1H, s), | |
| | | | 8.39 (1H, dd, J = 8.8, 2.5 Hz), 8.53 (1H, s), 9.01 | |
| | | | (1H, d, J = 2.4 Hz), 12.32 (1H, s), 13.28 (1H, s). | |
| 5 | A | 461.9 | ¹H-NMR (DMSO-d₆, 300 MHz): δ 7.23-7.18 (1H, | 89 |
| | | | m), 7.57-7.42 (4H, m), 7.74-7.71 (1H, m), 7.86- | |
| | | | 7.82 (1H, m), 7.97-7.92 (2H, m), 8.42 (1H, t, J = | |
| | | | 0.7 Hz), 8.81-8.75 (2H, m), 13.20-13.14 (1H, m). | |
| 6 | A | 513.1 | ¹H-NMR (DMSO-d₆, 300 MHz): δ 7.23 (1H, d, J = | >2000 |
| | | | 7.1 Hz), 7.63-7.42 (7H, m), 7.94-7.81 (7H, m), | |
| | | | 8.56 (3H, d, J = 10.7 Hz), 13.24 (1H, s). | |
| 7 | A | 449.1 | ¹H-NMR (DMSO-d₆, 300 MHz): δ 6.55 (1H, d, J = | 4 |
| | | | 9.0 Hz), 6.74 (2H, s), 7.15 (1H, d, J = 7.1 Hz), | |
| | | | 7.47 (1H, t, J = 8.1 Hz), 7.59 (1H, d, J = 8.8 Hz), | |
| | | | 7.65 (1H, s), 7.83 (1H, s), 8.25 (2H, s), 8.87 (1H, | |
| | | | s), 13.23 (1H, br s). | |
| 8 | A | 401.1 | ¹HNMR (DMSO-d6, 400 MHz): δ 1.23 (3H, m), | 131 |
| | | | 1.38 (2H, m), 7.18 (1H, d, J = 6.9 Hz), 7.48 (1H, | |
| | | | t, J = 7.6 Hz), 7.61-7.57 (2H, m), 7.76 (1H, d, J = | |
| | | | 3.3 Hz), 7.81 (1H, s), 8.48 (1H, s), 12.64 (1H, s), | |
| | | | 13.24 (1H, s). | |
| 9 | A | 375.1 | ¹H-NMR (DMSO-d6, 400 MHz): δ 2.48 (5H, s), | 47 |
| | | | 7.19 (1H, d, J = 7.0 Hz), 7.49 (1H, t, J = 7.6 Hz), | |
| | | | 7.57 (1H, d, J = 3.3 Hz), 7.61 (1H, d, J = 8.4 Hz), | |
| | | | 7.76 (1H, d, J = 3.3 Hz), 7.82 (1H, s), 8.50 (1H, | |
| | | | s), 12.33 (1H, s), 13.25 (1H, s). | |
| 10 | B | 517.1 | 1H NMR (DMSO- d 6, 400 MHz): δH 2.92 (4H, | 3 |
| | | | m), 3.69 (4H, m), 7.03-6.99 (1H, d), 7.15 (1H, d, | |
| | | | J = 7.0 Hz), 7.47 (1H, t, J = 7.7 Hz), 7.60 (1H, d, | |
| | | | J = 8.4 Hz), 7.67 (1H, s), 7.83 (1H, s), 8.20 (1H, | |
| | | | s), 8.38 (1H, dd, J = 9.1, 2.5 Hz), 9.03 (1H, d, J = | |
| | | | 2.4 Hz), 13.27-13.18 (1H, s, b). | |
| 11 | B | 571.1 | 1H NMR (DMSO- d 6, 400 MHz): δH 0.67 (2H, | 5 |
| | | | m), 0.72 (2H, m), 2.79 (1H, m), 3.39 (2H, m), | |
| | | | 3.89 (2H, m), 4.19 (2H, s), 6.96 (1H, d, J = 9.3 | |
| | | | Hz), 7.15 (1H, d, J = 7.0 Hz), 7.47 (1H, t, J = 8.0 | |
| | | | Hz), 7.59 (1H, d, J = 8.5 Hz), 7.64 (1H, s), 7.83 | |
| | | | (1H, s), 8.42 (1H, d, J = 9.6 Hz), 9.05 (1H, s), | |
| | | | 13.24 (1H, s). | |
| 12 | B | 476.1 | 1H NMR (DMSO- d 6, 400 MHz): δH 3.14 (6H, | 9 |
| | | | s), 6.79 (1H, d, J = 9.1 Hz), 7.15 (1H, d, J = 7.0 | |
| | | | Hz), 7.49-7.45 (1H, m), 7.59 (1H, d, J = 8.4 Hz), | |
| | | | 7.65 (1H, s), 7.83 (1H, s), 8.38 (1H, d), 9.03 (1H, | |
| | | | s), 13.23 (1H, s). | |
| 13 | A | 444.9 | ¹H-NMR (DMSO-d₆, 300 MHz): δ 2.13-1.89 (4H, | 1410 |
| | | | m), 3.02-2.88 (2H, m), 4.08-3.99 (2H, m), 6.54 | |
| | | | (1H, s), 7.19 (1H, d, J = 7.7 Hz), 7.55-7.45 (2H, | |
| | | | m), 7.65-7.57 (2H, m), 7.85-7.75 (2H, m), 8.52 | |
| | | | (1H, s), 13.25 (1H, s). | |
| 14 | A | 515.1 | ¹H-NMR (DMSO-d₆, 300 MHz): δ 3.33 (3H, s), | 6 |
| | | | 7.22 (1H, d, J = 7.0 Hz), 7.50 (1H, t, J = 7.6 Hz), | |
| | | | 7.61 (2H, m), 7.86-7.80 (2H, m), 8.14 (2H, d, J = | |
| | | | 8.0 Hz), 8.22 (1H, s), 8.53 (1H, s), 8.66 (2H, d, J = | |
| | | | 7.9 Hz), 13.24 (1H, s). | |
| 15 | A | 523.1 | ¹H-NMR (DMSO-d₆, 300 MHz): δ 3.61 (4H, t, J = | 10 |
| | | | 4.6 Hz), 3.77 (4H, t, J = 4.6 Hz), 7.23 (1H, d, J = | |
| | | | 7.0 Hz), 7.51 (1H, t, J = 7.7 Hz), 7.63 (1H, d, J = | |
| | | | 8.4 Hz), 7.68 (1H, d, J = 3.3 Hz), 7.73 (1H, d, J = | |
| | | | 5.2 Hz), 7.76 (1H, s), 7.81 (1H, d, J = 3.3 Hz), | |
| | | | 7.86 (1H, s), 8.23 (OH, s), 8.38 (1H, d, J = 5.2 | |
| | | | Hz), 8.55 (1H, s), 13.25 (1H, s). | |
| 16 | A | 452.1 | ¹H-NMR (DMSO-d₆, 300 MHz): δ 6.71 (2H, d, J = | 54 |
| | | | 8.4 Hz), 7.21 (1H, d, J = 7.1 Hz), 7.50 (1H, t, J = | |
| | | | 0.1 Hz), 7.66-7.60 (2H, m), 7.81 (1H, s), 7.86 | |
| | | | (1H, s), 8.19 (2H, d, J = 8.2 Hz), 8.52 (1H, s), | |
| | | | 12.15 (1H, br, s), 13.25 (1H, br, s). | |
| 17 | A | 494.1 | ¹H-NMR (DMSO-d₆, 300 MHz): δ 2.13 (3H, s), | 78 |
| | | | 7.22 (1H, d, J = 7.1 Hz), 7.51 (1H, t, J = 8.0 Hz), | |
| | | | 7.69-7.60 (2H, m), 7.89-7.81 (4H, m), 8.38 (2H, | |
| | | | d, J = 8.4 Hz), 8.55 (1H, s), 10.33 (1H, s), 12.50 | |
| | | | (1H, s), 13.26 (1H, br s). | |
| 18 | A | 521.9 | ¹H-NMR (DMSO-d₆, 300 MHz): δ 3.35 (4H, m), | 9 |
| | | | 3.83 (4H, m), 7.23 (2H, d, J = 7.5 Hz), 7.49 (2H, | |
| | | | q, J = 8.3 Hz), 7.63 (1H, d, J = 8.4 Hz), 7.68 (1H, | |
| | | | d, J = 3.5 Hz), 7.81 (1H, d, J = 3.4 Hz), 7.86 (1H, | |
| | | | s), 7.98-7.91 (2H, m), 8.33 (1H, s), 8.55 (1H, s), | |
| | | | 13.26 (1H, br, s). | |
| 19 | B | 518.1 | 1H NMR (DMSO- d 6, 400 MHz): dH 1.87-1.80 | 3 |
| | | | (1H, m), 2.26-2.19 (1H, m), 3.57 (1H, dd, J = 8.9, | |
| | | | 3.8 Hz), 3.76-3.71 (1H, m), 3.92-3.83 (2H, m), | |
| | | | 4.53- 4.46 (1H, m), 6.63 (1H, d, J = 8.9 Hz), 7.15 | |
| | | | (1H, d, J = 7.0 Hz), 7.46 (1H, t, J = 7.7 Hz), 7.55 | |
| | | | (1H, d, J = 6.3 Hz), 7.59 (1H, d, J = 8.4 Hz), 7.65 | |
| | | | (1H, s), 7.83 (1H, s), 8.26-8.23 (1H, m), 8.37 (1H, | |
| | | | s), 8.94 (1H, d, J = 2.4 Hz), 12.35-12.24 (1H, | |
| | | | m) 13.26-13.21 (1H, s). | |
| 20 | A | 450.9 | ¹H-NMR (DMSO-d₆, 300 MHz): δ 2.47 (3H, s), | 10 |
| | | | 7.23 (1H, d, J = 6.9 Hz), 7.55-7.45 (3H, m), 7.65- | |
| | | | 7.62 (1H, m), 7.69 (1H, d, J = 3.4 Hz), 7.83 (1H, | |
| | | | d, J = 3.4 Hz), 7.87 (1H, s), 8.25-8.18 (2H, m), | |
| | | | 8.56 (1H, s), 12.57 (1H, br s), 13.25 (1H, br s). | |
| 21 | A | 462.1 | ¹H-NMR (DMSO-d₆, 300 MHz): δ 7.23 (1H, d, J = | 6 |
| | | | 7.0 Hz), 7.51 (1H, t, J = 7.5 Hz), 7.69-7.62 (2H, | |
| | | | m), 7.86-7.82 (3H, m), 8.13 (1H, d, J = 7.5 Hz), | |
| | | | 8.57 (1H, s), 8.74-8.70 (2H, m), 13.26 (1H, s). | |
| | | | LCMS (+ESI): m/z = 462.1 [M + 1]⁺. | |
| 22 | B + F | 399.3 | 1H NMR (400 MHz, DMSO-d6) δ 12.39 (s, 1H), | 3 |
| | | | 9.15 (s, 1H), 8.76 (d, J = 5.0 Hz, 2H), 8.28-8.05 | |
| | | | (m, 2H), 6.93 (d, J = 8.2 Hz, 1H), 6.73 (d, J = 8.2 | |
| | | | Hz, 1H), 6.62 (s, 1H), 6.18 (s, 2H), 2.82 (m, 1H), | |
| | | | 1.81 (s, 3H), 1.75 (s, 3H), 0.91 (m, 2H), 0.60 (m, 2H). | |
| 23 | B | | 1H NMR (DMSO- d 6, 400 MHz): δH 2.48 (5H, | 4 |
| | | | s), 7.19 (1H, d, J = 7.0 Hz), 7.49 (1H, t, J = 7.6 | |
| | | | Hz), 7.57 (1H, d, J = 3.3 Hz), 7.61 (1H, d, J = 8.4 | |
| | | | Hz), 7.76 (1H, d, J = 3.3 Hz), 7.82 (1H, s), 8.50 | |
| | | | (1H, s), 12.33 (1H, s), 13.25 (1H, s). | |
| 24 | B | 531.1 | ¹H-NMR (DMSO-d₆, 300 MHz): δ 2.23 (3H, s), | 10 |
| | | | 2.41 (4H, t, J = 5.0 Hz), 3.67 (4H, t, J = 4.7 Hz), | |
| | | | 7.00 (1H, d, J = 9.2 Hz), 7.16 (1H, d, J = 6.9 Hz), | |
| | | | 7.47 (1H, t, J = 7.6 Hz), 7.60 (1H, d, J = 8.5 Hz), | |
| | | | 7.66 (1H, s), 7.83 (1H, s), 8.17 (1H, s), 8.37 (1H, | |
| | | | d, J = 9.0 Hz), 9.02 (1H, s), 12.41 (1H, br, s), | |
| | | | 13.23 (1H, s). | |
| 25 | B | 518.1 | ¹H-NMR (DMSO-d6, 300 MHz): δ 3.64 (4H, s), | 23 |
| | | | 3.72 (4H, s), 7.01 (1H, d, J = 9.2 Hz), 7.16 (1H, | |
| | | | d, J = 6.9 Hz), 7.47 (1H, t, J = 7.8 Hz), 7.60 (1H, | |
| | | | d, J = 8.3 Hz), 7.67 (1H, s), 7.83 (1H, s), 8.40 | |
| | | | (1H, d, J = 9.1 Hz), 9.04 (0H, s), 12.36 (1H, s), | |
| | | | 13.23 (1H, br s). | |
| 26 | B | 557.1 | 1H NMR (DMSO- d 6, 400 MHz): δH 0.86 (2H, t, | 5 |
| | | | J = 6.9 Hz), 1.03-0.95 (2H, m), 3.02-2.86 (1H, | |
| | | | m), 3.35-3.23 (4H, m), 3.71-3.55 (2H, m), 4.73- | |
| | | | 4.51 (2H, m), 7.17-7.13 (2H, m), 7.47 (1H, t, J = | |
| | | | 7.7 Hz), 7.60 (1H, d, J = 8.4 Hz), 7.68 (1H, s), | |
| | | | 7.83 (1H, s), 8.46 (1H, d, J = 9.0 Hz), 9.08 (1H, | |
| | | | s), 12.45-12.45 (1H, m), 3.32-13.20 (1H, m). | |
| 27 | B + F | 465.3 | 1H NMR (400 MHz, DMSO-d6) δ 12.48 (s, 1H), | 2 |
| | | | 9.17 (s, 1H), 8.87-8.71 (m, 2H), 8.26 (s, 1H), | |
| | | | 8.10-8.02 (m, 3H), 7.45 (s, 1H), 6.96 (d, J = 8.2 | |
| | | | Hz, 1H), 6.76 (d, J = 8.1 Hz, 1H), 6.46 (bs, 1H), | |
| | | | 3.70 (m, 1H), 1.88 (s, 3H), 1.81 (s, 3H), 1.08- | |
| | | | 0.96 (m, 2H), 0.96-0.82 (m, 2H). | |
| 28 | C | 361.1 | ¹H-NMR (DMSO-d₆, 400 MHz): δ 7.21-7.19 (1H, | 4 |
| | | | m), 7.49 (1H, t, J = 7.7 Hz), 7.62-7.57 (2H, m), | |
| | | | 7.77 (1H, d, J = 3.3 Hz), 7.83 (1H, s), 8.28 (1H, | |
| | | | s), 8.44 (1H, s), 8.52 (1H, s), 13.29-13.25 (1H, m). | |
| 29 | B | 506.1 | 1H NMR (DMSO- d 6, 400 MHz): dH 3.29 (4H, | 3 |
| | | | s), 3.54-3.50 (4H, m), 6.64 (1H, d, J = 9.0 Hz), | |
| | | | 7.15 (1H, d, J = 7.0 Hz), 7.37 (1H, m), 7.46 (1H, | |
| | | | t, J = 7.7 Hz), 7.59 (1H, d, J = 8.4 Hz), 7.65 (1H, | |
| | | | s), 7.83 (1H, s), 8.23 (1H, d, J = 9.0 Hz), 8.37 | |
| | | | (1H, s), 8.92 (1H, s), 12.25 (1H, br s), 13.23 (1H, s). | |
| | | | 8.51 (s, 1H), 7.46 (dd, J = 10.2, 1.8 Hz, 2H), 7.29 | |
| | | | (d, J = 8.5 Hz, 1H), 7.22 (s, 1H), 5.56 (s, 2H), | |
| | | | 4.22 (d, J = 1.1 Hz, 2H), 2.14 (s, 3H). | |
| 31 | B | 522.2 | ¹H-NMR (DMSO-d₆, 300 MHz): δ 3.27 (4H, t, J = | 15 |
| | | | 5.1 Hz), 3.89 (4H, t, J = 4.9 Hz), 7.23 (1H, d, J = | |
| | | | 7.0 Hz), 7.52 (1H, t, J = 7.7 Hz), 7.65 (1H, m), | |
| | | | 7.70 (1H, d, J = 3.3 Hz), 7.78-7.76 (2H, m), 7.86- | |
| | | | 7.84 (2H, m), 8.44 (1H, d, J = 5.2 Hz), 8.58 (1H, | |
| | | | s), 8.83 (1H, s), 12.73 (1H, s), 13.27 (1H, s). | |
| 32 | B | 559.9 | 1H NMR (400 MHz, DMSO): ? 9.01 (s; 1 H); 8.35 | 6 |
| | | | (d; J = 9.30 Hz; 1 H); 7.82 (s; 1 H); 7.62 (s; 1 H); | |
| | | | 7.56-7.59 (m; 1 H); 7.43-7.48 (m; 1 H); 7.13-7.16 | |
| | | | (m; 1 H); 6.97-7.01 (m; 1 H); 4.29-4.36 (m; 2 H); | |
| | | | 3.04-3.12 (m; 2 H); 1.84-1.90 (m; 2 H); 1.48-1.53 | |
| | | | (m; 1 H); 1.14-1.24 (m; 1 H). | |
| 33 | A | 437.9 | ¹H-NMR (DMSO-d₆, 300 MHz): δ 13.32 (br s; 1 | 9 |
| | | | H); 12.88 (br s; 1 H); 8.57 (s; 1 H); 7.86 (d; J = | |
| | | | 2.1 Hz; 2 H); 7.65 (m; 2 H); 7.51 (m. 2 H); 7.22 | |
| | | | (m, 3 H). | |
| 34 | B | 558.9 | 1H NMR (400 MHz, DMSO): ? 8.99 (d; J = 2.34 | 10 |
| | | | Hz; 1 H); 8.34 (dd; J = 9.20; 2.25 Hz; 1 H); 7.81 | |
| | | | (s; 1 H); 7.63 (s; 1 H); 7.57 (d; J = 8.38 Hz; 1 H); | |
| | | | 7.45 (t; J = 7.67 Hz; 1 H); 7.13 (d; J = 7.04 Hz; 1 | |
| | | | H); 6.99-7.01 (m; 1 H); 4.35 (s; 4 H); 3.62 (t; J = | |
| | | | 5.11 Hz; 4 H); 1.82 (t; J = 5.10 Hz; 4 H). | |
| 35 | B | 504.1 | 1H NMR (400 MHz, DMSO): 8.92 (s; 1 H); 8.55- | 29 |
| | | | 8.59 (m; 1 H); 8.14 (s; 1 H); 7.80 (d; J = 1.42 Hz; | |
| | | | 1 H); 7.54-7.57 (m; 2 H); 7.42-7.46 (m; 1 H); 7.12 | |
| | | | (d; J = 6.99 Hz; 1 H); 6.99-7.02 (m; 1 H); 5.19- | |
| | | | 5.23 (m; 1 H); 4.67-4.73 (m; 1 H); 4.43-4.49 (m; 1 | |
| | | | H); 4.33-4.39 (m; 1 H); 1.21-1.23 (m; 1 H) | |
| 36 | B | 561.2 | ¹H-NMR (DMSO-d₆, 400 MHz): δ 9.01 (s; 1 H); | 76 |
| | | | 8.35-8.37 (m; 1 H); 7.81 (s; 1 H); 7.62 (s; 1 H); | |
| | | | 7.57 (dd; J = 8.24; 1.75 Hz; 1 H); 7.46 (dd; J = | |
| | | | 7.26; 1.61 Hz; 1 H); 7.13-7.15 (m; 1 H); 6.94-6.97 | |
| | | | (m; 1 H); 3.63 (d; J = 5.34 Hz; 8 H); 3.51-3.55 (m; 4 H). | |
| 37 | B | 506.1 | 1H NMR (400 MHz, DMSO): 1H NMR (400 MHz, | 11 |
| | | | DMSO): ? 8.13 (d; J = 5.38 Hz; 1 H); 7.82 (s; 1 | |
| | | | H); 7.70 (s; 1 H); 7.59-7.61 (m; 1 H); 7.46 (dd; J = | |
| | | | 8.37; 7.07 Hz; 1 H); 7.27 (s; 1 H); 7.20-7.21 (m; | |
| | | | 1 H); 7.15 (d; J = 7.08 Hz; 1 H); 6.90 (br; 1 H); | |
| | | | 3.47-3.48 (m; 4 H); 3.28 (s; 3 H); | |
| 38 | B | 531.1 | ¹H-NMR (DMSO-d₆, 400 MHz): δ 8.28 (s; 1 H); | 10 |
| | | | 7.82 (s; 1 H); 7.71 (s; 1 H); 7.62 (s; 2 H); 7.47 (s; | |
| | | | 1 H); 7.16 (s; 1 H); 3.62 (s; 4 H); 3.30 (s; 3 H) | |
| | | | 2.27 (s; 4 H). | |
| 39 | A | 537 | ¹H-NMR (DMSO-d₆, 300 MHz): δ 13.27 (s; 1 H); | 8 |
| | | | 8.55-8.56 (m; 1 H); 8.39 (d; J = 7.96 Hz; 2 H); | |
| | | | 7.87 (s; 1 H); 7.80-7.83 (m; 1 H); 7.62-7.68 (m; 2 | |
| | | | H); 7.46-7.57 (m; 3 H); 7.23 (d; J = 7.07 Hz; 1 H); | |
| | | | 3.55-3.66 (m; 6 H), 2.43 (m; 4 H). | |
| 40 | A | 536.2 | ¹H-NMR (DMSO-d₆, 300 MHz): δ 13.23 (br s; 1 | 18 |
| | | | H); 8.50-8.52 (m; 1 H); 8.39 (s; 1 H); 8.29 (d; J = | |
| | | | 7.85 Hz; 2 H); 7.84 (s; 1 H); 7.78 (d; J = 3.65 Hz; | |
| | | | 1 H); 7.56-7.61 (m; 1 H); 7.52-7.55 (m; 1 H); | |
| | | | 7.43-7.50 (m; 1 H); 7.21 (d; J = 7.36 Hz; 1 H); | |
| | | | 3.57-3.60 (m; 6 H); 2.43 (m; 4 H). | |
| 41 | A | 439 | ¹H-NMR (DMSO-d₆, 300 MHz): δ 9.54 (d; J = | 10 |
| | | | 5.18 Hz; 1 H); 8.88 (d; J = 8.65 Hz; 1 H); 8.59 (s; | |
| | | | 1 H); 8.12 (dd; J = 8.67; 4.98 Hz; 1 H); 7.88-7.90 | |
| | | | (m; 2 H); 7.74 (d; J = 3.41 Hz; 1 H); 7.66 (d; J = | |
| | | | 8.43 Hz; 1 H); 7.52 (t; J = 7.72 Hz; 1 H); 7.25 (s; 2 H). | |
| 42 | A | 444 | ¹H-NMR (DMSO-d₆, 400 MHz): δ 13.26 (s; 1 H); | 11 |
| | | | 8.52 (dd; J = 3.19; 2.22 Hz; 1 H); 8.14-8.18 (m; 2 | |
| | | | H); 7.86 (s; 1 H); 7.81-7.83 (m; 1 H); 7.67-7.70 | |
| | | | (m; 1 H); 7.60-7.63 (m; 1 H); 7.49-7.53 (m; 1 H); | |
| | | | 7.23 (d; J = 6.92 Hz; 1 H). | |
| 43 | A | 405.1 | ¹H-NMR (DMSO-d₆, 400 MHz): δ 13.25 (s; 0 H); | 20 |
| | | | 12.36 (s; 1 H); 8.51 (s; 1 H); 7.83 (s; 1 H); 7.77 | |
| | | | (d; J = 3.25 Hz; 1 H); 7.59-7.62 (m; 2 H); 7.49 (t; | |
| | | | J = 7.71 Hz; 1 H); 7.20 (d; J = 6.92 Hz; 1 H); 4.46 | |
| | | | (s; 2 H); 3.46 (s; 3 H). | |
| 44 | B | 546.1 | 1H NMR (400 MHz, DMSO): ? 13.23 (s; 1 H); | 11 |
| | | | 12.31 (s; 1 H); 8.97 (d; J = 2.45 Hz; 1 H); 8.33 | |
| | | | (ddd; J = 9.09; 2.45; 0.86 Hz; 1 H); 7.82 (s; 1 H); | |
| | | | 7.64 (s; 1 H); 7.58 (d; J = 8.45 Hz; 1 H); 7.45 (dd; | |
| | | | J = 8.39; 6.95 Hz; 1 H); 7.14 (d; J = 6.99 Hz; 1 | |
| | | | H); 6.99 (d; J = 9.23 Hz; 1 H); 4.00-4.06 (m; 2 H); | |
| | | | 3.38-3.50 (m; 6 H); 1.43-1.57 (m; 4 H); 1.15 (s; 3 H). | |
| 45 | B | 531.8 | 1H NMR (400 MHz, DMSO): δ 9.01 (d; J = 2.37 | 18 |
| | | | Hz; 1 H); 8.37 (d; J = 9.14 Hz; 1 H); 7.81 (s; 1 H); | |
| | | | 7.53-7.55 (m; 2 H); 7.43 (t; J = 7.66 Hz; 1 H); | |
| | | | 7.12 (d; J = 6.93 Hz; 1 H); 6.93 (d; J = 9.12 Hz; 1 | |
| | | | H); 4.11 (d; J = 12.97 Hz; 2 H); 3.73 (s; 1 H); | |
| | | | 3.20 (d; J = 11.25 Hz; 3 H) 1.79 (m; 2 H); 1.36 (d; | |
| | | | J = 11.14 Hz; 2 H). | |
| 46 | A | 451.2 | ¹H NMR (DMSO-d₆, 400 MHz): δ 13.22 (s; 1 H); | >2000 |
| | | | 12.55 (s; 1 H); 8.47 (s; 1 H); 7.79-7.79 (m; 1 H); | |
| | | | 7.73 (d; J = 3.29 Hz; 1 H); 7.57-7.60 (m; 1 H); | |
| | | | 7.51-7.53 (m; 3 H); 7.47 (t; J = 7.63 Hz; 1 H); | |
| | | | 7.35 (t; J = 7.50 Hz; 2 H); 7.24-7.28 (m; 1 H); | |
| | | | 7.16 (d; J = 0.88 Hz; 1 H); 4.03 (s; 2 H). | |
| 47 | B | 546.1 | 1H NMR (400 MHz, DMSO): ? 13.23 (s; 1 H); | 11 |
| | | | 12.31 (s; 1 H); 8.97 (d; J = 2.45 Hz; 1 H); 8.33 | |
| | | | (ddd; J = 9.09; 2.45; 0.86 Hz; 1 H); 7.82 (s; 1 H); | |
| | | | 7.64 (s; 1 H); 7.58 (d; J = 8.45 Hz; 1 H); 7.45 (dd; | |
| | | | J = 8.39; 6.95 Hz; 1 H); 7.14 (d; J = 6.99 Hz; 1 | |
| | | | H); 6.99 (d; J = 9.23 Hz; 1 H); 4.00-4.06 (m; 2 H); | |
| | | | 3.38-3.50 (m; 6 H); 1.43-1.57 (m; 4 H); 1.15 (s; 3 H). | |
| 48 | C | 372.2 | ¹H NMR (400 MHz, DMSO-d6) δ 13.22 (bs, 1H), | 30 |
| | | | 12.19 (bs, 1H), 8.02 (s, 1H), 7.90 (s, 1H), 7.61- | |
| | | | 7.57 (m, 3H), 7.45-7.49 (m, 2H), 7.17-7.23 (m, 3H). | |
| 49 | C | 422.3 | ¹H NMR (400 MHz, DMSO d₆) δ 13.25 (s, 1H), | 990 |
| | | | 12.14 (bs, 1H), 8.42 (bs, 1H), 8.03 (s, 1H), 7.92 | |
| | | | (s, 2H), 7.86 (s, 1H), 7.61(d, J = 7.6 Hz, 3H), | |
| | | | 7.54 (s, 1H), 7.48 (t, J = 8 Hz, 1H), 7.23(d, J = | |
| | | | 6.8 Hz, 1H). | |
| 50 | B | 561.4 | 1H NMR (400 MHz, DMSO-d6) δ 13.22 (s, 1H), | 2 |
| | | | 12.64 (s, 1H), 8.76 (d, J = 5.0 Hz, 2H), 8.15 (s, | |
| | | | 1H), 8.13-8.06 (m, 2H), 7.95 (d, J = 7.5 Hz, | |
| | | | 1H), 7.84 (s, 1H), 7.74 (t, J = 7.9 Hz, 1H), 7.57 | |
| | | | (d, J = 8.4 Hz, 1H), 7.45 (m, 1H), 7.21 (m, 1H), | |
| | | | 6.63 (d, J = 8.1 Hz, 1H), 4.32 (m, 2H), 3.36 (m, | |
| | | | 4H), 2.56 (m, 2H), 2.24 (m, 4H) | |
| 51 | D | 357.2 | ¹H NMR (400 MHz, DMSO-d₆) δ 13.03 (s, 1H), | 1230 |
| | | | 9.43 (dd, J = 2.6, 1.2 Hz, 1H), 9.11 (dd, J = 5.6, | |
| | | | 1.2 Hz, 1H), 8.56 (s, 1H), 7.79 (dd, J = 5.6, 2.6 | |
| | | | Hz, 1H), 7.54 (d, J = 3.7 Hz, 2H), 7.47 (dd, J = | |
| | | | 8.4, 1.1 Hz, 1H), 7.31 (d, J = 8.6 Hz, 1H), 5.97 (s, | |
| | | | 2H), 4.66 (t, J = 1.3 Hz, 2H), 2.19 (s, 3H). | |
| 52 | B | 560.4 | 1H NMR (400 MHz, DMSO-d6) δ 13.21 (s, 1H), | 5 |
| | | | 12.58 (s, 1H), 8.72 (d, J = 5.2 Hz, 2H), 8.07- | |
| | | | 7.98 (m, 2H), 7.86 (s, 1H), 7.62-7.51 (m, 2H), | |
| | | | 7.45 (m, 1H), 7.28 (t, J = 7.9 Hz, 1H), 7.25-7.15 | |
| | | | (m, 3H), 6.88-6.80 (m, 1H), 4.07 (m, 2H), 3.50 | |
| | | | (m, 4H), 2.65 (m, 2H), 2.40 (m, 4H). | |
| 53 | B | 460.2 | 1H NMR (400 MHz, DMSO-d6) δ 13.19 (s, 1H), | 4 |
| | | | 12.56 (s, 1H) 9.32 (d, J = 2.2 Hz, 1H), 8.72 (d, J = | |
| | | | 4.7 Hz, 1H), 8.50 (m, 1H), 8.25 (s, 1H), 7.99 (s, | |
| | | | 1H), 7.80 (s, 1H), 7.69 (s, 1H), 7.63-7.53 (m, | |
| | | | 2H), 7.45 (m, 1H), 7.16 (d, J = 7.0 Hz, 1H), 6.90 | |
| | | | (s, 2H), 3.69 (m, 1H), 1.06-0.96 (m, 2H), 0.99- | |
| | | | 0.85 (m, 2H). | |
| 54 | B | 545.2 | 1H NMR (400 MHz, DMSO-d6) δ 13.21 (s, 2H), | 2 |
| | | | 8.76 (d, J = 5.6 Hz, 2H), 8.17 (s, 1H), 8.14 (s, | |
| | | | 1H), 8.12-8.06 (m, 2H), 7.93 (d, J = 7.5 Hz, | |
| | | | 1H), 7.84 (s, 1H), 7.74 (t, J = 7.9 Hz, 1H), 7.57 | |
| | | | (d, J = 8.4 Hz, 1H), 7.45 (m, 1H), 7.20 (d, J = 6.8 | |
| | | | Hz, 1H), 6.64 (d, J = 8.0 Hz, 1H), 4.30 (t, J = 6.3 | |
| | | | Hz, 2H), 2.69 (t, J = 6.3 Hz, 2H), 2.31 (m, 4H), | |
| | | | 1.49 (m, 4H). | |
| 55 | B | 532.4 | 1H NMR (400 MHz, DMSO-d6) δ 13.23 (s, 1H), | 4 |
| | | | 12.56 (bs, 1H), 8.88-8.66 (m, 2H), 8.12 (s, 1H), | |
| | | | 8.11-8.05 (m, 2H), 7.92 (d, J = 7.5 Hz, 1H), | |
| | | | 7.87 (s, 1H), 7.75 (t, J = 7.8 Hz, 1H), 7.57 (d, J = | |
| | | | 8.4 Hz, 1H), 7.45 (dd, J = 8.4, 7.0 Hz, 1H), 7.23 | |
| | | | (d, J = 6.9 Hz, 1H), 6.65 (d, J = 8.1 Hz, 1H), 4.37- | |
| | | | 4.05 (m, 3H), 3.78-3.61 (m, 1H), 3.54 (q, J = | |
| | | | 7.3 Hz, 1H), 1.96-1.60 (m, 3H), 1.54 (m, 1H) | |
| 56 | B | 450.2 | 1H NMR (400 MHz, DMSO-d6) δ 13.20 (s, 1H), | 7 |
| | | | 12.56 (bs, 1H), 8.82-8.76 (m, 2H), 8.10-8.02 | |
| | | | (m, 2H), 7.82 (s, 1H), 7.57-7.52 (m, 1H), 7.43 | |
| | | | (dd, J = 8.4, 7.0 Hz, 1H), 7.32 (s, 1H), 7.16 (d, J = | |
| | | | 7.0 Hz, 1H), 6.92 (s, 2H), 5.73 (m, 1H), 3.07 | |
| | | | (m, 2H), 2.73 (m, @H), 2.67 (m, 2H), 2.32 (s, 3H). | |
| 57 | B | 478.2 | 1H NMR (400 MHz, DMSO-d6) δ 13.20 (s, 1H), | 2 |
| | | | 12.53 (s, 1H), 8.77 (d, J = 5.0 Hz, 2H), 8.14- | |
| | | | 8.00 (m, 2H), 7.83 (s, 1H), 7.55 (d, J = 8.3 Hz, | |
| | | | 1H), 7.43 (t, J = 7.7 Hz, 1H), 7.36 (d, J = 5.8 Hz, | |
| | | | 1H), 7.16 (m, 1H), 6.91 (s, 2H), 5.79 (m, 1H), | |
| | | | 4.06 (m, 2H), 3.65 (m, 2H), 2.73 (m, 1H), 2.64 | |
| | | | (m, 1H), 2.05 (s, 2H), 2.00 (s, 1H). | |
| 58 | B | 477.3 | 1H NMR (400 MHz, DMSO-d6) δ 13.21 (s, 1H), | 1 |
| | | | 12.58 (s, 1H), 8.79 (d, J = 5.2 Hz, 2H), 8.42 (s, | |
| | | | 1H), 8.17 (s, 1H), 8.14-8.07 (m, 2H), 7.81 (s, | |
| | | | 1H), 7.77 (s, 1H), 7.57 (d, J = 8.4 Hz, 1H), 7.51- | |
| | | | 7.35 (m, 1H), 7.17 (d, J = 6.9 Hz, 1H), 6.90 (s, | |
| | | | 2H), 5.58 (m, 1H), 4.90 (d, J = 7.0 Hz, 4H). | |
| 59 | B | 394.3 | 1H NMR (400 MHz, DMSO-d6) δ 13.18 (s, 1H), | 5 |
| | | | 12.47 (s, 1H), 8.75 (d, J = 5.0 Hz, 2H), 8.16 (d, J = | |
| | | | 5.1 Hz, 2H), 7.74 (s, 1H), 7.53 (d, J = 8.4 Hz, | |
| | | | 1H), 7.42 (t, J = 7.8 Hz, 1H), 7.11 (d, J = 7.1 Hz, | |
| | | | 1H), 6.95 (s, 1H), 6.70 (s, 2H), 2.82 (s, 1H), 0.91 | |
| | | | (m, 2H), 0.64 (m, 2H). | |
| 60 | B | 518.2 | 1H NMR (400 MHz, DMSO-d6) δ 13.20 (s, 1H), | 3 |
| | | | 12.56 (bs, 1H), 8.87-8.73 (m, 2H), 8.28 (s, 1H), | |
| | | | 8.12-8.07 (m, 2H), 8.03 (s, 1H), 7.80 (s, 1H), | |
| | | | 7.75 (s, 1H), 7.57 (d, J = 8.4 Hz, 1H), 7.45 (dd, J = | |
| | | | 8.4, 7.0 Hz, 1H), 7.16 (d, J = 6.7 Hz, 1H), 6.86 | |
| | | | (bs, 2H), 4.09 (m, 1H), 2.84 (m, 2H), 2.18 (s, 3H), | |
| | | | 2.11-1.91 (m, 6H). | |
| 61 | B | 435.2 | 1H NMR (400 MHz, DMSO-d6) δ 13.22 (s, 1H), | 2 |
| | | | 12.56 (s, 1H), 8.79 (d, J = 5.2 Hz, 2H), 8.12- | |
| | | | 8.10 (m, 2H), 8.09 (s, 1H), 7.78 (s, 1H), 7.68 (d, J = | |
| | | | 2.1 Hz, 1H), 7.58 (d, J = 8.3 Hz, 1H), 7.50- | |
| | | | 7.42 (m, 1H), 7.16 (d, J = 6.9 Hz, 1H), 7.08 (d, J = | |
| | | | 2.2 Hz, 1H), 6.98 (bs, 2H), 3.80 (s, 3H). | |
| 62 | D | 374.2 | ¹H NMR (400 MHz, DMSO-d₆) δ 13.03 (s, 1H), | 539 |
| | | | 8.57 (d, J = 2.4 Hz, 1H), 8.45 (s, 1H), 8.40 | |
| | | | (dd, J = 4.9, 1.1 Hz, 1H), 7.62 (dd, J = 6.9, 5.0 | |
| | | | Hz, 1H), 7.53 (t, J = 1.3 Hz, 1H), 7.50-7.43 (m, | |
| | | | 1H), 7.30 (d, J = 8.6 Hz, 1H), 7.26 (d, J = 1.9 Hz, | |
| | | | 1H), 5.88 (s, 2H), 4.65-4.16 (m, 2H), 2.19 (s, 3H). | |
| 63 | D | 362.2 | ¹H NMR (400 MHz, DMSO-d₆) δ 13.05 (s, 1H), | 16 |
| | | | 8.55 (s, 1H), 7.83 (d, J = 3.3 Hz, 1H), 7.63 (s, | |
| | | | 1H), 7.61 (d, J = 3.3 Hz, 1H), 7.52 (s, 1H), 7.50- | |
| | | | 7.46 (m, 1H), 7.32 (d, J = 8.6 Hz, 1H), 6.00 (s, | |
| | | | 2H), 4.63 (s, 2H), 2.18 (s, 3H). | |
| 64 | B | 437.3 | 1H NMR (400 MHz, DMSO-d6) δ 13.20 (s, 1H), | 3 |
| | | | 12.53 (s, 1H), 8.77 (d, J = 5.5 Hz, 2H), 8.14- | |
| | | | 8.02 (m, 2H), 7.83 (s, 1H), 7.55 (d, J = 8.4 Hz, | |
| | | | 1H), 7.43 (m, 1H), 7.36 (s, 1H), 7.16 (d, J = 6.9 | |
| | | | Hz, 1H), 6.92 (s, 2H), 5.85 (m, 1H), 4.19 (m, 2H), | |
| | | | 3.81 (m, 2H), 2.66 (m, 2H). | |
| 65 | B | 532.4 | 1H NMR (400 MHz, DMSO-d6) δ 13.25 (s, 1H), | 4 |
| | | | 12.64 (s, 1H), 8.77 (d, J = 5.4 Hz, 2H), 8.18 (s, | |
| | | | 1H), 8.11-8.02 (m, 2H), 7.94 (d, J = 7.5 Hz, | |
| | | | 1H), 7.90 (s, 1H), 7.74 (t, J = 7.9 Hz, 1H), 7.57 | |
| | | | (d, J = 8.4 Hz, 1H), 7.46 (m, 1H), 7.26 (d, J = 6.8 | |
| | | | Hz, 1H), 6.63 (dd, J = 8.1, 0.7 Hz, 1H), 5.16 (m, | |
| | | | 1H), 3.73 (m, 2H), 3.30 (m, 2H), 1.95 (m, 2H), | |
| | | | 1.65-1.44 (m, 2H). | |
| 66 | B | 520.2 | 1H NMR (400 MHz, DMSO-d6) δ 13.22 (s, 1H), | 3 |
| | | | 12.65 (s, 1H), 8.94-8.71 (m, 2H), 8.13 (s, 1H), | |
| | | | 8.11-8.01 (m, 2H), 7.92 (d, J = 7.6 Hz, 1H), | |
| | | | 7.86 (s, 1H), 7.75 (t, J = 7.8 Hz, 1H), 7.60-7.54 | |
| | | | (m, 1H), 7.45 (dd, J = 8.4, 7.0 Hz, 1H), 7.21 (d, J = | |
| | | | 7.0 Hz, 1H), 6.66 (d, J = 8.1 Hz, 1H), 4.32- | |
| | | | 4.01 (m, 2H), 3.60 (m, 2H), 3.14 (s, 3H), 1.02 (d, | |
| | | | J = 6.3 Hz, 3H). | |
| 67 | B | 505.3 | 1H NMR (400 MHz, DMSO-d6) δ 13.20 (s, 1H), | 3 |
| | | | 12.56 (s, 1H), 8.90-8.73 (m, 2H), 8.31 (s, 1H), | |
| | | | 8.13-8.08 (m, 2H), 8.05 (s, 1H), 7.80 (s, 1H), | |
| | | | 7.75 (s, 1H), 7.57 (d, J = 8.4 Hz, 1H), 7.45 (dd, J = | |
| | | | 8.4, 7.0 Hz, 1H), 7.17 (d, J = 6.8 Hz, 1H), 6.85 | |
| | | | (s, 2H), 4.37 (m, 1H), 3.99-3.89 (m, 2H), 3.44 | |
| | | | (m, 2H), 2.08-1.86 (m, 4H). | |
| 68 | B | 475.3 | 1H NMR (400 MHz, DMSO-d6) δ 13.20 (s, 1H), | 6 |
| | | | 12.45 (bs, 1H), 8.85-8.73 (m, 2H), 8.33 (s, 1H), | |
| | | | 8.16-8.10 (m, 2H), 7.95 (s, 1H), 7.81 (s, 1H), | |
| | | | 7.72 (s, 1H), 7.56 (d, J = 8.4 Hz, 1H), 7.45 (dd, J = | |
| | | | 8.4, 7.0 Hz, 1H), 7.17 (d, J = 6.8 Hz, 1H), 6.88 | |
| | | | (s, 2H), 3.97 (d, J = 7.1 Hz, 2H), 1.23 (m, 1H), | |
| | | | 0.56-0.48 (m, 2H), 0.41-0.34 (m, 2H). | |
| 69 | D | 335.0 | ¹H NMR (400 MHz, DMSO-d₆) δ 9.31 (br s, 1H), | 864 |
| | | | 8.51 (br s, 1H), 7.14 (s, 1H), 7.11 (dd, J = 8.3, | |
| | | | 0.8 Hz, 1H), 6.72 (dd, J = 8.2, 2.6 Hz, 1H), 6.53 | |
| | | | (d, J = 2.6 Hz, 1H), 5.58 (br s, 2H), 4.24-4.11 | |
| | | | (m, 2H), 1.97 (s, 3H). | |
| 70 | B | 532.4 | 1H NMR (400 MHz, DMSO-d6) δ 13.22 (s, 1H), | 4 |
| | | | 12.55 (bs, 1H), 8.75 (d, J = 5.3 Hz, 2H), 8.12 (s, | |
| | | | 1H), 8.11-8.06 (m, 2H), 7.93 (d, J = 7.6 Hz, | |
| | | | 1H), 7.87 (s, 1H), 7.75 (t, J = 7.8 Hz, 1H), 7.56 | |
| | | | (d, J = 8.3 Hz, 1H), 7.45 (dd, J = 8.4, 6.9 Hz, 1H), | |
| | | | 7.23 (d, J = 6.9 Hz, 1H), 6.65 (d, J = 8.1 Hz, 1H), | |
| | | | 4.23 (m, 1H), 4.18-4.04 (m, 2H), 3.66 (dt, J = | |
| | | | 8.2, 6.6 Hz, 1H), 3.60-3.50 (m, 1H), 1.87-1.62 | |
| | | | (m, 3H), 1.54 (m, 1H). | |
| 71 | B | 471.3 | 1H NMR (400 MHz, DMSO-d6) δ 13.20 (s, 1H), | 21 |
| | | | 12.52 (s, 1H), 8.90-8.56 (m, 2H), 8.16-8.02 | |
| | | | (m, 2H), 7.83 (s, 1H), 7.55 (d, J = 8.4 Hz, 1H), | |
| | | | 7.43 (dd, J = 8.4, 7.0 Hz, 1H), 7.31 (s, 1H), 7.16 | |
| | | | (d, J = 6.7 Hz, 1H), 6.96 (s, 2H), 5.60 (s, 1H), | |
| | | | 2.83 (m, 2H), 2.70 (m, 2H), 2.16 (m, 2H). | |
| 72 | B | 461.2 | 1H NMR (400 MHz, DMSO-d6) δ 13.20 (s, 1H), | 3 |
| | | | 12.56 (s, 1H), 8.87-8.70 (m, 2H), 8.25 (s, 1H), | |
| | | | 8.14-8.07 (m, 2H), 8.00 (s, 1H), 7.80 (s, 1H), | |
| | | | 7.73 (s, 1H), 7.57 (d, J = 8.4 Hz, 1H), 7.45 (dd, J = | |
| | | | 8.4, 7.0 Hz, 1H), 7.16 (d, J = 6.9 Hz, 1H), 7.06- | |
| | | | 6.58 (m, 2H), 3.71 (m, 1H), 1.04 (m, 2H), 0.93 | |
| | | | (m, 2H). | |
| 73 | B | 471.2 | 1H NMR (400 MHz, DMSO-d6) δ 13.20 (s, 1H), | 4 |
| | | | 12.56 (bs, 1H), 8.84-8.76 (m, 2H), 8.71 (s, 1H), | |
| | | | 8.36 (s, 1H), 8.12-8.04 (m, 2H), 7.82 (m, 3H), | |
| | | | 7.57 (d, J = 8.4 Hz, 1H), 7.50-7.39 (m, 1H), | |
| | | | 7.18 (d, J = 7.0 Hz, 1H). | |
| 74 | B | 461.3 | 1H NMR (400 MHz, DMSO-d6) δ 13.25-13.17 | 11 |
| | | | (s, 1H), 12.67 (s, 1H), 9.24 (s, 1H), 8.72 (d, J = | |
| | | | 4.7 Hz, 1H), 8.43 (d, J = 8.1 Hz, 1H), 8.29 (d, J = | |
| | | | 2.0 Hz, 1H), 7.82 (s, 1H), 7.72 (s, 1H), 7.57 (m, | |
| | | | 2H), 7.46 (t, J = 7.8 Hz, 1H), 7.18 (m, 3H), 6.16 | |
| | | | (d, J = 1.8 Hz, 1H), 1.95-1.82 (m, 1H), 0.84- | |
| | | | 0.74 (m, 2H), 0.70-0.54 (m, 2H). | |
| 75 | B | 439.1 | 1H NMR (400 MHz, DMSO-d6) δ 13.23 (s, 1H), | 6 |
| | | | 12.56 (bs, 1H), 9.25 (d, J = 2.2 Hz, 1H), 8.71 (d, | |
| | | | J = 4.9 Hz, 1H), 8.52 (d, J = 4.5 Hz, 1H), 8.48- | |
| | | | 8.39 (m, 1H), 7.83 (s, 1H), 7.68 (dd, J = 6.1, 2.6 | |
| | | | Hz, 2H), 7.56 (t, J = 6.9 Hz, 2H), 7.45 (dd, J = | |
| | | | 8.4, 7.0 Hz, 1H), 7.18 (m, 2H). | |
| 76 | B | 505.3 | 1H NMR (400 MHz, DMSO-d6) δ 13.22 (s, 1H), | 8 |
| | | | 12.56 (bs, 1H), 9.26 (s, 1H), 8.70 (d, J = 4.8 Hz, | |
| | | | 1H), 8.45 (d, J = 7.8 Hz, 1H), 8.39 (d, J = 2.4 Hz, | |
| | | | 1H), 7.82 (s, 1H), 7.72 (s, 1H), 7.62-7.51 (m, | |
| | | | 2H), 7.45 (t, J = 7.7 Hz, 1H), 7.17 (d, J = 7.0 Hz, | |
| | | | 3H), 6.34 (d, J = 2.4 Hz, 1H), 4.04-3.78 (m, | |
| | | | 2H), 3.45 (m, 2H), 1.79 (m, 2H), 1.63 (m, 2H). | |
| 77 | B | 422.3 | 1H NMR (400 MHz, DMSO-d6) δ 13.24 (s, 1H), | 4 |
| | | | 12.72 (s, 1H), 8.84-8.66 (m, 2H), 8.41 (d, J = | |
| | | | 2.3 Hz, 1H), 8.05-7.98 (m, 2H), 7.83 (s, 1H), | |
| | | | 7.75 (s, 1H), 7.66-7.55 (m, 2H), 7.46 (dd, J = | |
| | | | 8.4, 7.0 Hz, 1H), 7.19 (m, 3H), 6.49 (s, 1H). | |
| 78 | B | 436.3 | H NMR (400 MHz, DMSO-d6) δ 13.20 (s, 1H), | 7 |
| | | | 12.52 (s, 1H), 8.95-8.73 (m, 2H), 8.15-7.96 | |
| | | | (m, 2H), 7.81 (s, 1H), 7.55 (d, J = 8.4 Hz, 1H), | |
| | | | 7.44 (dd, J = 8.4, 6.9 Hz, 1H), 7.29 (s, 1H), 7.16 | |
| | | | (d, J = 6.9 Hz, 1H), 6.91 (s, 2H), 6.03 (d, J = 4.6 | |
| | | | Hz, 1H), 4.54 (m, 1H), 4.47 (m, 1H), 2.97 (m, | |
| | | | 1H), 2.51 (m, 1H), 2.05 (m, 2H), 1.95-1.77 (m, 2H). | |
| 79 | B | 421.3 | 1H NMR (400 MHz, DMSO-d6) δ 13.23 (s, 1H), | 3 |
| | | | 12.69 (s, 1H), 9.23 (s, 1H), 8.71 (d, J = 4.7 Hz, | |
| | | | 1H), 8.50-8.38 (m, 2H), 7.83 (s, 1H), 7.73 (s, | |
| | | | 1H), 7.67-7.54 (m, 3H), 7.50-7.42 (m, 1H), | |
| | | | 7.19 (d, J = 6.9 Hz, 3H), 6.48 (t, J = 2.2 Hz, 1H). | |
| 80 | B | 448.3 | 1H NMR (400 MHz, DMSO-d6) δ 13.23 (s, 1H), | 6 |
| | | | 12.51 (s, 1H), 9.29 (s, 1H), 8.75 (d, J = 4.9 Hz, | |
| | | | 1H), 8.48 (m, 1H), 8.11 (s, 1H), 7.87 (s, 1H), 7.76 | |
| | | | (s, 1H), 7.57 (m, 2H), 7.52-7.36 (m, 2H), 7.20 | |
| | | | (d, J = 6.9 Hz, 1H), 6.52 (s, 1H), 6.18 (d, J = 9.0 | |
| | | | Hz, 1H). | |
| 81 | B | 505.2 | 1H NMR (400 MHz, DMSO-d6) δ 13.20 (s, 1H), | 4 |
| | | | 12.53 (s, 1H), 9.32 (s, 1H), 8.74 (d, J = 4.5 Hz, | |
| | | | 1H), 8.50 (m, 1H), 8.30 (s, 1H), 8.03 (s, 1H), 7.81 | |
| | | | (s, 1H), 7.74 (s, 1H), 7.66-7.54 (m, 2H), 7.45 | |
| | | | (dd, J = 8.4, 7.0 Hz, 1H), 7.27-7.13 (m, 1H), | |
| | | | 6.69 (bs, 2H), 4.37 (m, 1H), 3.97-3.88 (m, 2H), | |
| | | | 3.50-3.39 (m, 2H), 2.07-1.86 (m, 4H). | |
| 82 | C | 355.2 | ¹H NMR (400 MHz, DMSO-d₆) δ 13.19 (s, 1H), | 35 |
| | | | 12.14 (s, 1H), 8.51 (s, 1H), 8.27-8.06 (m, 1H), | |
| | | | 8.03 (s, 1H), 7.97 (s, 1H), 7.79 (s, 1H), 7.72 | |
| | | | (t, J = 7.8 Hz, 1H), 7.55 (d, J = 8.4 Hz, 1H), 7.44 | |
| | | | (m, 1H), 7.17 (m, 3H). | |
| 83 | B | 433.2 | 1H NMR (400 MHz, DMSO-d6) δ 13.25 (s, 2H), | 5 |
| | | | 12.81 (s, 1H), 8.89 (s, 2H), 8.30 (d, J = 5.7 Hz, | |
| | | | 2H), 7.80 (d, J = 0.9 Hz, 1H), 7.71 (s, 1H), 7.65- | |
| | | | 7.56 (m, 1H), 7.45 (dd, J = 8.4, 7.0 Hz, 1H), 7.13 | |
| | | | (dd, J = 7.0, 0.7 Hz, 1H). | |
| 84 | C | 359.2 | ¹H NMR (400 MHz, DMSO-d₆) δ 13.19 (s, 1H), | 44 |
| | | | 9.49 (bs, 1H), 9.10 (m, 1H), 8.05 (s, 1H), 7.96- | |
| | | | 7.82 (m, 2H), 7.66 (s, 1H), 7.56 (d, J = 8.4 Hz, | |
| | | | 1H), 7.44 (m, 1H), 7.19 (m, 2H). | |
| 85 | D | 255.1 | ¹H NMR (400 MHz, DMSO-d₆) δ 9.25 (s, 1H), | 16000 |
| | | | 8.24 (s, 1H), 7.09 (dd, J = 8.2, 0.8 Hz, 1H), 7.01 | |
| | | | (d, J = 7.5 Hz, 1H), 6.73-6.66 (m, 2H), 6.52 | |
| | | | (d, J = 2.6 Hz, 1H), 5.42 (br s, 2H), 4.28 (s, 2H), | |
| | | | 1.96 (s, 3H). | |
| 86 | B | 461.1 | 1H NMR (400 MHz, DMSO-d6) δ 13.24 (s, 1H), | 18 |
| | | | 12.50 (bs, 1H), 8.77 (d, J = 5.7 Hz, 2H), 8.08- | |
| | | | 7.96 (m, 3H), 7.83 (s, 1H), 7.74 (s, 1H), 7.58 (d, J = | |
| | | | 8.4 Hz, 1H), 7.46 (dd, J = 8.4, 7.0 Hz, 1H), 7.18 | |
| | | | (m, 3H), 2.68 (t, J = 7.2 Hz, 2H), 2.61 (d, J = 7.5 | |
| | | | Hz, 2H), 2.35 (m, 2H). | |
| 87 | B | 489.3 | 1H NMR (400 MHz, DMSO-d6) δ 13.24 (s, 1H), | 100 |
| | | | 12.80 (bs, 1H), 8.96 (s, 1H), 8.73 (d, J = 5.5 Hz, | |
| | | | 2H), 8.08 (s, 1H), 7.99 (d, J = 5.6 Hz, 2H), 7.86 | |
| | | | (s, 1H), 7.73 (s, 1H), 7.59 (d, J = 8.5 Hz, 1H), | |
| | | | 7.53-7.41 (m, 1H), 7.20 (d, J = 7.0 Hz, 1H), | |
| | | | 6.91 (s, 2H), | |
| 88 | C | 498.2 | 1H NMR (400 MHz, DMSO-d6) δ 13.03 (s, 1H), | 5 |
| | | | 8.05 (s, 1H) 12.10 (bs, 1H), , 7.97 (s, 1H), 7.89 | |
| | | | (d, J = 7.6 Hz, 1H), 7.64 (t, J = 7.9 Hz, 1H), 7.52 | |
| | | | (s, 1H), 7.47 (d, J = 8.5 Hz, 1H), 7.32 (d, J = 8.6 | |
| | | | Hz, 1H), 6.57 (d, J = 8.1 Hz, 1H), 4.27 (t, J = 6.4 | |
| | | | Hz, 2H),3, 30 (m, 4H), 2.56-2.48 (m, 2H), 2.20 | |
| | | | (m, 7H). | |
| 89 | C | 482.2 | ¹H NMR (400 MHz, DMSO-d₆) δ 13.03 (s, 1H), | 13 |
| | | | 12.15 (s, 1H), 8.17 (s, 1H), 8.05 (s, 1H), 7.98 (s, | |
| | | | 1H), 7.88 (d, J = 7.6 Hz, 1H), 7.63 (t, J = 7.9 Hz, | |
| | | | 1H), 7.51 (d, J = 1.0 Hz, 1H), 7.47 (d, J = 8.5 Hz, | |
| | | | 1H), 7.32 (d, J = 8.5 Hz, 1H), 6.57 (d, J = 8.1 Hz, | |
| | | | 1H), 4.26 (m, 2H), 3.17-3.09 (m, 2H), 2.65 | |
| | | | (t, J = 6.4 Hz, 2H), 2.28 (m, 4H), 2.19 (s, 3H), | |
| | | | 1.47 (m, 2H). | |
| 90 | C | 479.2 | 1H NMR (400 MHz, DMSO-d6) δ 13.03 (s, 1H), | 11 |
| | | | 12.15 (s, 1H), 8.04 (s, 1H), 7.86 (m, 2H), 7.67 | |
| | | | (m, 1H), 7.57-7.43 (m, 2H), 7.33 (d, J = 8.5 Hz, | |
| | | | 1H), 6.83 (s, 1H), 6.65 (d, J = 8.2 Hz, 2H), 5.21 | |
| | | | (s, 2H), 2.20 (s, 3H). | |
| 91 | C | 466.2 | | 5 |
| 92 | B | 395.2 | 1H NMR (400 MHz, DMSO-d6) δ 13.18 (s, 1H), | 7 |
| | | | 12.44 (s, 1H), 9.35 (s, 1H), 8.72 (d, J = 4.2 Hz, | |
| | | | 1H), 8.53 (dt, J = 8.1, 2.0 Hz, 1H), 7.74 (s, 1H), | |
| | | | 7.66-7.52 (m, 2H), 7.42 (dd, J = 8.4, 7.0 Hz, | |
| | | | 1H), 7.11 (d, J = 6.9 Hz, 1H), 6.95 (s, 1H), 6.60 | |
| | | | (bs, 1H), 2.80 (m, 1H), 0.93-0.86 (m, 2H), 0.63 | |
| | | | (m, 2H). | |
| 93 | D | 338.1 | ¹H NMR (400 MHz, DMSO-d₆) δ 9.33 (s, 1H), | 22 |
| | | | 8.55 (s, 1H), 7.87 (d, J = 3.3 Hz, 1H), 7.65 (d, J = | |
| | | | 3.3 Hz, 1H), 7.54 (s, 1H), 7.14 (d, J = 8.6 Hz, | |
| | | | 1H), 6.75 (dd, J = 8.2, 2.6 Hz, 1H), 6.60 (d, J = | |
| | | | 2.6 Hz, 1H), 6.02 (br s, 2H), 4.61 (s, 2H), 2.54 (s, | |
| | | | 6H), 2.01 (s, 3H). | |
| 94 | C | 428.2 | 1H NMR (400 MHz, DMSO-d6) δ 13.18 (s, 1H), | 46 |
| | | | 12.07 (s, 1H), 7.94 (s, 1H), 7.83 (s, 1H), 7.54 (d, | |
| | | | J = 8.4 Hz, 1H), 7.46-7.35 (m, 2H), 7.27-7.12 | |
| | | | (m, 2H), 7.11-7.00 (m, 2H), 6.74 (dd, J = 8.2, | |
| | | | 2.5 Hz, 1H), 4.57 (s, 2H). | |
| 95 | C | 451.2 | 1H NMR (400 MHz, DMSO-d6) δ 13.18 (s, 1H), | 10 |
| | | | 12.07 (s, 1H), 8.11 (d, J = 0.8 Hz, 1H), 7.94 (s, | |
| | | | 1H), 7.84 (s, 1H), 7.54 (d, J = 8.4 Hz, 1H), 7.48- | |
| | | | 7.35 (m, 2H), 7.28-7.18 (m, 2H), 7.19-7.14 | |
| | | | (m, 2H), 7.12 (m, 1H), 6.89 (m, 2H), 5.18 (s, 2H). | |
| 96 | B | 355.2 | 1H NMR (400 MHz, DMSO-d6) δ 13.19 (s, 1H), | 67 |
| | | | 8.94-8.66 (m, 2H), 8.15-8.04 (m, 2H), 7.78 (s, | |
| | | | 1H), 7.64-7.40 (m, 3H), 7.23-7.08 (m, 1H), | |
| | | | 6.93 (d, J = 8.1 Hz, 1H). | |
| 97 | C | 485.2 | 1H NMR (400 MHz, DMSO-d6) δ 13.04 (s, 1H), | 10 |
| | | | 12.15 (s, 1H), 8.08 (s, 1H), 7.90 (d, J = 5.2 Hz, | |
| | | | 1H), 7.81 (t, J = 7.8 Hz, 1H), 7.66 (t, J = 7.9 Hz, | |
| | | | 1H), 7.53 (s, 1H), 7.47 (d, J = 8.5 Hz, 1H), 7.33 | |
| | | | (d, J = 8.5 Hz, 1H), 6.62 (d, J = 8.1 Hz, 1H), 4.22- | |
| | | | 4.06 (m, 2H), 3.76 (m, 1H), 3.70-3.39 (m, | |
| | | | 6H), 2.20 (s, 3H). | |
| 98 | C | 375.2 | ¹H NMR (400 MHz, DMSO-d₆) δ 13.05 (s, 1H), | 5 |
| | | | 8.33 (s, 1H), 8.24 (s, 1H), 7.71 (d, J = 3.3 Hz, | |
| | | | 1H), 7.54 (d, J = 3.3 Hz, 1H), 7.50 (m, 2H), 7.33 | |
| | | | (d, J = 8.5 Hz, 1H), 2.17 (s, 3H). | |
| 99 | C | 387.2 | | 17 |
| 100 | B | 477.3 | 1H NMR (400 MHz, DMSO-d6) δ 13.21 (s, 1H), | 5 |
| | | | 12.55 (s, 1H), 9.31 (s, 1H), 8.74 (d, J = 4.7 Hz, | |
| | | | 1H), 8.50 (d, J = 8.1 Hz, 1H), 8.41 (s, 1H), 8.16 | |
| | | | (s, 1H), 7.81 (s, 1H), 7.76 (s, 1H), 7.64-7.55 (m, | |
| | | | 2H), 7.50-7.42 (m, 1H), 7.17 (d, J = 7.0 Hz, | |
| | | | 1H), 6.91 (d, J = 8.0 Hz, 2H), 5.57 (m, 1H), 4.89 | |
| | | | (m, 4H). | |
| 101 | C | 485.2 | 1H NMR (400 MHz, DMSO-d6) δ 13.04 (s, 1H), | 11 |
| | | | 12.12 (s, 1H), 8.03 (s, 1H), 7.90 (d, J = 5.5 Hz, | |
| | | | 1H), 7.83 (t, J = 7.9 Hz, 1H), 7.65 (t, J = 7.8 Hz, | |
| | | | 1H), 7.52 (s, 1H), 7.47 (d, J = 8.5 Hz, 1H), 7.32 | |
| | | | (d, J = 8.6 Hz, 1H), 6.61 (d, J = 8.1 Hz, 1H), 4.13 | |
| | | | (h, J = 7.0, 6.4 Hz, 2H), 3.75 (s, 1H), 3.70-3.39 | |
| | | | (m, 6H), 2.20 (s, 3H). | |
| 102 | C | 477.3 | 1H NMR (400 MHz, DMSO-d6) δ 13.06 (s, 1H), | 10 |
| | | | 12.10 (s, 1H), 8.09-7.96 (m, 3H), 7.88 (d, J = | |
| | | | 7.7 Hz, 1H), 7.65 (t, J = 7.8 Hz, 1H), 7.54 (d, J = | |
| | | | 8.5 Hz, 1H), 7.44-7.33 (m, 2H), 7.01 (s, 1H), | |
| | | | 6.70 (d, J = 8.1 Hz, 1H), 6.50 (t, J = 4.9 Hz, 1H), | |
| | | | 5.53-5.13 (m, 2H), 2.08 (s, 3H). | |
| 103 | B | 507.2 | 1H NMR (400 MHz, DMSO), d (ppm): 13.24 (s, | 8 |
| | | | 1H); 12.54 (br s, 1H); 9.04 (d, J = 2.2 Hz, 1H); | |
| | | | 8.53 (dd, J = 8.8, 2.2 Hz, 1H); 7.83 (s, 1H); 7.68 | |
| | | | (s, 1H); 7.59 (d, J = 8.4 Hz, 1H); 7.47 (t, J = 7.7 | |
| | | | Hz, 1H); 7.16 (d, 6.8 Hz, 1H); 7.04 (d, J = 8.8 Hz, | |
| | | | 1H); 7.01 (br s, 2H); 4.48 (t, J = 4.5 Hz, 2H); 3.70 | |
| | | | (t, J = 4.5 Hz, 2H); 3.31 (s, 3H). | |
| 104 | B | 547.1 | 1H NMR (400 MHz, DMSO), d (ppm): 13.25 (s, | 2 |
| | | | 1H); 9.04 (d, J = 2.2 Hz, 1H); 8.86 (br s, 1H); | |
| | | | 8.55 (dd, J = 8.7, 2.2 Hz, 1H); 8.41 (br s, 1H); | |
| | | | 8.14 (s, 1H); 7.83 (s, 1H); 7.70 (s, 1H); 7.60 (d, J = | |
| | | | 8.4 Hz, 1H); 7.47 (t, J = 7.7 Hz, 1H); 7.16 (d, J = | |
| | | | 6.9 Hz, 1H); 7.06 (d, J = 8.7 Hz, 1H); 6.96 (s, | |
| | | | 1H); 4.55 (t, J = 5.4 Hz, 2H); 3.39-3.27 (m, 4H); | |
| | | | 2.91-2.82 (m, 4H); 1.82-1.77 (m, 4H). | |
| 105 | D | 337.3 | ¹H NMR (400 MHz, DMSO-d₆) δ 9.26 (br s, 1H), | 641 |
| | | | 8.39 (s, 1H), 7.10 (d, J = 8.3 Hz, 1H), 7.00 (s, | |
| | | | 1H), 6.70 (dd, J = 8.2, 2.6 Hz, 1H), 6.54 (d, J = | |
| | | | 2.6 Hz, 1H), 5.94-5.80 (m, 1H), 5.58 (br s, 2H), | |
| | | | 4.41 (s, 2H), 4.19 (q, J = 2.7 Hz, 2H), 3.77 (t, J = | |
| | | | 5.4 Hz, 2H), 2.42-2.30 (m, 2H), 1.97 (s, 3H). | |
| 106 | C | 358.1 | ¹H NMR (400 MHz, DMSO-d6) δ 13.25 (s, 1H), | 24 |
| | | | 12.30 (s, 1H), 8.11 (d, J = 3.2 Hz, 1H), 7.81 (s, | |
| | | | 1H), 7.67 (s, 1H), 7.59 (d, J = 8.4 Hz, 2H), 7.46 | |
| | | | (t, J = 8.0 Hz, 1H), 7.13 (d, J = 7.2 Hz, 1H). | |
| 107 | C | 386.3 | ¹H NMR (400 MHz, DMSO-d6) δ 13.28 (s, 1H), | 3 |
| | | | 12.55 (s, 1H), 8.69 (s, 1H), 8.41 (s, 1H), 8.34 (s, | |
| | | | 1H), 7.84 (s, 1H), 7.63 (d, J = 8.0 Hz, 1H), 7.50 | |
| | | | (t, J = 7.2 Hz, 1H), 7.20 (d, J = 6.8 Hz, 1H). | |
| 108 | D | 333.3 | ¹H NMR (400 MHz, DMSO-d₆) δ 9.31 (s, 1H), | 73 |
| | | | 8.80 (d, J = 4.8 Hz, 2H), 8.49 (s, 1H), 8.13 (s, | |
| | | | 1H), 7.26 (t, J = 4.8 Hz, 1H), 7.14 (dd, J = 8.2, | |
| | | | 0.8 Hz, 1H), 6.74 (dd, J = 8.2, 2.6 Hz, 1H), 6.60 | |
| | | | (d, J = 2.6 Hz, 1H), 6.09 (br s, 2H), 4.72 (s, 2H), | |
| | | | 2.01 (s, 3H). | |
| 109 | C | 278.2 | 1H NMR (400 MHz, DMSO-d6) δ 13.16 (s, 1H), | 122 |
| | | | 11.94 (s, 1H), 7.92 (s, 1H), 7.75 (d, J = 1.0 Hz, | |
| | | | 1H), 7.53 (dt, J = 8.4, 1.0 Hz, 1H), 7.47-7.37 | |
| | | | (m, 2H), 7.09 (dd, J = 7.0, 0.8 Hz, 1H), 6.79 (d, J = | |
| | | | 8.1 Hz, 3H). | |
| 110 | C | 469.4 | | 7 |
| 111 | A | 438.1 | ¹H-NMR (DMSO-d₆, 400 MHz): δ 3.11 (2H, t, J = | 4 |
| | | | 7.4 Hz), 3.4 (2H, J = 8.4 hz), 7.18 (1H, d, J = 6.9 | |
| | | | Hz), 7.48 (1H, t, J = 7.7 Hz), 7.61-7.56 (3H, m), | |
| | | | 7.77 (1H, dJ, = 3.3 Hz), 7.81(1H, s), 8.08 (1H, | |
| | | | d, J = 7.4 Hz), 8.50 (1H, s), 8.55 (1H, dJ, = 4.9 | |
| | | | Hz), 8.69 (1H, s), 12.38 (1H, s), 13.24 (1H, br .s). | |
| 112 | A | 466.1 | ¹H-NMR (DMSO-d₆, 300 MHz): δ 7.23 (1H, d, J = | 34 |
| | | | 5.9 Hz), 7.52 (1H, dd, J = 8.5, 6.9 Hz), 7.64 (1H, | |
| | | | d, J = 8.4 Hz), 7.71-7.66 (2H, m), 7.83 (1H, dd, J = | |
| | | | 3.3, 0.6 Hz), 7.86 (1H, s), 8.57 (1H, s), 8.70 | |
| | | | (1H, dt, J = 8.1, 2.0 Hz), 8.83 (1H, dd, J = 4.8, | |
| | | | 1.5 Hz), 9.53 (1H, d, J = 2.3 Hz), 12.84 (1H, | |
| | | | s), 13.27 (1H, s). | |
| 113 | A | 452.1 | ¹H -NMR (DMSO-d₆, 400 MHz): δ 2.69 (3H, s), | 94 |
| | | | 7.21 (1H, d, J = 7.0 Hz), 7.51-7.42 (3H, m), 7.63 | |
| | | | (1H, d, J = 8.4 Hz), 7.75 (1H, d, J = 3.3 Hz), 7.85 | |
| | | | (1H, s), 8.04 (1H, d, J = 7.8 Hz), 8.54 (1H, s), | |
| | | | 8.65 (1H, d, J = 4.8 Hz), 12.67 (1H, br s), 13.26 | |
| | | | (1H, s). | |
| 114 | C | 395.2 | ¹H NMR (400 MHz, DMSO-d₆) δ 13.25 (bs, 1H), | 28 |
| | | | 8.36 (s, 1H), 8.35 (s, 1H), 8.29 (s, 1H), 7.82 (s, | |
| | | | 1H), 7.72 (s, 1H), 7.59 (d, J = 7.6 Hz, 1H), 7.48 | |
| | | | (t, J = 6.8 Hz, 1H), 7.18 (d, J = 6.4 Hz, 1H). | |
| 115 | C | 429.5 | ¹H NMR (400 MHz, DMSO-d6) δ 13.28 (bs, 1H), | 109 |
| | | | 12.59 (s, 1H), 8.50 (s, 1H), 8.40 (d, J = 3.2 Hz, | |
| | | | 1H), 8.30 (s, 1H), 7.84 (s, 1H), 7.63 (d, J = 8.4 | |
| | | | Hz, 1H), 7.49 (t, J = 7.6 Hz, 1H), 7.20 (d, J = 6.8 | |
| | | | Hz, 1H), | |
| 116 | C | 395.2 | ¹H NMR (400 MHz, DMSO-d₆) δ 13.27 (s, 1H), | 13 |
| | | | 12.48 (bs, 1H), 8.36 (s, 1H), 8.30 (s, 1H), 7.83 (s, | |
| | | | 1H), 7.62 (d, J = 8.0 Hz, 1H), 7.54-7.47 (m, | |
| | | | 2H), 7.19 (d, J = 6.8 Hz, 1H). | |
| 117 | C | 378.5 | ¹H NMR (400 MHz, MeOD) δ 8.06 (s, 2H), 7.88 | 15 |
| | | | (s, 2H), 7.63 (d, J = 8.4 Hz, 1H), 7.55 (t, J = 7.2 | |
| | | | Hz, 1H 1H), 7.25 (d, J = 6.8 Hz, 1H), 7.01 (s, | |
| | | | 1H), 6.44 (s, 1H). | |
| 118 | C | 385.2 | ¹H NMR (400 MHz, DMSO d₆) δ 13.26 (s, 1H), | 11 |
| | | | 12.46 (s, 1H), 8.29 (d, J = 3.2 Hz, 1H), 8.17 (s, | |
| | | | 1H), 7.83 (s, 2H), 7.79 (d, J = 4 Hz, 1H), 7.74 (d, | |
| | | | J = 4 Hz, 1H), 7.62 (d, J = 8.4 Hz, 1H), 7.49 (t, J = | |
| | | | 7.2 Hz, 1H), 7.19 (d, J = 7.2 Hz, 1H). | |
| 119 | C | 429.3 | ¹H NMR (400 MHz, DMSO-d6) δ 13.28 (bs, 1H), | 55 |
| | | | 12.54 (d, J = 2.8 Hz, 1H), 8.41 (s, 1H), 8.35 (d, J = | |
| | | | 3.2 Hz, 1H), 8.30 (s, 1H), 7.83 (s, 1H), 7.63 (d, | |
| | | | J = 8.4 Hz, 1H), 7.50 (t, J = 7.2 Hz, 1H), 7.19 (d, | |
| | | | J = 7.2 Hz, 1H). | |
| 120 | C | 374.2 | ¹H NMR (400 MHz, DMSO-d6) δ 13.31 (bs, 1H), | 189 |
| | | | 8.95 (s, 2H), 8.63 (s, 1H), 8.41 (s, 1H), 7.28 (s, | |
| | | | 1H), 7.63 (d, J = 8.4 Hz, 1H), 7.49 (t, J = 7.2 Hz, | |
| | | | 1H), 7.20 (t, J = 10 Hz, 1H). | |
| 121 | C | 374.3 | ¹H NMR (400 MHz, DMSO d₆) δ 13.24 (s, 1H), | 62 |
| | | | 12.20 (s, 1H), 8.11 (s, 1H), 8.84-7.82 (m, 2H), | |
| | | | 7.61 (d, J = 8.4 Hz, 1H), 7.49 (t, J = 7.2 Hz, 1H), | |
| | | | 7.30 (d, J = 3.6 Hz, 1H), 7.20 (d, J = 6.8 Hz, 1H), | |
| | | | 7.09 (bs, 2H), 6.79 (s, 1H), 2.32 (s, 3H). | |
| 122 | C | 356.5 | ¹H NMR (400 MHz, DMSO d₆) δ 14.27 (s, 1H), | 170 |
| | | | 13.26 (s, 1H), 9.51 (bs, 1H), 8.85 (s, 2H), 8.65 (d, | |
| | | | J = 5.2 Hz, 1H), 8.53 (s, 1H), 7.83 (s, 1H), 7.61 | |
| | | | (s, 1H), 7.48 (d, J = 6.4 Hz, 1H), 7.35 (d, J = 4.4 | |
| | | | Hz, 1H), 7.18 (s, 1H), 6.09 (bs, 1H). | |
| 123 | C | 394.2 | ¹H NMR (400 MHz, DMSO-d6) δ 13.24 (s, 1H), | 89 |
| | | | 12.30 (s, 1H), 8.19 (d, J = 3.6 Hz, 1H), 7.97 (s, | |
| | | | 1H), 7.82 (s, 1H), 7.60 (d, J = 8.4 Hz, 1H), 7.48(t, | |
| | | | J = 6.8 Hz, 1H), 7.40 (d, J = 4 Hz, 1H), 7.18 (d, J = | |
| | | | 6.8 Hz, 1H), 6.98 (d, J = 4.4 Hz, 1H). | |
| 124 | C | 390.3 | ¹H NMR (400 MHz, DMSO-d6) δ 13.23 (s, 1H), | 66 |
| | | | 12.21 (s, 1H), 8.07 (s, 1H), 7.92 (s, 1H), 7.61 (d, | |
| | | | J = 8.4 Hz, 1H), 7.56 (s, 1H), 7.48 (t, J = 8.0 Hz, | |
| | | | 1H), 7.33 (bs, 1H), 7.32 (bs, 1H), 7.23 (d, J = 6.8 | |
| | | | Hz, 1H), 7.113 (t, J = 9.2 Hz, 1H). | |
| 125 | C | 422.4 | ¹H NMR (400 MHz, DMSO-d6) δ 13.24 (s, 1H), | 839 |
| | | | 12.20 (s, 1H), 8.02 (s, 1H), 7.91 (s, 1H), 7.79 (s, | |
| | | | 2H), 7.71 (d, J = 6.8 Hz, 2H), 7.55-7.59 (m, 3H), | |
| | | | 7.48 (s, 2H), 7.24 (s, 1H). | |
| 126 | C | 428.4 | ¹H NMR (400 MHz, MeOD) δ 8.10 (s, 1H), 8.03 | 880 |
| | | | (s, 1H), 7.09 (s, 1H), 7.64 (d, J = 8.4 Hz, 1H), | |
| | | | 7.55 (t, J = 7.2 Hz, 1H), 7.43 (s, 1H), 7.38 (s, | |
| | | | 1H), 7.63 (d, J = 8.4 Hz, 1H), 7.26 (d, J = 6.8 Hz, 1H). | |
| 127 | C | 316.3 | ¹H NMR (400 MHz, DMSO d₆) δ 13.21 (s, 1H), | 34 |
| | | | 12.04 (s, 1H), 8.05 (s, 1H), 7.76 (s, 1H), 7.57 (d, | |
| | | | J = 8.4 Hz, 1H), 7.47-7.43 (t, 1H), 7.13 (d, J = 6.4 | |
| | | | Hz, 1H), 6.96 (s, 1H), 6.67 (bs, 2H), 2.68-2.66 | |
| | | | (m, 1H), 0.88 (m, 2H), 0.60-0.59 (m, 2H). | |
| 128 | C | 386.4 | ¹H NMR (400 MHz, DMSO-d₆) δ 13.29 (bs, 1H), | 5 |
| | | | 12.56 (s, 1H), 8.70 (s, 1H), 8.41 (s, 1H), 8.35 (d, | |
| | | | J = 3.6 Hz, 1H), 7.84 (s, 1H), 7.63 (d, J = 8.4 Hz, | |
| | | | 1H), 7.50 (t, J = 7.2 Hz, 1H), 7.20 (d, J = 6.8 Hz, 1H). | |
| 129 | C | 358.3 | ¹H NMR (400 MHz, DMSO d₆) δ 13.25 (s, 1H), | 23 |
| | | | 12.30-12.14 (bs, 1H), 8.15-8.07 (m, 2H), 7.81 (s, | |
| | | | 1H), 7.65-7.60 (m, 2H), 7.49 (t, J = 7.2 Hz, 1H), | |
| | | | 7.17 (d, J = 6.8 Hz, 1H), 7.04 (s, 1H), 3.83 (s, 3H). | |
| 130 | C | 356.5 | ¹H NMR (400 MHz, DMSO d₆) δ 13.26 (s, 1H), | 45 |
| | | | 12.32 (s, 1H), 9.42 (s, 1H), 8.62 (s, 1H), 8.46 (s, | |
| | | | 1H), 8.14 (s, 1H), 8.02 (s, 1H), 7.78 (d, 1H), 7.62 | |
| | | | (d, J = 8.4 Hz, 1H), 7.49 (t, J = 7.2 Hz, 1H), 7.20 | |
| | | | (d, J = 6.8 Hz, 1H), 6.97 (s, 1H). | |
| 131 | C | 356.5 | ¹H NMR (400 MHz, DMSO d₆) δ 13.26 (s, 1H), | 70 |
| | | | 12.34 (s, 1H), 9.56 (s, 1H), 9.17 (s, 1H), 8.12 (s, | |
| | | | 1H), 7.93 (s, 2H), 7.72 (s, 1H), 7.62 (d, J = 8.0 | |
| | | | Hz, 1H), 7.49 (t, J = 7.2 Hz, 1H), 7.24 (d, J = 6.8 | |
| | | | Hz, 1H). | |
| 132 | C | 361.2 | ¹H NMR (400 MHz, DMSO d₆) δ 13.27 (s, 1H), | 4 |
| | | | 12.46-12.45 (s, 1H), 8.338 (s, 1H), 8.33 (d, J = | |
| | | | 3.6 Hz, 1H), 8.20 (s, 1H), 7.86 (d, J = 1.2 Hz, | |
| | | | 1H), 7.85 (s, 1H), 7.65-7.64 (d, J = 8.4 Hz, 1H), | |
| | | | 7.51 (t, J = 7.2 Hz, 1H), 7.21 (d, J = 6.8 Hz, 1H). | |
| 133 | C | 370.3 | ¹H NMR (400 MHz, DMSO-d6) δ 13.10 (s, 1H), | 26 |
| | | | 12.29 (s, 1H), 8.11 (s, 1H), 7.50- 7.54 (m, 3H), | |
| | | | 7.34 (d, J = 8.4 Hz, 1H), 6.70 (bs, 1H), 2.18 (s, 3H). | |
| 134 | B | 399.3 | 1H NMR (400 MHz, DMSO-d6) δ 12.39 (s, 1H), | 5 |
| | | | 9.15 (s, 1H), 8.76 (d, J = 5.0 Hz, 2H), 8.28-8.05 | |
| | | | (m, 2H), 6.93 (d, J = 8.2 Hz, 1H), 6.73 (d, J = 8.2 | |
| | | | Hz, 1H), 6.62 (s, 1H), 6.18 (s, 2H), 2.82 (m, 1H), | |
| | | | 1.81 (s, 3H), 1.75 (s, 3H), 0.91 (m, 2H), 0.60 (m, 2H). | |
| 135 | B | 465.3 | 1H NMR (400 MHz, DMSO-d6) δ 12.48 (s, 1H), | 3 |
| | | | 9.17 (s, 1H), 8.87-8.71 (m, 2H), 8.26 (s, 1H), | |
| | | | 8.10-8.02 (m, 3H), 7.45 (s, 1H), 6.96 (d, J = 8.2 | |
| | | | Hz, 1H), 6.76 (d, J = 8.1 Hz, 1H), 6.46 (bs, 1H), | |
| | | | 3.70 (m, 1H), 1.88 (s, 3H), 1.81 (s, 3H), 1.08- | |
| | | | 0.96 (m, 2H), 0.96-0.82 (m, 2H). | |
| 136 | D | 332.3 | ¹H NMR (400 MHz, DMSO-d₆) δ 9.33 (br s, 1H), | 548 |
| | | | 8.60 (ddd, J = 4.8, 1.9, 1.0 Hz, 1H), 8.44 (s, 1H), | |
| | | | 7.86-7.74 (m, 2H), 7.67 (s, 1H), 7.21 (ddd, J = | |
| | | | 7.1, 4.8, 1.3 Hz, 1H), 7.13 (dd, J = 8.2, 0.7 Hz, | |
| | | | 1H), 6.73 (dd, J = 8.2, 2.6 Hz, 1H), 6.62 (d, J = | |
| | | | 2.6 Hz, 1H), 5.85 (br s, 2H), 4.69 (s, 2H), 2.02 (s, 3H). | |
| 137 | C | 345.2 | ¹H NMR (400 MHz, DMSO d₆) δ 12.16 (bs, 1H), | 51 |
| | | | 9.36 (bs, 1H), 8.55 (s, 1H), 8.14 (d, J = 7.6 Hz, | |
| | | | 1H), 8.03 (s, 1H), 7.75 (s, 2H), 7.21 (bs, 1H), | |
| | | | 7.14 (d, J = 8.4 Hz, 1H), 6.73 (d, J = 6 Hz, 1H), | |
| | | | 6.59 (s, 1H), 2.00 (s, 3H). | |
| 138 | C | 346.2 | ¹H NMR (400 MHz, DMSO-d6) δ 11.98 (s, 1H), | 41 |
| | | | 9.34 (s, 1H), 8.07 (s, 1H), 7.44 (s, 1H), 7.13 (d, J = | |
| | | | 8.4 Hz, 1H), 6.73 (dd, J = 8.4, 2.0 Hz, 1H), 6.54 | |
| | | | (d, J = 2.0 Hz, 1H), 1.94 (s, 1H). | |
| 139 | D | 352.2 | ¹H NMR (400 MHz, DMSO-d₆) δ 9.17 (s, 1H), | 7 |
| | | | 8.49 (s, 1H), 7.82 (d, J = 3.3 Hz, 1H), 7.60 (d, J = | |
| | | | 3.3 Hz, 1H), 7.41 (s, 1H), 6.94 (d, J = 8.2 Hz, | |
| | | | 1H), 6.75 (d, J = 8.2 Hz, 1H), 5.81 (s, 2H), 4.59 | |
| | | | (s, 2H), 1.86 (s, 3H), 1.79 (s, 3H). | |
| 140 | D | 364.2 | ¹H NMR (400 MHz, DMSO-d₆) δ 9.13 (s, 1H), | 111 |
| | | | 8.56 (d, J = 2.4 Hz, 1H), 8.39 (d, J = 3.9 Hz, 2H), | |
| | | | 7.59 (dd, J = 6.9, 5.0 Hz, 1H), 7.03 (d, J = 2.1 | |
| | | | Hz, 1H), 6.92 (d, J = 8.2 Hz, 1H), 6.72 (d, J = 8.2 | |
| | | | Hz, 1H), 5.67 (s, 2H), 4.36 (s, 2H), 1.87 (s, 3H), | |
| | | | 1.80 (s, 3H). | |
| 141 | D | 347.2 | ¹H NMR (400 MHz, DMSO-d₆) δ 9.14 (s, 1H), | 92 |
| | | | 8.46 (s, 1H), 7.01 (s, 1H), 6.91 (d, J = 8.2 Hz, | |
| | | | 1H), 6.72 (d, J = 8.2 Hz, 1H), 5.37 (s, 2H), 4.18 | |
| | | | (d, J = 1.1 Hz, 2H), 1.82 (s, 3H), 1.75 (s, 3H). | |
| 142 | D | 421.4 | ¹H NMR (400 MHz, DMSO d₆) δ 11.64 (bs, 1H), | 2240 |
| | | | 9.34 (s, 1H), 8.52 (s, 1H), 7.51 (s, 1H), 7.43 (s, | |
| | | | 1H), 7.13 (d, J = 8.4 Hz, 1H), 6.73 (d, J = 8.0 Hz, | |
| | | | 1H), 6.59 (s, 1H), 5.93 (bs, 1H), 4.55 (s, 2H), | |
| | | | 2.02 (s, 3H), 1.78 (bs, 1H), 0.86 (d, J = 8.4 Hz, 4H). | |
| 143 | D | 437.0 | 1H NMR (400 MHz, DMSO d6) δ 9.38 (s, 1H), | 1600 |
| | | | 8.55 (s, 1H), 7.72 (s, 1H), 7.50 (s, 1H), 7.15 (d, J = | |
| | | | 8.0 Hz, 1H), 6.76 (dd, J = 8.0, 2.0 Hz, 1H), 6.60 | |
| | | | (d, J = 2.0 Hz, 1H), 6.03 (bs, 2H), 4.59 (s, 2H), | |
| | | | 3.73 (s, 2H), 3.57 (s, 4H), 2.42 (s, 4H), 2.02 (s, 2H). | |
| 144 | D | 465.3 | ¹H NMR (400 MHz, DMSO d₆) δ 10.85 (s, 1H), | 930 |
| | | | 9.36 (s, 1H), 8.67 (s, 1H), 7.65 (s, 1H), 7.48 (s, | |
| | | | 1H), 7.13 (d, J = 8.0 Hz, 1H), 6.73 (d, J = 6.4 Hz, | |
| | | | 1H), 6.58 (d, 1H), 6.08 (s, 1H), 4.61 (s, 2H), 3.90 | |
| | | | (d, J = 10.0 Hz, 2H), 3.34 (t, J = 12 Hz, 2H), 2.00 | |
| | | | (s, 3H), 1.65 (m, 4H). | |
| 145 | D | 425.2 | ¹H NMR (400 MHz, DMSO d₆) δ 9.40 (s, 1H), | 645 |
| | | | 8.67 (s, 1H), 8.32 (t, J = 5.6 Hz, 1H), 8.19 (s, | |
| | | | 1H), 7.71 (s, 1H), 7.16 (d, J = 8.0 Hz, 1H), 6.77 | |
| | | | (d, J = 6.8 Hz, 1H), 6.62 (s, 1H), 6.11 (bs, 1H), | |
| | | | 4.80 (s, 1H), 4.73 (s, 1H), 3.52 (s, 2H), 3.39- | |
| | | | 3.34 (m, 2H), 2.02 (s, 3H). | |
| 146 | D | 437.3 | ¹H NMR (400 MHz, DMSO d₆) δ 9.36 (s, 1H), | 135 |
| | | | 8.57 (s, 1H), 7.54 (s, 1H), 7.41 (s, 1H), 7.15 (d, J = | |
| | | | 8.0 Hz, 1H), 6.76 (dd, J = 8.0, 2.0 Hz, 1H), 6.60 | |
| | | | (d, J = 2.0 Hz, 1H), 6.09 (bs, 2H), 4.60 (s, 2H), | |
| | | | 3.64 (s, 2H), 3.59 (s, 4H), 2.50 (s, 4H), 2.02 (s, 3H). | |
| 147 | D | 425.0 | ¹H NMR (400 MHz, DMSO d₆) δ 9.36 (s, 1H), | 209 |
| | | | 8.685 (t, J = 5.2 Hz, 2H), 8.40 (s, 1H), 7.61 (s, | |
| | | | 1H), 7.16 (d, J = 8.4 Hz, 1H), 6.77 (dd, J = 8.4, | |
| | | | 2.4 Hz, 1H), 6.61 (d, J = 4 Hz, 1H), 6.16 (bs, 2H), | |
| | | | 4.81 (t, J = 5.6 Hz, 1H), 4.61 (s, 2H), 3.51 (t, J = | |
| | | | 5.6 Hz, 2H), 3.29 (bs, 2H), 2.02 (s, 3H). | |
| 148 | D | 382.3 | ¹H NMR (400 MHz, DMSO d₆) δ 9.36 (s, 1H), | 1860 |
| | | | 8.62 (s, 1H), 8.23 (s, 1H), 7.62 (s, 1H), 7.16 (d, J = | |
| | | | 8.0 Hz, 1H), 6.76 (dd, J = 8.8, 2.0 Hz, 1H), 6.61 | |
| | | | (d, J = 2.4 Hz, 1H), 6.18 (bs, 1H), 4.63 (s, 2H), | |
| | | | 2.02 (s, 3H). | |
| 149 | D | 406.0 | ¹H NMR (400 MHz, DMSO-d6) δ 9.393 (s, 1H), | 682 |
| | | | 8.61 (s, 1H), 8.43 (s, 1H), 7.61 (s, 1H), 7.16 (d, J = | |
| | | | 8.4 Hz, 1H), 6.77 (dd, J = 8.0, 2.4 Hz, 1H), 6.62 | |
| | | | (d, J = 2.0 Hz, 1H) 6.2 (bs, 1H), 4.62 (s, 2H), | |
| | | | 2.02 (s, 3H). | |
| 150 | D | 395.2 | ¹H NMR (400 MHz, DMSO d₆) δ 9.51 (s, 1H), | 163 |
| | | | 8.63-8.53 (m, 2H), 8.34 (s, 1H), 7.60 (s, 1H), | |
| | | | 7.15 (d, J = 8.0 Hz, 1H), 6.77 (d, J = 8.0 Hz, 1H), | |
| | | | 6.61 (s, 1H), 6.17 (bs, 1H), 4.62 (s, 2H), 2.78 (s, | |
| | | | 3H), 2.02 (s, 1H). | |
| 151 | D | 409.3 | ¹H NMR (400 MHz, DMSO d₆) δ 9.36 (s, 1H), | 61 |
| | | | 8.58 (s, 1H), 8.011 (s, 1H), 7.53 (s, 1H), 7.14 (d, | |
| | | | J = 8.4 Hz, 1H), 6.75 (dd, J = 8.4, 2.4 Hz, 1H), | |
| | | | 6.6 (d, J = 2.4 Hz, 1H), 6.102 (bs, 2H), 4.63 (s, | |
| | | | 2H), 3.21 (s, 3H), 3.011 (s, 3H), 2.02 (s, 3H). | |
| 152 | D | 451.2 | ¹H NMR (400 MHz, DMSO d₆) δ 9.37 (s, 1H), | 683 |
| | | | 8.61 (s, 1H), 8.16 (s, 1H), 7.59 (s, 1H), 7.16 (d, J = | |
| | | | 8.4 Hz, 1H), 6.77 (d, J = 8.4 Hz, 1H), 6.61 (d, | |
| | | | 1H), 6.17 (bs, 1H), 4.63 (s, 2H), 3.66 (s, 8H), | |
| | | | 2.02 (s, 3H). | |
| 153 | D | 423.2 | ¹H NMR (400 MHz, DMSO d₆) δ 9.35 (s, 1H), | 477 |
| | | | 8.61 (s, 1H), 8.39 (m, 2H), 7.59 (s, 1H), 7.14 (d, J = | |
| | | | 8.4 Hz, 1H), 6.74 (dd, J = 8.0 Hz, 1H), 6.59 (d, | |
| | | | 1H), 4.60 (s, 2H), 4.04 (m, J = 4 Hz, 1H), 2.00 (s, | |
| | | | 3H), 1.16 (d, J = 6.8 Hz, 3H). | |
| 154 | D | 409.2 | ¹H NMR (400 MHz, DMSO d₆) δ 9.36 (s, 1H), | 298 |
| | | | 8.62 (s, 1H), 8.02 (s, 1H), 7.60 (s, 1H), 7.16 (d, J = | |
| | | | 8.0 Hz, 1H), 6.77 (dd, J = 8.0, 1.6 Hz, 1H), 6.60 | |
| | | | (d, J = 4 Hz, 1H), 6.17 (d, J = 12 Hz, 2H), 4.63 (s, | |
| | | | 2H), 3.25 (bs, 3H), 3.03 (bs, 3H), 2.027 (s, 3H). | |
| 155 | D | 363.0 | ¹H NMR (400 MHz, DMSO d₆) δ 9.41 (s, 1H), | 179 |
| | | | 8.68 (d, J = 6.4 Hz, 2H), 7.63 (s, 1H), 7.16 (d, J = | |
| | | | 8.4 Hz, 1H), 6.78 (d, J = 8.4 Hz, 1H), 6.61 (bs, | |
| | | | 2H), 4.63 (s, 2H), 2.02 (s, 3H). | |
| 156 | D | 372.2 | ¹H NMR (400 MHz, DMSO d₆) δ 9.366 (s, 1H), | 576 |
| | | | 8.606 (s, 1H), 7.89 (s, 1H), 7.47 (s, 1H), 7.15 (d, | |
| | | | J = 8.0 Hz, 1H), 6.77 (d, J = 8.0 Hz, 1H), 6.61 (d, | |
| | | | 1H), 6.13 (bs, 2H), 4.63 (s, 2H), 2.02 (s, 3H). | |
| 157 | D | 451.3 | ¹H NMR (400 MHz, DMSO d₆) δ 9.37 (s, 1H), | 244 |
| | | | 8.56 (s, 1H), 8.07 (s, 1H), 7.05 (s, 1H), 7.16 (d, J = | |
| | | | 8.4 Hz, 1H), 6.76 (d, J = 8.0 Hz, 1H), 6.62 (s, | |
| | | | 1H), 6.12 (bs, 1H), 4.62 (s, 2H), 3.84 (bs, 2H), | |
| | | | 3.67 (bs, 6H), 2.02 (s, 3H). | |
| 158 | D | 423.2 | ¹H NMR (400 MHz, DMSO d₆) δ 9.37 (s, 1H), | 55 |
| | | | 8.65 (s, 1H), 8.17 (s, 1H), 8.2 (d, J = 8.0 Hz 1H), | |
| | | | 7.71 (s, 1H), 7.16 (d, J = 8.4 Hz, 1H), 6.77 (d, J = | |
| | | | 8.4 Hz, 1H), 6.62 (s, 1H), 6.09 (bs, 1H), 4.74 (s, | |
| | | | 2H), 4.16-4.10 (m, 1H), 2.02 (s, 3H), 1.21 (d, J = | |
| | | | 6.4 Hz, 6H). | |
| 159 | D | 395.2 | ¹H NMR (400 MHz, DMSO d₆) δ 9.37 (s, 1H), | 75 |
| | | | 8.68 (s, 1H), 8.36 (d, J = 4.8 Hz, 1H), 8.16 (s, | |
| | | | 1H), 7.71 (s, 1H), 7.16 (d, J = 8.4 Hz, 1H), 6.77 | |
| | | | (dd, J = 8.4, 2.4 Hz, 1H), 6.62 (d, J = 2.4 Hz, 1H), | |
| | | | 6.09 (bs, 2H), 4.74 (s, 2H), 2.82 (d, J = 4.8 Hz, | |
| | | | 3H), 2.02 (s, 3H). | |
| 160 | D | 363.2 | ¹H NMR (400 MHz, DMSO d₆) δ 9.38 (s, 1H), | 237 |
| | | | 8.79 (s, 1H), 8.64 (s, 1H), 7.59 (s, 1H), 7.16 (d, J = | |
| | | | 8.4 Hz, 1H), 6.77 (dd, J = 8.0, 2.4 Hz, 1H), 6.61 | |
| | | | (d, J = 2.0 Hz, 1H), 6.24 (bs, 1H), 4.63 (s, 2H), | |
| | | | 2.02 (s, 3H). | |
| 161 | D | 372.2 | ¹H NMR (400 MHz, DMSO d₆) δ 9.39 (s, 1H), | 253 |
| | | | 8.61 (s, 1H), 7.67 (s, 1H), 7.54 (s, 1H), 7.16 (d, J = | |
| | | | 8.4 Hz, 1H), 6.76 (dd, J = 8.4, 2.4 Hz, 1H), 6.60 | |
| | | | (d, J = 2.4 Hz, 1H), 6.16 (bs, 1H), 4.60 (s, 2H), | |
| | | | 2.02 (s, 3H). | |
| 162 | D | 382.2 | ¹H NMR (400 MHz, DMSO d₆) δ 9.41 (bs, 1H), | 182 |
| | | | 8.59 (s, 1H), 8.23 (s, 1H), 7.57 (s, 1H), 7.14 (d, J = | |
| | | | 8.0 Hz, 1H), 6.75 (dd, J = 8.4 Hz, 1H), 6.60 (d, | |
| | | | 1H), 6.07 (bs, 2H), 4.62 (s, 2H), 2.01 (s, 3H). | |
| 163 | D | 331.3 | ¹H NMR (400 MHz, DMSO-d₆) δ 9.30 (br s, 1H), | 1070 |
| | | | 8.40 (s, 1H), 7.57-7.50 (m, 2H), 7.44-7.36 (m, | |
| | | | 2H), 7.33-7.24 (m, 1H), 7.16-7.09 (m, 2H), | |
| | | | 6.72 (dd, J = 8.2, 2.6 Hz, 1H), 6.61 (d, J = 2.6 | |
| | | | Hz, 1H), 5.64 (br s, 2H), 4.57-4.35 (m, 2H), | |
| | | | 2.02 (s, 3H). | |
| 164 | E | 350.3 | ¹H NMR (400 MHz, DMSO-d₆) δ 11.28 (br d, J = | 12 |
| | | | 5.7 Hz, 1H), 9.37 (br s, 1H), 7.88 (d, J = 3.4 Hz, | |
| | | | 1H), 7.68 (d, J = 3.4 Hz, 1H), 7.65 (dd, J = 7.5, | |
| | | | 1.2 Hz, 1H), 7.43 (s, 1H), 7.18 (dd, J = 7.5, 5.8 | |
| | | | Hz, 1H), 7.15 (d, J = 8.6 Hz, 1H), 6.75 (dd, J = | |
| | | | 8.3, 2.6 Hz, 1H), 6.61 (d, J = 2.6 Hz, 1H), 2.01 (s, 3H). | |
| 165 | G | 453.3 | ¹H NMR (400 MHz, DMSO-d6) δ 13.24 (s, 1H), | 596 |
| | | | 12.93 (bs, 1H), 8.37 (s, 1H), 7.79 (s, 1H), 7.60- | |
| | | | 7.58 (m, 2H), 7.56 (t, J = 7.6 Hz, 2H), 7.47 (t, J = | |
| | | | 8 Hz, 2H), 7.40-7.37 (m 3H), 7.19-7.15(m, 2H) | |

### Example 3. Genetic Validation

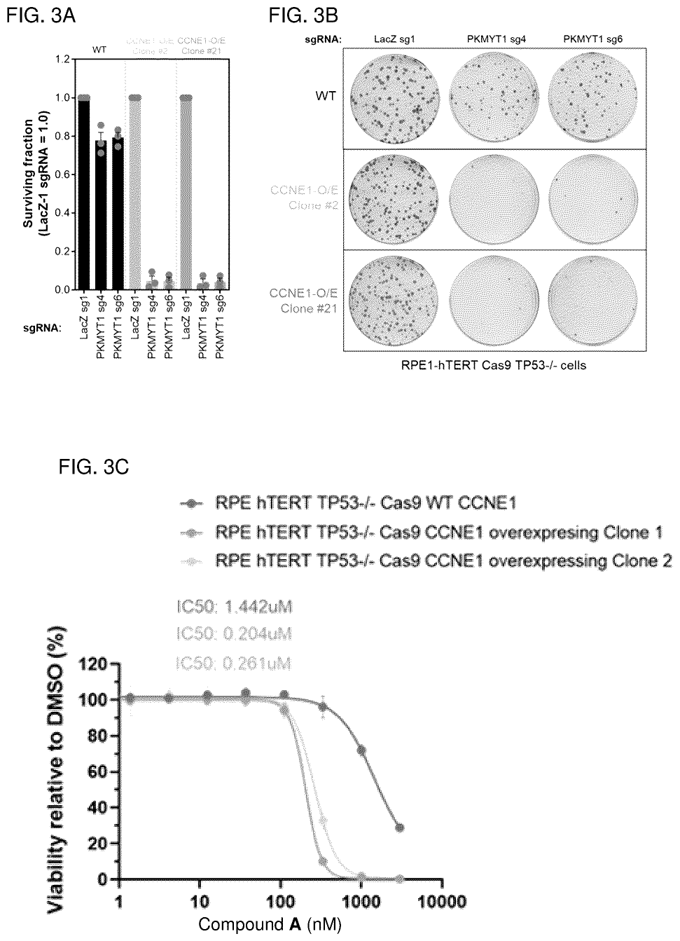

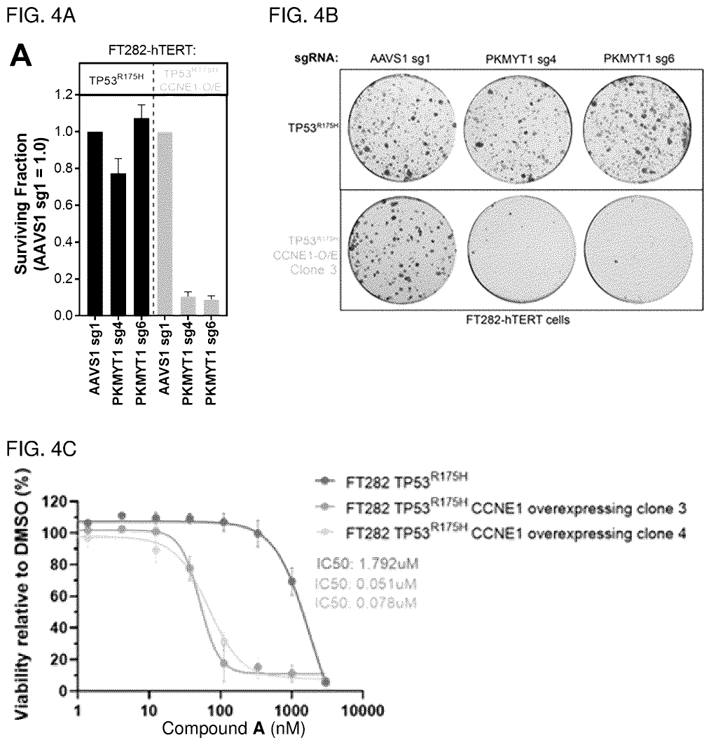

Two sgRNAs for PKMYT1 and one sgRNA for LacZ (control) were transduced into the RPE1-hTERT Cas9 TP53−/− parental (WT) and CCNE1-overexpressing clones. Infected cells were plated at low density to measure their ability to form colonies of <50 cells. After 10 days of growth, the colonies were stained, imaged, and quantified. Using clonogenic survival assays, we observed a profound cellular fitness defect in CCNE1-overexpressing cells compared to parental cells transduced with PKMYT1 sgRNAs (FIGS. 3A and 3B). This experiment was repeated using FT282-hTERT TP53−/− parental (WT) and CCNE1-overexpressing clones and similar results were observed (FIGS. 4A and 4B).

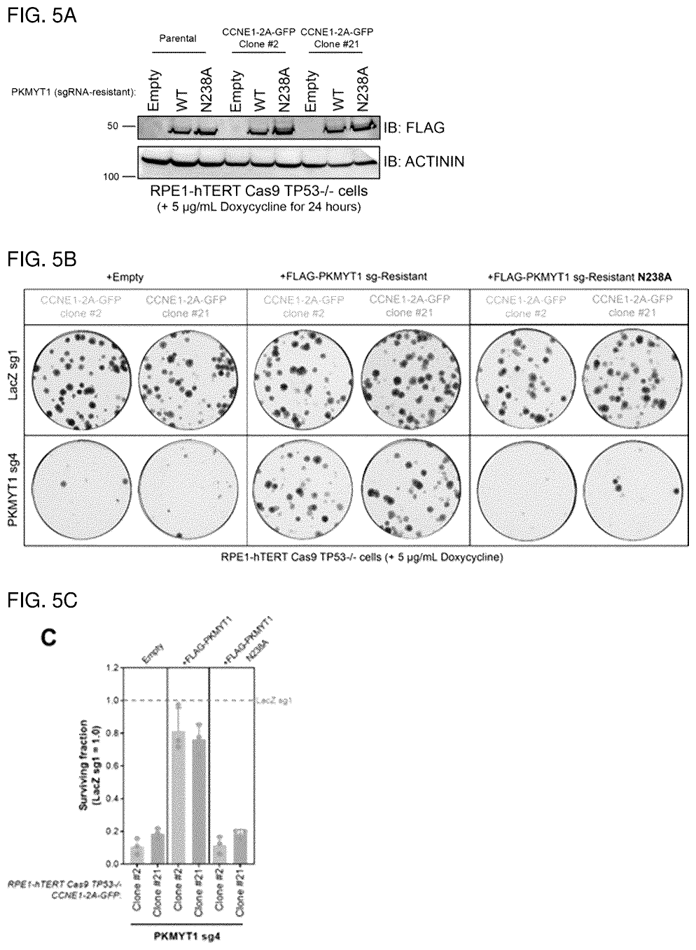

To determine if the kinase activity of PKMYT1 was responsible for maintaining the viability of CCNE1-overexpressing RPE1-hTERT Cas9 TP53−/− cells, the PKMYT1 open reading frame (ORF) was cloned into an inducible mammalian expression vector. sgRNA-resistant silent mutations in the PKMYT1 ORF sequence were then created by PCR mutagenesis. A single point mutation was generated that resulted in an asparagine (N) to alanine (A) amino acid change at residue 238. The N238A amino acid change in the kinase domain resulted in a catalytically inactive PKMYT1 mutant. Stable cell lines in the RPE1-hTERT Cas9 TP53−/− parental and CCNE1-overexpressing clones were generated that either expressed the wild type PKMYT1 ORF or the kinase-dead N238A mutant (FIG. 5A). These stable cell lines were transduced with either a LacZ non-targeting sgRNA or PKMYT1 sgRNA #4. The cells were then plated at low density to measure their ability to form colonies of >50 cells. After 10 days of growth, the colonies were stained, imaged, and quantified. Expression of an sgRNA-resistant PKMYT1 ORF but not the catalytic-dead version rescued the fitness defect induced by transduction of sgRNA #4 into both CCNE1-overexpressing clones (FIGS. 5B and 5C). This result demonstrated that targeting the kinase activity of PKMYT1 selectively kills CCNE1-overexpressing cells.

### Example 4. Pharmacological Validation

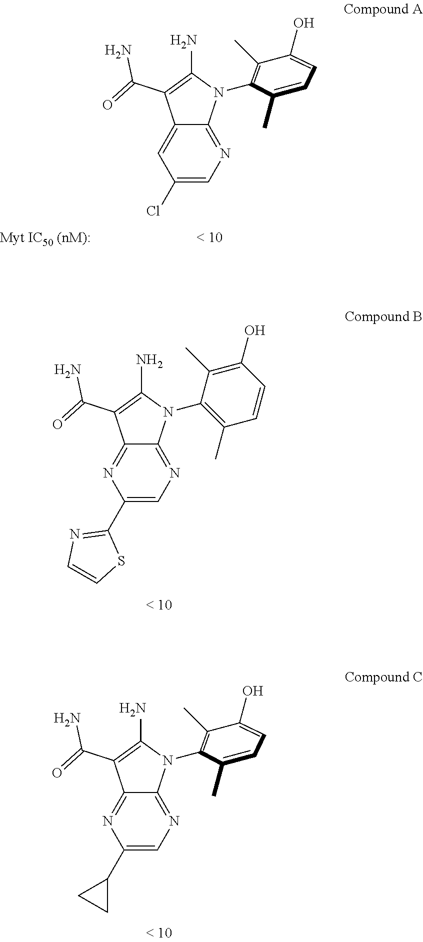

RPE1-hTERT Cas9 TP53−/− parental (WT) and CCNE1-overexpressing clones were treated with compound A in a dose titration and cell viability was determined. The CCNE1-overexpressing cells were found to be more sensitive to compound A than the corresponding WT cells (FIG. 3C). A similar effect was seen in FT282-hTERT TP53^{R175H} WT and CCNE1-overexpressing clones (FIG. 4C). For dose-response proliferation assays using RPE1-hTERT and FT282-hTERT cell lines, cells were seeded in 96-well plates and dosed with serially diluted Myt1 inhibitor. Cells were imaged once per day using the IncuCyte S3 microscope and percent well confluency was calculated over time. Once cells reached four population doublings the experiment was ended and IC₅₀ curves were plotted for the final time point. Percentage confluency was calculated relative to the cell confluency in the untreated wells.

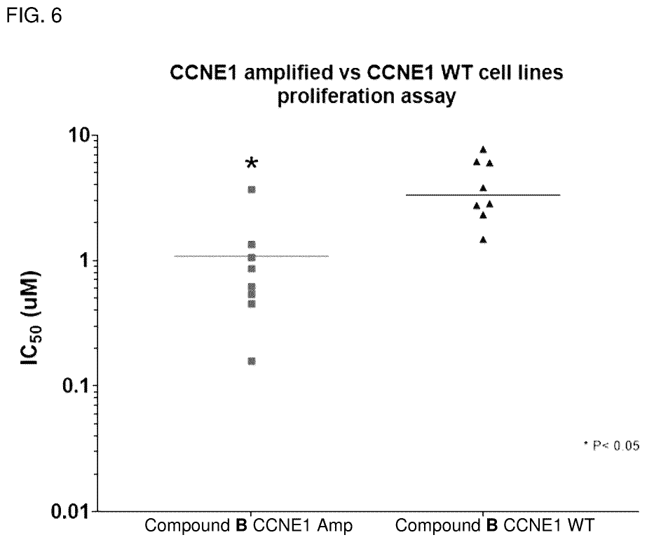

A panel of 16 cancer cell lines with either normal (n=8) or elevated levels of CCNE1 (n=8) was evaluated for their sensitivity to compound B in a cell proliferation assay (FIG. 6). Dose-response curves in these cancer cell line proliferation assays were generated as follows. Cells were seeded in 96-well plates and dosed with serially diluted compound B. After 7 days, Cell Titer Glo (CTG) was used to assess the proliferation status of these cells and the IC₅₀ values were plotted.

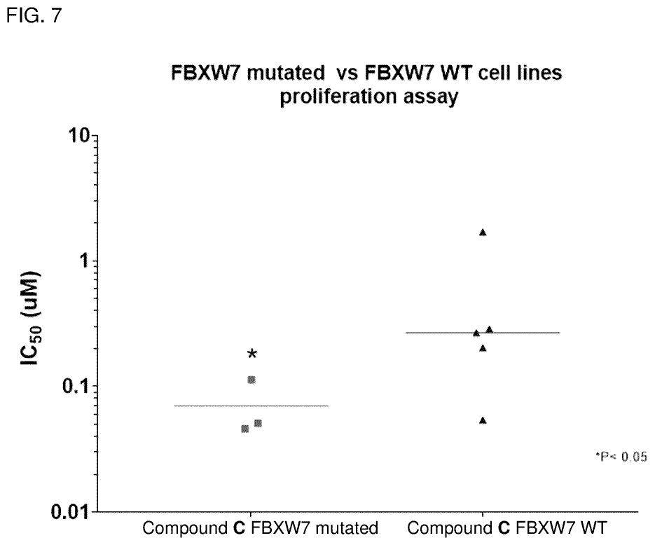

A similar experiment was conducted in a panel of 8 cancer cell lines with either wild-type FBXW7 (n=5) or FBXW7-mutations (n=3) in which these cells were evaluated for their sensitivity to compound C in a cell proliferation assay (FIG. 7). Dose-response curves in these cancer cell line proliferation assays were generated as follows. Cells were seeded in 96-well plates and dosed with serially diluted Myt1 inhibitor. Cells were imaged once per day using the IncuCyte S3 microscope and percent well confluency was calculated over time. Once cells reached four population doublings the experiment was ended and IC₅₀ curves were plotted for the final time point. Percentage confluency was calculated relative to the cell confluency in the untreated wells.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of formula (I): 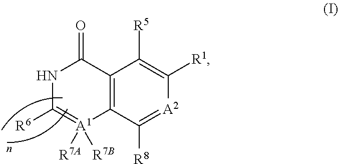
or a pharmaceutically acceptable salt thereof,
wherein
R¹ is: 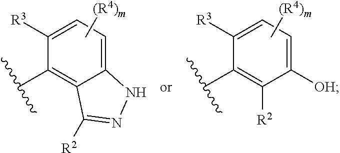 [symbol]
is a single bond, and n is 0 or 1; or [symbol] is a double bond, and n is 1;
m is 0, 1, or 2;
A¹ is N, R^{7A} is absent, hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂, and R^{7B} is absent; or A¹ is C, and R^{7A} is hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂, and R^{7B} is absent, hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{7C}, or —N(R^{7D})₂;
A² is N or CH;
each of R² and R³ are independently hydrogen, halogen, optionally substituted C₁₋₆ alkyl, or optionally substituted C₃₋₈ cycloalkyl;
each R⁴ is independently halogen;
R⁵ is hydrogen, halogen, hydroxyl, optionally substituted C₁₋₆ alkyl, —N(R^{5A})₂, or —OR^{5B};
each R^{5A} is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or optionally substituted C₁₋₆ alkylsulfonyl; or two R^{5A} groups, together with the atom to which both are attached, combine to form an optionally substituted C₂₋₉ heterocyclyl;
R^{5B} is optionally substituted C₁₋₆ alkyl;
R⁶ is hydrogen, halogen, optionally substituted C₁₋₆ alkyl, optionally substituted C₁₋₆ heteroalkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{6A}, —N(R^{6B})₂, or L¹-R^{6C};
R^{6A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{6B} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{6C} is hydrogen, halogen, optionally substituted C₁₋₆ alkyl, optionally substituted C₂₋₆ alkenyl, optionally substituted C₂₋₆ alkynyl, optionally substituted acyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{6D}, or —N(R^{6E})₂;
R^{6D} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{6E} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{7C} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{7D} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R⁸ is hydrogen, halogen, optionally substituted C₁₋₆ alkyl, optionally substituted C₂₋₆ alkenyl, optionally substituted C₂₋₆ alkynyl, optionally substituted optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{8A}, or -L²-R^{8B};
R^{8A} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
R^{8B} is hydrogen, halogen, cyano, optionally substituted C₁₋₆ alkyl, optionally substituted C₂₋₆ alkenyl, optionally substituted C₂₋₆ alkynyl, optionally substituted acyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, optionally substituted C₁₋₉ heteroaryl, —OR^{8C}, —N(R^{8D})₂, —CO₂H, —NHC(O)(R^{8E}), —C(O)N(R^{8E})₂;
R^{8C} is hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{8D} is independently hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₆₋₁₀ aryl, or optionally substituted C₁₋₉ heteroaryl;
each R^{8E} is independently hydrogen, optionally substituted C₁₋₆ alkyl, optionally substituted C₁₋₆ heteroalkyl, optionally substituted C₃₋₈ cycloalkyl, or optionally substituted C₂₋₉ heterocyclyl; or two R^{8E} groups, together with the atom to which both are attached, combine to form an optionally substituted C₂₋₉ heterocyclyl; and
each of L¹ and L² is independently optionally substituted C₂₋₉ heterocyclylene, optionally substituted C₂₋₉ heteroarylene, optionally substituted C₆₋₁₀ arylene, or optionally substituted C₃₋₈ cycloalkylene.

2. The compound of claim 1, or a pharmaceutically acceptable salt thereof, wherein R⁶ is hydrogen, optionally substituted C₆₋₁₀ aryl, optionally substituted C₁₋₉ heteroaryl, optionally substituted C₁₋₆ alkyl, optionally substituted C₂₋₉ heterocyclyl, —OR^{6A}, optionally substituted C₁₋₆ heteroalkyl, or optionally substituted C₃₋₈ cycloalkyl.

3-7. (canceled)

8. The compound of claim 1, or a pharmaceutically acceptable salt thereof, wherein R⁶ is -L¹-R^{6C};
wherein
L¹ is optionally substituted C₂₋₉ heteroarylene;
R^{6C} is optionally substituted C₂₋₉ heterocyclyl, —OR^{6D}, or —N(R^{6E})₂;
R^{6D} is optionally substituted C₁₋₆ alkyl; and
each R^{6E} is independently hydrogen, optionally substituted C₁₋₆ alkyl, or optionally substituted C₂₋₉ heterocyclyl.

9-23. (canceled)

24. The compound of claim 1, or a pharmaceutically acceptable salt thereof, wherein R⁶ is:

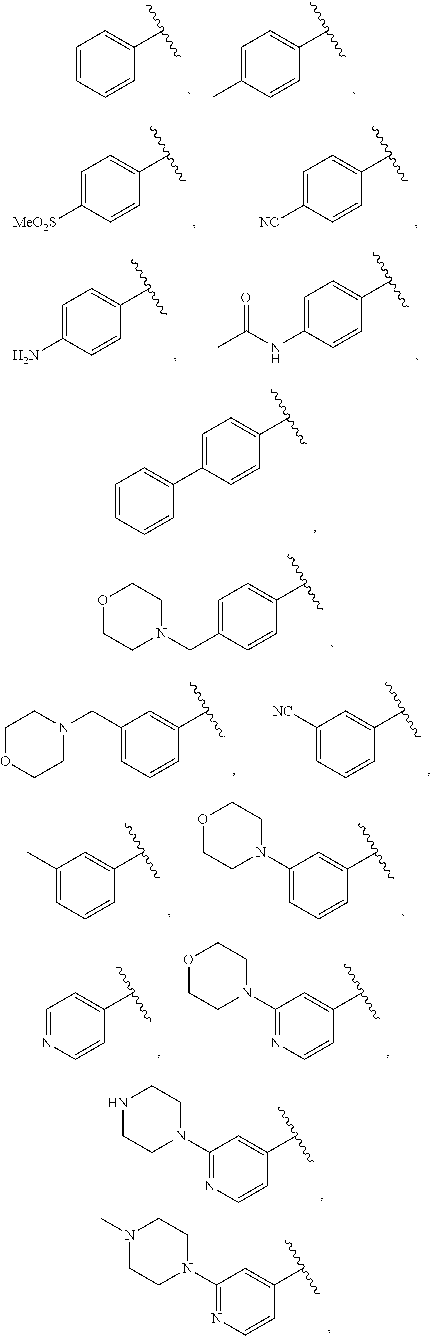

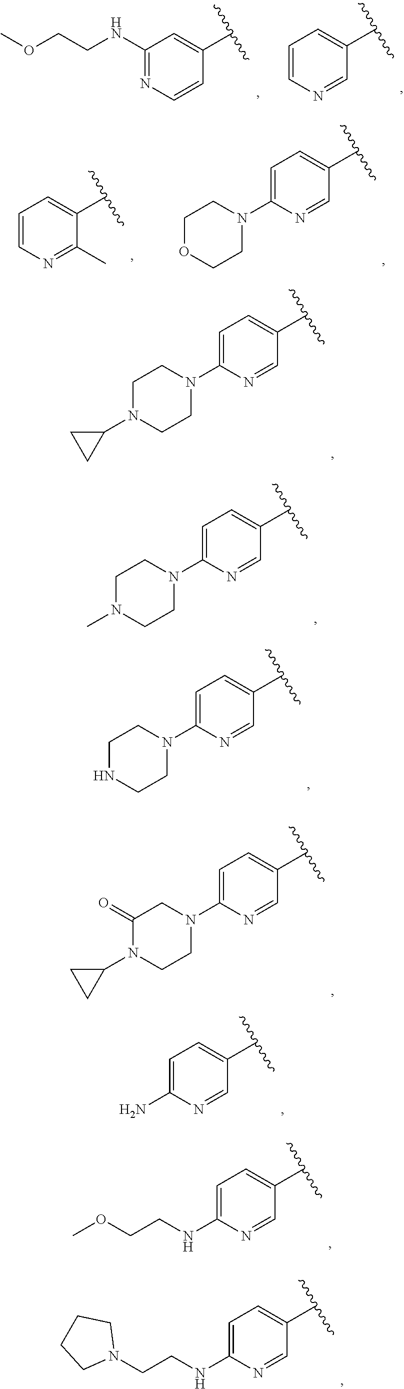

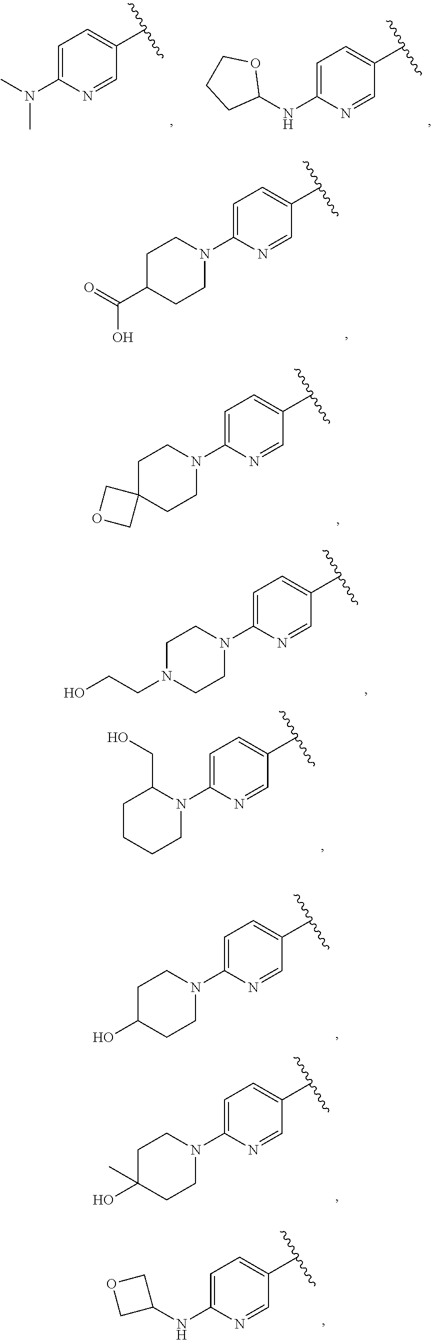

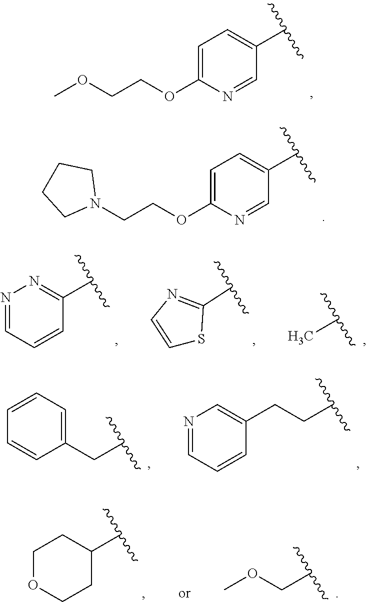

25-31. (canceled)

32. The compound of claim 1, or a pharmaceutically acceptable salt thereof,
wherein the compound is of formula (III-A), (III-B), (II-C), (II-A), or (II-B): 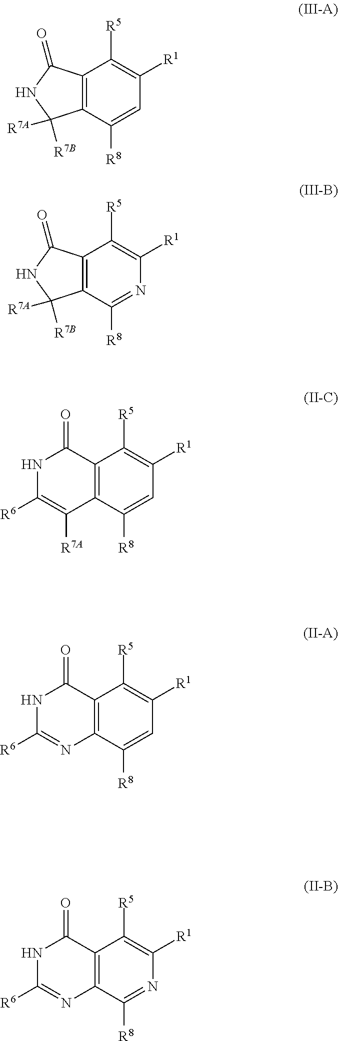
wherein
R^{7B} is hydrogen;
R^{7A} is hydrogen; and
R⁵ is N(R^{5A})₂.

33-40. (canceled)

41. The compound of claim 1, or a pharmaceutically acceptable salt thereof, wherein R¹ is:

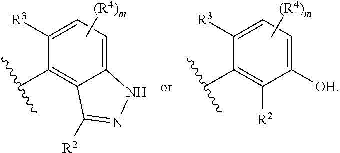

42. The compound of claim 41, or a pharmaceutically acceptable salt thereof, wherein R¹ is enriched for the atropisomer having the structure of: 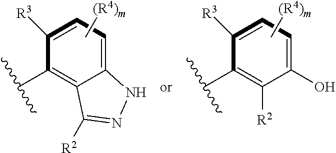
wherein
m is 0, 1, or 2;
R⁴ is fluoro;
R² is optionally substituted C₁₋₆ alkyl or hydrogen; and
R³ is optionally substituted C₁₋₆ alkyl or hydrogen.

43-52. (canceled)

53. The compound of any one of claim 1, or a pharmaceutically acceptable salt thereof, wherein R¹ is:

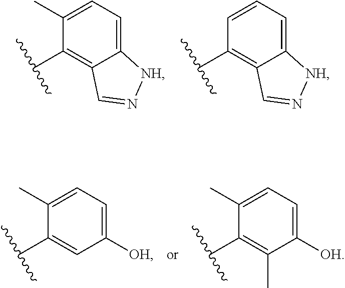

54-56. (canceled)

57. The compound of claim 1, or a pharmaceutically acceptable salt thereof, wherein R⁸ is hydrogen, halogen, optionally substituted C₆₋₁₀ aryl, optionally substituted C₂₋₉ heterocyclyl, optionally substituted C₃₋₈ cycloalkyl, or optionally substituted C₁₋₉ heteroaryl.

58-66. (canceled)

67. The compound of claim 1, or a pharmaceutically acceptable salt thereof, wherein R⁸ is -L²-R^{8B} and L² is optionally substituted C₆₋₁₀ arylene or optionally substituted C₂₋₉ heteroarylene.

68-70. (canceled)

71. The compound of claim 1, or a pharmaceutically acceptable salt thereof, wherein -L²-R^{8B} is: 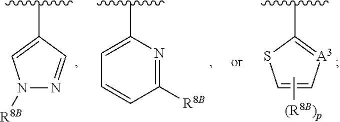
wherein
A³ is N or CH; and
p is 0, 1, or 2.

72-78. (canceled)

79. The compound of claim 71, or a pharmaceutically acceptable salt thereof, wherein R^{8B} is cyano, halogen, optionally substituted acyl, —C(O)N(R^{8E})₂, optionally substituted C₁₋₆ alkyl, —CF₃, or OR^{8C},
wherein R^{8C} is optionally substituted C₁₋₆ alkyl or optionally substituted C₂₋₉ heterocyclyl; and
one R^{8E} is hydrogen, optionally substituted C₁₋₆ heteroalkyl, optionally substituted C₁₋₆ alkyl, or optionally substituted C₃₋₈ cycloalkyl; or
two R^{8E} groups, together with the atom to which both are attached, combine to form an optionally substituted C₂₋₉ heterocyclyl.

80-91. (canceled)

92. The compound of claim 79, or a pharmaceutically acceptable salt thereof, wherein —C(O)N(R^{8E})₂ is:

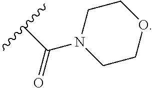 [SMILES: *C(=O)N1CCOCC1]

93-96. (canceled)

97. The compound of claim 1, or a pharmaceutically acceptable salt thereof, wherein R⁸ is:

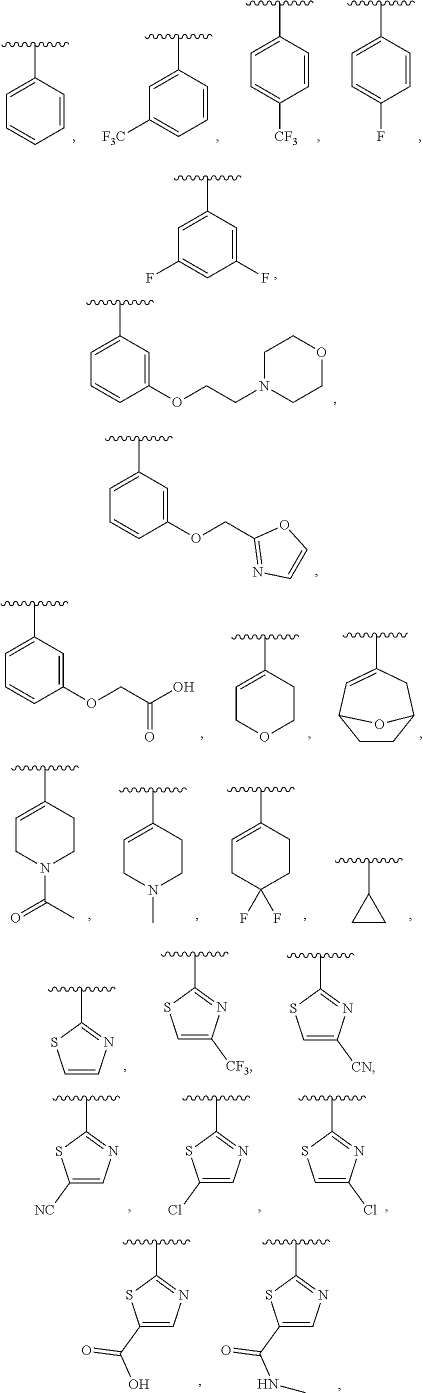

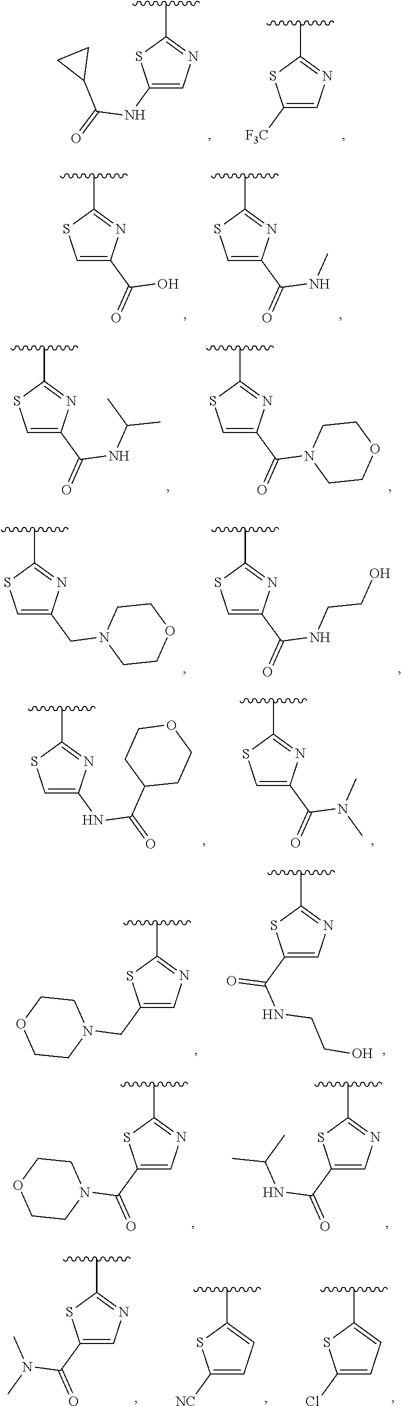

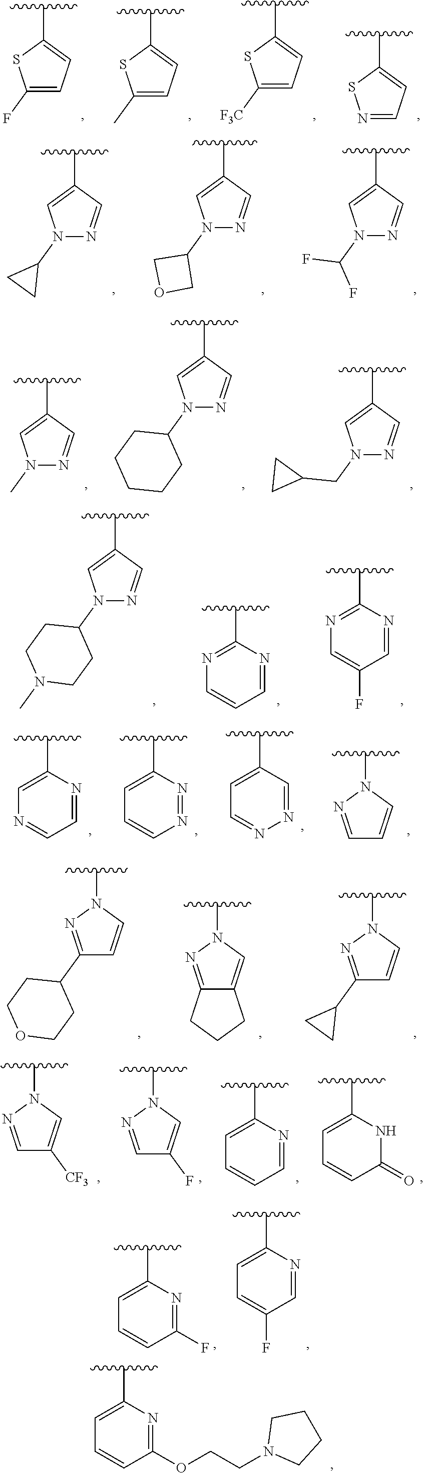

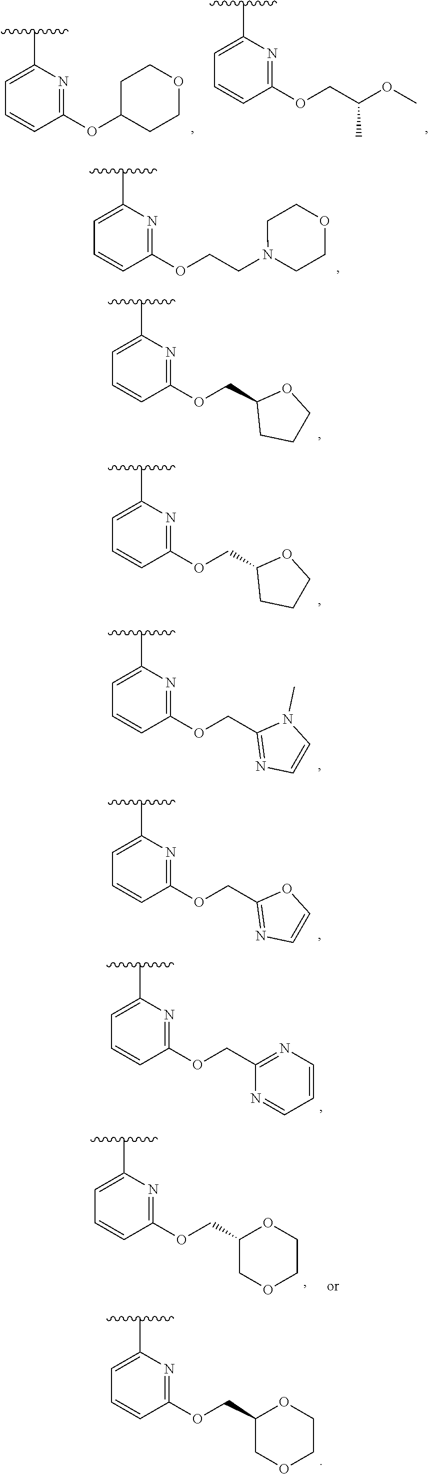

98-107. (canceled)

108. A compound selected from the group consisting of compounds 1 to 165 and pharmaceutically acceptable salts thereof.

109. A pharmaceutical composition comprising the compound of claim 1, or a pharmaceutically acceptable salt thereof, and a pharmaceutically acceptable excipient.

110. (canceled)

111. A method of treating a cancer in a subject, the method comprising administering to the subject in need thereof a therapeutically effective amount of the compound of claim 1, or a pharmaceutically acceptable salt thereof, or a pharmaceutical composition thereof, wherein the cancer either is or has been previously identified as a cancer overexpressing CCNE1 or having an inactivation mutation in the FBWX7 gene.

112. (canceled)

113. A method of inducing cell death in a cancer cell overexpressing CCNE1 or in a FBXW7-mutated cancer cell, the method comprising contacting the cell with an effective amount of the compound of claim 1, or a pharmaceutically acceptable salt thereof.

114. The method of claim 111, wherein the cancer is uterine cancer, ovarian cancer, breast cancer, stomach cancer, esophageal cancer, lung cancer, endometrial cancer, or colorectal cancer.

115-118. (canceled)

119. The method of claim 114, wherein the cell is in a subject.
